SEQUENCE LISTING (122 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 83 aa, synthetic construct
  REGION 1..83: note = Human PTH 1-83
SVSEIQLMHNLGKHLNSMERVEWLRKKLQDVHNFVALGAPLAPRDAGSQRPRKKEDNVLVESHEKSLGEADKADVNVLTKAKS

SEQ ID NO: 3 - protein, 82 aa, synthetic construct
  REGION 1..82: note = human PTH 1-82
SVSEIQLMHNLGKHLNSMERVEWLRKKLQDVHNFVALGAPLAPRDAGSQRPRKKEDNVLVESHEKSLGEADKADVNVLTKAK

SEQ ID NO: 4 - protein, 81 aa, synthetic construct
  REGION 1..81: note = human PTH 1-81
SVSEIQLMHNLGKHLNSMERVEWLRKKLQDVHNFVALGAPLAPRDAGSQRPRKKEDNVLVESHEKSLGEADKADVNVLTKA

SEQ ID NO: 5 - protein, 80 aa, synthetic construct
  REGION 1..80: note = human PTH 1-80
SVSEIQLMHNLGKHLNSMERVEWLRKKLQDVHNFVALGAPLAPRDAGSQRPRKKEDNVLVESHEKSLGEADKADVNVLTK

SEQ ID NO: 6 - protein, 79 aa, synthetic construct
  REGION 1..79: note = human PTH 1-79
SVSEIQLMHNLGKHLNSMERVEWLRKKLQDVHNFVALGAPLAPRDAGSQRPRKKEDNVLVESHEKSLGEADKADVNVLT

SEQ ID NO: 7 - protein, 78 aa, synthetic construct
  REGION 1..78: note = human PTH 1-78
SVSEIQLMHNLGKHLNSMERVEWLRKKLQDVHNFVALGAPLAPRDAGSQRPRKKEDNVLVESHEKSLGEADKADVNVL

SEQ ID NO: 8 - protein, 77 aa, synthetic construct
  REGION 1..77: note = human PTH 1-77
SVSEIQLMHNLGKHLNSMERVEWLRKKLQDVHNFVALGAPLAPRDAGSQRPRKKEDNVLVESHEKSLGEADKADVNV

SEQ ID NO: 9 - protein, 76 aa, synthetic construct
  REGION 1..76: note = human PTH 1-76
SVSEIQLMHNLGKHLNSMERVEWLRKKLQDVHNFVALGAPLAPRDAGSQRPRKKEDNVLVESHEKSLGEADKADVN

SEQ ID NO: 10 - protein, 75 aa, synthetic construct
  REGION 1..75: note = human PTH 1-75
SVSEIQLMHNLGKHLNSMERVEWLRKKLQDVHNFVALGAPLAPRDAGSQRPRKKEDNVLVESHEKSLGEADKADV

SEQ ID NO: 11 - protein, 74 aa, synthetic construct
  REGION 1..74: note = human PTH 1-74
SVSEIQLMHNLGKHLNSMERVEWLRKKLQDVHNFVALGAPLAPRDAGSQRPRKKEDNVLVESHEKSLGEADKAD

SEQ ID NO: 12 - protein, 73 aa, synthetic construct
  REGION 1..73: note = human PTH 1-73
SVSEIQLMHNLGKHLNSMERVEWLRKKLQDVHNFVALGAPLAPRDAGSQRPRKKEDNVLVESHEKSLGEADKA

SEQ ID NO: 13 - protein, 72 aa, synthetic construct
  REGION 1..72: note = human PTH 1-72
SVSEIQLMHNLGKHLNSMERVEWLRKKLQDVHNFVALGAPLAPRDAGSQRPRKKEDNVLVESHEKSLGEADK

SEQ ID NO: 14 - protein, 71 aa, synthetic construct
  REGION 1..71: note = human PTH 1-71
SVSEIQLMHNLGKHLNSMERVEWLRKKLQDVHNFVALGAPLAPRDAGSQRPRKKEDNVLVESHEKSLGEAD

SEQ ID NO: 15 - protein, 70 aa, synthetic construct
  REGION 1..70: note = human PTH 1-70
SVSEIQLMHNLGKHLNSMERVEWLRKKLQDVHNFVALGAPLAPRDAGSQRPRKKEDNVLVESHEKSLGEA

SEQ ID NO: 16 - protein, 69 aa, synthetic construct
  REGION 1..69: note = human PTH 1-69
SVSEIQLMHNLGKHLNSMERVEWLRKKLQDVHNFVALGAPLAPRDAGSQRPRKKEDNVLVESHEKSLGE

SEQ ID NO: 17 - protein, 68 aa, synthetic construct
  REGION 1..68: note = human PTH 1-68
SVSEIQLMHNLGKHLNSMERVEWLRKKLQDVHNFVALGAPLAPRDAGSQRPRKKEDNVLVESHEKSLG

SEQ ID NO: 18 - protein, 67 aa, synthetic construct
  REGION 1..67: note = human PTH 1-67
SVSEIQLMHNLGKHLNSMERVEWLRKKLQDVHNFVALGAPLAPRDAGSQRPRKKEDNVLVESHEKSL

SEQ ID NO: 19 - protein, 66 aa, synthetic construct
  REGION 1..66: note = human PTH 1-66
SVSEIQLMHNLGKHLNSMERVEWLRKKLQDVHNFVALGAPLAPRDAGSQRPRKKEDNVLVESHEKS

SEQ ID NO: 20 - protein, 65 aa, synthetic construct
  REGION 1..65: note = human PTH 1-65
SVSEIQLMHNLGKHLNSMERVEWLRKKLQDVHNFVALGAPLAPRDAGSQRPRKKEDNVLVESHEK

SEQ ID NO: 21 - protein, 64 aa, synthetic construct
  REGION 1..64: note = human PTH 1-64
SVSEIQLMHNLGKHLNSMERVEWLRKKLQDVHNFVALGAPLAPRDAGSQRPRKKEDNVLVESHE

SEQ ID NO: 22 - protein, 63 aa, synthetic construct
  REGION 1..63: note = human PTH 1-63
SVSEIQLMHNLGKHLNSMERVEWLRKKLQDVHNFVALGAPLAPRDAGSQRPRKKEDNVLVESH

SEQ ID NO: 23 - protein, 62 aa, synthetic construct
  REGION 1..62: note = human PTH 1-62
SVSEIQLMHNLGKHLNSMERVEWLRKKLQDVHNFVALGAPLAPRDAGSQRPRKKEDNVLVES

SEQ ID NO: 24 - protein, 61 aa, synthetic construct
  REGION 1..61: note = human PTH 1-61
SVSEIQLMHNLGKHLNSMERVEWLRKKLQDVHNFVALGAPLAPRDAGSQRPRKKEDNVLVE

SEQ ID NO: 25 - protein, 60 aa, synthetic construct
  REGION 1..60: note = human PTH 1-60
SVSEIQLMHNLGKHLNSMERVEWLRKKLQDVHNFVALGAPLAPRDAGSQRPRKKEDNVLV

SEQ ID NO: 26 - protein, 59 aa, synthetic construct
  REGION 1..59: note = human PTH 1-59
SVSEIQLMHNLGKHLNSMERVEWLRKKLQDVHNFVALGAPLAPRDAGSQRPRKKEDNVL

SEQ ID NO: 27 - protein, 58 aa, synthetic construct
  REGION 1..58: note = human PTH 1-58
SVSEIQLMHNLGKHLNSMERVEWLRKKLQDVHNFVALGAPLAPRDAGSQRPRKKEDNV

SEQ ID NO: 28 - protein, 57 aa, synthetic construct
  REGION 1..57: note = human PTH 1-57
SVSEIQLMHNLGKHLNSMERVEWLRKKLQDVHNFVALGAPLAPRDAGSQRPRKKEDN

SEQ ID NO: 29 - protein, 56 aa, synthetic construct
  REGION 1..56: note = human PTH 1-56
SVSEIQLMHNLGKHLNSMERVEWLRKKLQDVHNFVALGAPLAPRDAGSQRPRKKED

SEQ ID NO: 30 - protein, 55 aa, synthetic construct
  REGION 1..55: note = human PTH 1-55
SVSEIQLMHNLGKHLNSMERVEWLRKKLQDVHNFVALGAPLAPRDAGSQRPRKKE

SEQ ID NO: 31 - protein, 54 aa, synthetic construct
  REGION 1..54: note = human PTH 1-54
SVSEIQLMHNLGKHLNSMERVEWLRKKLQDVHNFVALGAPLAPRDAGSQRPRKK

SEQ ID NO: 32 - protein, 53 aa, synthetic construct
  REGION 1..53: note = human PTH 1-53
SVSEIQLMHNLGKHLNSMERVEWLRKKLQDVHNFVALGAPLAPRDAGSQRPRK

SEQ ID NO: 33 - protein, 52 aa, synthetic construct
  REGION 1..52: note = human PTH 1-52
SVSEIQLMHNLGKHLNSMERVEWLRKKLQDVHNFVALGAPLAPRDAGSQRPR

SEQ ID NO: 34 - protein, 51 aa, synthetic construct
  REGION 1..51: note = human PTH 1-51
SVSEIQLMHNLGKHLNSMERVEWLRKKLQDVHNFVALGAPLAPRDAGSQRP

SEQ ID NO: 35 - protein, 50 aa, synthetic construct
  REGION 1..50: note = human PTH 1-50
SVSEIQLMHNLGKHLNSMERVEWLRKKLQDVHNFVALGAPLAPRDAGSQR

SEQ ID NO: 36 - protein, 49 aa, synthetic construct
  REGION 1..49: note = human PTH 1-49
SVSEIQLMHNLGKHLNSMERVEWLRKKLQDVHNFVALGAPLAPRDAGSQ

SEQ ID NO: 37 - protein, 48 aa, synthetic construct
  REGION 1..48: note = human PTH 1-48
SVSEIQLMHNLGKHLNSMERVEWLRKKLQDVHNFVALGAPLAPRDAGS

SEQ ID NO: 38 - protein, 47 aa, synthetic construct
  REGION 1..47: note = human PTH 1-47
SVSEIQLMHNLGKHLNSMERVEWLRKKLQDVHNFVALGAPLAPRDAG

SEQ ID NO: 39 - protein, 46 aa, synthetic construct
  REGION 1..46: note = human PTH 1-46
SVSEIQLMHNLGKHLNSMERVEWLRKKLQDVHNFVALGAPLAPRDA

SEQ ID NO: 40 - protein, 45 aa, synthetic construct
  REGION 1..45: note = human PTH 1-45
SVSEIQLMHNLGKHLNSMERVEWLRKKLQDVHNFVALGAPLAPRD

SEQ ID NO: 41 - protein, 44 aa, synthetic construct
  REGION 1..44: note = human PTH 1-44
SVSEIQLMHNLGKHLNSMERVEWLRKKLQDVHNFVALGAPLAPR

SEQ ID NO: 42 - protein, 43 aa, synthetic construct
  REGION 1..43: note = human PTH 1-43
SVSEIQLMHNLGKHLNSMERVEWLRKKLQDVHNFVALGAPLAP

SEQ ID NO: 43 - protein, 42 aa, synthetic construct
  REGION 1..42: note = human PTH 1-42
SVSEIQLMHNLGKHLNSMERVEWLRKKLQDVHNFVALGAPLA

SEQ ID NO: 44 - protein, 41 aa, synthetic construct
  REGION 1..41: note = human PTH-41
SVSEIQLMHNLGKHLNSMERVEWLRKKLQDVHNFVALGAPL

SEQ ID NO: 45 - protein, 40 aa, synthetic construct
  REGION 1..40: note = human PTH 1-40
SVSEIQLMHNLGKHLNSMERVEWLRKKLQDVHNFVALGAP

SEQ ID NO: 46 - protein, 39 aa, synthetic construct
  REGION 1..39: note = human PTH 1-39
SVSEIQLMHNLGKHLNSMERVEWLRKKLQDVHNFVALGA

SEQ ID NO: 47 - protein, 38 aa, synthetic construct
  REGION 1..38: note = human PTH 1-38
SVSEIQLMHNLGKHLNSMERVEWLRKKLQDVHNFVALG

SEQ ID NO: 48 - protein, 37 aa, synthetic construct
  REGION 1..37: note = human PTH 1-37
SVSEIQLMHNLGKHLNSMERVEWLRKKLQDVHNFVAL

SEQ ID NO: 49 - protein, 36 aa, synthetic construct
  REGION 1..36: note = human PTH 1-36
SVSEIQLMHNLGKHLNSMERVEWLRKKLQDVHNFVA

SEQ ID NO: 50 - protein, 35 aa, synthetic construct
  REGION 1..35: note = human PTH 1-35
SVSEIQLMHNLGKHLNSMERVEWLRKKLQDVHNFV

SEQ ID NO: 51 - protein, 34 aa, synthetic construct
  REGION 1..34: note = human PTH 1-34
SVSEIQLMHNLGKHLNSMERVEWLRKKLQDVHNF

SEQ ID NO: 52 - protein, 33 aa, synthetic construct
  REGION 1..33: note = human PTH 1-33
SVSEIQLMHNLGKHLNSMERVEWLRKKLQDVHN

SEQ ID NO: 53 - protein, 32 aa, synthetic construct
  REGION 1..32: note = human PTH 1-32
SVSEIQLMHNLGKHLNSMERVEWLRKKLQDVH

SEQ ID NO: 54 - protein, 31 aa, synthetic construct
  REGION 1..31: note = human PTH 1-31
SVSEIQLMHNLGKHLNSMERVEWLRKKLQDV

SEQ ID NO: 55 - protein, 30 aa, synthetic construct
  REGION 1..30: note = human PTH 1-30
SVSEIQLMHNLGKHLNSMERVEWLRKKLQD

SEQ ID NO: 56 - protein, 29 aa, synthetic construct
  REGION 1..29: note = human PTH 1-29
SVSEIQLMHNLGKHLNSMERVEWLRKKLQ

SEQ ID NO: 57 - protein, 28 aa, synthetic construct
  REGION 1..28: note = human PTH 1-28
SVSEIQLMHNLGKHLNSMERVEWLRKKL

SEQ ID NO: 58 - protein, 27 aa, synthetic construct
  REGION 1..27: note = human PTH 1-27
SVSEIQLMHNLGKHLNSMERVEWLRKK

SEQ ID NO: 59 - protein, 26 aa, synthetic construct
  REGION 1..26: note = human PTH 1-26
SVSEIQLMHNLGKHLNSMERVEWLRK

SEQ ID NO: 60 - protein, 25 aa, synthetic construct
  REGION 1..25: note = human PTH 1-25
SVSEIQLMHNLGKHLNSMERVEWLR

SEQ ID NO: 61 - protein, 84 aa, synthetic construct; identical to SEQ ID NO: 1
  REGION 1..84: note = amidated human PTH 1-84
  MOD_RES 84: note = AMIDATION

SEQ ID NO: 62 - protein, 83 aa, synthetic construct; identical to SEQ ID NO: 2
  REGION 1..83: note = amidated human PTH 1-83
  MOD_RES 83: note = AMIDATION

SEQ ID NO: 63 - protein, 82 aa, synthetic construct; identical to SEQ ID NO: 3
  REGION 1..82: note = amidated human PTH 1-82
  MOD_RES 82: note = AMIDATION

SEQ ID NO: 64 - protein, 81 aa, synthetic construct; identical to SEQ ID NO: 4
  REGION 1..81: note = amidated human PTH 1-81
  MOD_RES 81: note = AMIDATION

SEQ ID NO: 65 - protein, 80 aa, synthetic construct; identical to SEQ ID NO: 5
  REGION 1..80: note = amidated human PTH 1-80
  MOD_RES 80: note = AMIDATION

SEQ ID NO: 66 - protein, 79 aa, synthetic construct; identical to SEQ ID NO: 6
  REGION 1..79: note = amidated human PTH 1-79
  MOD_RES 79: note = AMIDATION

SEQ ID NO: 67 - protein, 78 aa, synthetic construct; identical to SEQ ID NO: 7
  REGION 1..78: note = amidated human PTH 1-78
  MOD_RES 78: note = AMIDATION

SEQ ID NO: 68 - protein, 77 aa, synthetic construct; identical to SEQ ID NO: 8
  REGION 1..77: note = amidated human PTH 1-77
  MOD_RES 77: note = AMIDATION

SEQ ID NO: 69 - protein, 76 aa, synthetic construct; identical to SEQ ID NO: 9
  REGION 1..76: note = amidated human PTH 1-76
  MOD_RES 76: note = AMIDATION

SEQ ID NO: 70 - protein, 75 aa, synthetic construct; identical to SEQ ID NO: 10
  REGION 1..75: note = amidated human PTH 1-75
  MOD_RES 75: note = AMIDATION

SEQ ID NO: 71 - protein, 74 aa, synthetic construct; identical to SEQ ID NO: 11
  REGION 1..74: note = amidated human PTH 1-74
  MOD_RES 74: note = AMIDATION

SEQ ID NO: 72 - protein, 73 aa, synthetic construct; identical to SEQ ID NO: 12
  REGION 1..73: note = amidated human PTH 1-73
  MOD_RES 73: note = AMIDATION

SEQ ID NO: 73 - protein, 72 aa, synthetic construct; identical to SEQ ID NO: 13
  REGION 1..72: note = amidated human PTH 1-72
  MOD_RES 72: note = AMIDATION

SEQ ID NO: 74 - protein, 71 aa, synthetic construct; identical to SEQ ID NO: 14
  REGION 1..71: note = amidated human PTH 1-71
  MOD_RES 71: note = AMIDATION

SEQ ID NO: 75 - protein, 70 aa, synthetic construct; identical to SEQ ID NO: 15
  REGION 1..70: note = amidated human PTH 1-70
  MOD_RES 70: note = AMIDATION

SEQ ID NO: 76 - protein, 69 aa, synthetic construct; identical to SEQ ID NO: 16
  REGION 1..69: note = amidated human PTH 1-69
  MOD_RES 69: note = AMIDATION

SEQ ID NO: 77 - protein, 68 aa, synthetic construct; identical to SEQ ID NO: 17
  REGION 1..68: note = amidated human PTH 1-68
  MOD_RES 68: note = AMIDATION

SEQ ID NO: 78 - protein, 67 aa, synthetic construct; identical to SEQ ID NO: 18
  REGION 1..67: note = amidated human PTH 1-67
  MOD_RES 67: note = AMIDATION

SEQ ID NO: 79 - protein, 66 aa, synthetic construct; identical to SEQ ID NO: 19
  REGION 1..66: note = amidated human PTH 1-66
  MOD_RES 66: note = AMIDATION

SEQ ID NO: 80 - protein, 65 aa, synthetic construct; identical to SEQ ID NO: 20
  REGION 1..65: note = amidated human PTH 1-65
  MOD_RES 65: note = AMIDATION

SEQ ID NO: 81 - protein, 64 aa, synthetic construct; identical to SEQ ID NO: 21
  REGION 1..64: note = amidated human PTH 1-64
  MOD_RES 64: note = AMIDATION

SEQ ID NO: 82 - protein, 63 aa, synthetic construct; identical to SEQ ID NO: 22
  REGION 1..63: note = amidated human PTH 1-63
  MOD_RES 63: note = AMIDATION

SEQ ID NO: 83 - protein, 62 aa, synthetic construct; identical to SEQ ID NO: 23
  REGION 1..62: note = amidated human PTH 1-62
  MOD_RES 62: note = AMIDATION

SEQ ID NO: 84 - protein, 61 aa, synthetic construct; identical to SEQ ID NO: 24
  REGION 1..61: note = amidated human PTH 1-61
  MOD_RES 61: note = AMIDATION

SEQ ID NO: 85 - protein, 60 aa, synthetic construct; identical to SEQ ID NO: 25
  REGION 1..60: note = amidated human PTH 1-60
  MOD_RES 60: note = ACETYLATION

SEQ ID NO: 86 - protein, 59 aa, synthetic construct; identical to SEQ ID NO: 26
  REGION 1..59: note = amidated human PTH 1-59
  MOD_RES 59: note = AMIDATION

SEQ ID NO: 87 - protein, 58 aa, synthetic construct; identical to SEQ ID NO: 27
  REGION 1..58: note = amidated human PTH 1-58
  MOD_RES 58: note = AMIDATION

SEQ ID NO: 88 - protein, 57 aa, synthetic construct; identical to SEQ ID NO: 28
  REGION 1..57: note = amidated human PTH 1-57
  MOD_RES 57: note = AMIDATION

SEQ ID NO: 89 - protein, 56 aa, synthetic construct; identical to SEQ ID NO: 29
  REGION 1..56: note = amidated human PTH 1-56
  MOD_RES 56: note = AMIDATION

SEQ ID NO: 90 - protein, 55 aa, synthetic construct; identical to SEQ ID NO: 30
  REGION 1..55: note = amidated human PTH 1-55
  MOD_RES 55: note = AMIDATION

SEQ ID NO: 91 - protein, 54 aa, synthetic construct; identical to SEQ ID NO: 31
  REGION 1..54: note = amidated human PTH 1-54
  MOD_RES 54: note = AMIDATION

SEQ ID NO: 92 - protein, 53 aa, synthetic construct; identical to SEQ ID NO: 32
  REGION 1..53: note = amidated human PTH 1-53
  MOD_RES 53: note = AMIDATION

SEQ ID NO: 93 - protein, 52 aa, synthetic construct; identical to SEQ ID NO: 33
  REGION 1..52: note = amidated human PTH 1-52
  MOD_RES 52: note = AMIDATION

SEQ ID NO: 94 - protein, 51 aa, synthetic construct; identical to SEQ ID NO: 34
  REGION 1..51: note = amidated human PTH 1-51
  MOD_RES 51: note = AMIDATION

SEQ ID NO: 95 - protein, 50 aa, synthetic construct; identical to SEQ ID NO: 35
  REGION 1..50: note = amidated human PTH 1-50
  MOD_RES 50: note = AMIDATION

SEQ ID NO: 96 - protein, 49 aa, synthetic construct; identical to SEQ ID NO: 36
  REGION 1..49: note = amidated human PTH 1-49
  MOD_RES 49: note = AMIDATION

SEQ ID NO: 97 - protein, 48 aa, synthetic construct; identical to SEQ ID NO: 37
  REGION 1..48: note = amidated human PTH 1-48
  MOD_RES 48: note = AMIDATION

SEQ ID NO: 98 - protein, 47 aa, synthetic construct; identical to SEQ ID NO: 38
  REGION 1..47: note = amidated human PTH 1-47
  MOD_RES 47: note = AMIDATION

SEQ ID NO: 99 - protein, 46 aa, synthetic construct; identical to SEQ ID NO: 39
  REGION 1..46: note = amidated human PTH 1-46
  MOD_RES 46: note = AMIDATION

SEQ ID NO: 100 - protein, 45 aa, synthetic construct; identical to SEQ ID NO: 40
  REGION 1..45: note = amidated human PTH 1-45
  MOD_RES 45: note = AMIDATION

SEQ ID NO: 101 - protein, 44 aa, synthetic construct; identical to SEQ ID NO: 41
  REGION 1..44: note = amidated human PTH 1-44
  MOD_RES 44: note = AMIDATION

SEQ ID NO: 102 - protein, 43 aa, synthetic construct; identical to SEQ ID NO: 42
  REGION 1..43: note = amidated human PTH 1-43
  MOD_RES 43: note = AMIDATION

SEQ ID NO: 103 - protein, 42 aa, synthetic construct; identical to SEQ ID NO: 43
  REGION 1..42: note = amidated human PTH 1-42
  MOD_RES 42: note = AMIDATION

SEQ ID NO: 104 - protein, 41 aa, synthetic construct; identical to SEQ ID NO: 44
  REGION 1..41: note = amidated human PTH 1-41
  MOD_RES 41: note = AMIDATION

SEQ ID NO: 105 - protein, 40 aa, synthetic construct; identical to SEQ ID NO: 45
  REGION 1..40: note = amidated human PTH 1-40
  MOD_RES 40: note = AMIDATION

SEQ ID NO: 106 - protein, 39 aa, synthetic construct; identical to SEQ ID NO: 46
  REGION 1..39: note = amidated human PTH 1-39
  MOD_RES 39: note = AMIDATION

SEQ ID NO: 107 - protein, 38 aa, synthetic construct; identical to SEQ ID NO: 47
  REGION 1..38: note = amidated human PTH 1-38
  MOD_RES 38: note = AMIDATION

SEQ ID NO: 108 - protein, 37 aa, synthetic construct; identical to SEQ ID NO: 48
  REGION 1..37: note = amidated human PTH 1-37
  MOD_RES 37: note = AMIDATION

SEQ ID NO: 109 - protein, 36 aa, synthetic construct; identical to SEQ ID NO: 49
  REGION 1..36: note = amidated human PTH 1-36
  MOD_RES 36: note = AMIDATION

SEQ ID NO: 110 - protein, 35 aa, synthetic construct; identical to SEQ ID NO: 50
  REGION 1..35: note = amidated human PTH 1-35
  MOD_RES 35: note = AMIDATION

SEQ ID NO: 111 - protein, 34 aa, synthetic construct; identical to SEQ ID NO: 51
  REGION 1..34: note = amidated human PTH 1-34
  MOD_RES 34: note = AMIDATION

SEQ ID NO: 112 - protein, 33 aa, synthetic construct; identical to SEQ ID NO: 52
  REGION 1..33: note = amidated human PTH 1-33
  MOD_RES 33: note = AMIDATION

SEQ ID NO: 113 - protein, 32 aa, synthetic construct; identical to SEQ ID NO: 53
  REGION 1..32: note = amidated human PTH 1-32
  MOD_RES 32: note = AMIDATION

SEQ ID NO: 114 - protein, 31 aa, synthetic construct; identical to SEQ ID NO: 54
  REGION 1..31: note = amidated human PTH 1-31
  MOD_RES 31: note = AMIDATION

SEQ ID NO: 115 - protein, 30 aa, synthetic construct; identical to SEQ ID NO: 55
  REGION 1..30: note = amidated human PTH 1-30
  MOD_RES 30: note = AMIDATION

SEQ ID NO: 116 - protein, 29 aa, synthetic construct
  REGION 1..29: note = amidated human PTH 1-29
  MOD_RES 29: note = AMIDATION
SVSEIQLMHNLGKHLNSMERVEWLRKKLQ

SEQ ID NO: 117 - protein, 28 aa, synthetic construct
  REGION 1..28: note = amidated human PTH 1-28
  MOD_RES 28: note = AMIDATION
SVSEIQLMHNLGKHLNSMERVEWLRKKL

SEQ ID NO: 118 - protein, 27 aa, synthetic construct
  REGION 1..27: note = amidated human PTH 1-27
  MOD_RES 27: note = AMIDATION
SVSEIQLMHNLGKHLNSMERVEWLRKK

SEQ ID NO: 119 - protein, 26 aa, synthetic construct
  REGION 1..26: note = amidated human PTH 1-26
  MOD_RES 26: note = AMIDATION
SVSEIQLMHNLGKHLNSMERVEWLRK

SEQ ID NO: 120 - protein, 25 aa, synthetic construct
  REGION 1..25: note = amidated human PTH 1-25
  MOD_RES 25: note = AMIDATION
SVSEIQLMHNLGKHLNSMERVEWLR

SEQ ID NO: 121 - protein, 141 aa, Homo sapiens
AVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 122 - protein, 36 aa, synthetic construct
AVAEIQLMHQRAKWIQDARRRAFLHKLIAEIHTAEI

(19) United States
(12) Patent Application Publication
(10) Pub. No.: US 2024/0382567 A1
(43) Pub. Date: Nov. 21, 2024

(54) Long-acting PTH compound treatments

(71) Applicant: Ascendis Pharma Bone Diseases A/S, Hellerup (DK)

(72) Inventor: Kennett Sprogøe, Hellerup (DK)

(21) Appl. No.: 18/693,510

(22) PCT Filed: Sep. 21, 2022
(86) PCT No.: PCT/EP2022/076188

§ 371 Date: Mar. 19, 2024

(30) Foreign Application Priority Data
Sep. 22, 2021 (EP) 21198228.5
Aug. 31, 2022 (EP) 22193085.2

(51) Int. Cl. A61K 38/29; A61K 31/593; A61K 33/06; A61K 47/60; A61P 19/08

(52) U.S. Cl. CPC A61K 38/29; A61K 31/593; A61K 33/06; A61K 47/60; A61P 19/08

(57) ABSTRACT

The present invention relates to a long-acting PTH compound for use in the reduction of bone mineral density (BMD) in a patient having an increased BMD and to a particular dosage regimen of such long-acting PTH compound and other related aspects.

The present invention relates to a long-acting PTH compound for use in the reduction of bone mineral density (BMD) in a patient having an increased BMD and to a particular dosage regimen of such long-acting PTH compound.

A finding of high bone mineral density (BMD) on routine dual-energy x-ray absorptiometry (DXA) scanning is not infrequent and most commonly reflects degenerative disease. BMD measurement plays an important role in the assessment of osteoporosis and fracture risk. In clinical practice, BMD is most commonly measured using DXA. BMD is then compared against an age-, ethnicity- and gender-specific reference population to compute T- and Z-scores, which are the number of standard deviations a measured BMD differs from the mean BMD of a young adult population (T-score) or age-matched population (Z-score), respectively. In addition to being a valuable diagnostic tool for identifying the most common form of bone disease, osteoporosis, DXA can also be used for identifying individuals with higher-than-average BMD, which is a BMD Z-score of >0.

Daily injections with PTH peptides are widely used for treating diseases of low BMD, especially osteoporosis. When administered as intermittent injections, PTH potently stimulates cortical and trabecular bone growth in humans. In addition to being approved for osteoporosis, PTH is also useful for treating hypoparathyroidism. Indeed, daily injection of PTH(1-84) is approved as an adjunct to calcium and vitamin D to control hypocalcemia in patients with hypoparathyroidism. While this therapy is effective at controlling serum calcium, changes of BMD is observed. Patients with hypoparathyroidism frequently have high BMD, due to reduced bone turnover as a result of insufficient PTH levels, and severity of elevated BMD tends to correlate with duration of disease. Following therapy with PTH(1-84) the already high BMD is further increased at many skeletal sites. For example, lumbar spine BMD increased by 3.2% at 24 months (P=0.01). At year 6, there was a further significant increase compared with year 2 (P=0.003). By year 6, the mean gain from baseline was 3.8% (P=0.004). Femoral neck BMD increased 2.6% (P=0.04) at year 3, but was not different from baseline at year 6. Total hip BMD increased at 3 years (2.10% from baseline, P=0.04) and remained stable through year 6 (2.4% from baseline, P=0.02 from baseline). In contrast, cortical bone loss was observed, and BMD decreased at the distal one third radius at year 3 (P=0.04 vs baseline), subsequently decreasing further at year 6 to a T-score of −0.28, with a mean loss of 4.4% (P=0.0001 vs baseline) at study conclusion. (J Clin Endocrinol Metab 101: 2742-2750, 2016).

Therefore, there is a need for an effective treatment of diseases of the bone associated with changes in BMD that is capable of normalizing BMD.

In a first aspect the present invention relates to a long-acting PTH compound for use in the reduction of bone mineral density (BMD) in a patient having an increased BMD.

It was surprisingly found that daily injections of a long-acting PTH compound lead to an initial increase in bone turnover markers, that normalized with prolonged therapy. The change in bone markers were associated with an initial decrease in BMD at trabecular sites at 26 weeks, and thereafter stabilized and remained in the normal range at week 58. Changes in bone turnover markers were observed in response to therapy. By week 26, mean values for both the anabolic marker P1NP and resorptive marker CTx increased within the normal range or to just above the normal range, respectively. With prolonged therapy, both P1NP and CTx levels fell towards the mid-normal range, reflecting a normalization of bone turnover. This was accompanied by a stabilization of BMD Z-scores. Bone formation markers P1NP and bone resorption marker CTx increased from the low end of normal at baseline, peaked in the initial weeks of treatment, and trended progressively downwards towards age- and sex-matched norms through week 110.

As BMD normalizes and less calcium is being released from the skeleton, for diseases where the administered long acting PTH compound also sustain normal serum calcium levels, doses may need to be titrated upwards to compensate for less released calcium from the bone.

Treatment with daily injections of a long-acting PTH compound demonstrated an initial increase of bone remodeling and a trend towards mobilization of calcium from the overly dense skeleton in adults with hypoparathyroidism. Mean BMD Z-scores remained above zero for all regions of interest (lumbar spine L1-L4, femoral neck, total hip and distal 1/3 radius). At axial sites, participants with longer duration of hypoparathyroidism had higher baseline Z-scores and larger numeric decreases in Z-scores though week 110.

Patients having hypoparathyroidism have abnormally elevated BMD in trabecular bone and the longer they have the disease, the more severe the increase in BMD becomes, whereas cortical bone does not appear to be affected. Administration of a long-acting PTH compound corrected BMD only at those sites where BMD was abnormal, i.e. in trabecular bone, and BMD stabilizes once BMD approaches age- and sex-appropriate norms.

Within the present invention the terms are used having the meaning as follows.

As used herein the term “increased BMD” or “increased mineral bone density” refers to a Z-score above 0, such as to a Z-score that is at least 0.8, at least 1, at least 1.2 or at least 1.5. The Z-score is the number of standard deviations above or below the mean for gender, ethnicity and age-matched healthy population and is calculated from the following equation:

[(measured BMD−age-matched population mean BMD)/age-matched population SD], wherein BMD is measured by dual-energy x-ray absorptiometry (abbreviated as “DXA” or “DEXA”).

As used herein the terms “within normal level” and “within the normal range” with regard to serum calcium (sCa) levels refer to the calcium level ordinarily found in a subject of a given species, sex and age, provided as the range given by the lower limit of normal and the upper limit of normal. In humans, the normal level in certain embodiments corresponds to a serum calcium level of above 8.5 mg/dL (albumin-adjusted). In humans the upper limit of normal is 10.5 mg/dL

As used herein the term “serum calcium above 8.5 mg/dL” and “the upper limit of normal is 10.5 mg/dL” refers to albumin-adjusted calcium concentrations. As used herein the term “albumin-adjusted” with regard to calcium levels means that the measured serum calcium level is corrected for calcium bound to albumin according to the following formula:

albumin-adjusted serum calcium (mg/dL)=measured total Ca (mg/dL)+0.8(4.0−serum albumin [g/dL]).

As used herein the term “PTH” refers to all PTH polypeptides, preferably from mammalian species, more preferably from human and mammalian species, more preferably from human and murine species, as well as their variants, analogs, orthologs, homologs, and derivatives and fragments thereof, that are characterized by raising serum calcium and renal phosphorus excretion and lowering serum phosphorus and renal calcium excretion. The term “PTH” also refers to all PTHrP polypeptides, such as the polypeptide of SEQ ID NO:121, that bind to and activate the common PTH/PTHrP1 receptor. Preferably, the term “PTH” refers to the PTH polypeptide of SEQ ID NO:51 as well as its variants, homologs and derivatives exhibiting essentially the same biological activity, i.e., raising serum calcium and renal phosphorus excretion, and lowering serum phosphorus and renal calcium excretion.

In certain embodiments the term “PTH” refers to the following polypeptide sequences:

(PTH 1-84)
SEQ ID NO: 1
SVSEIQLMHNLGKHLNSMERVEWLRKKLQDVHNFVALGAPLAPRDAGSQRPRKKE
DNVLVESHEKSLGEADKADVNVLTKAKSQ
(PTH 1-83)
SEQ ID NO: 2
SVSEIQLMHNLGKHLNSMERVEWLRKKLQDVHNFVALGAPLAPRDAGSQRPRKKE
DNVLVESHEKSLGEADKADVNVLTKAKS
(PTH 1-82)
SEQ ID NO: 3
SVSEIQLMHNLGKHLNSMERVEWLRKKLQDVHNFVALGAPLAPRDAGSQRPRKKE
DNVLVESHEKSLGEADKADVNVLTKAK
(PTH 1-81)
SEQ ID NO: 4
SVSEIQLMHNLGKHLNSMERVEWLRKKLQDVHNFVALGAPLAPRDAGSQRPRKKE
DNVLVESHEKSLGEADKADVNVLTKA
(PTH 1-80)
SEQ ID NO: 5
SVSEIQLMHNLGKHLNSMERVEWLRKKLQDVHNFVALGAPLAPRDAGSQRPRKKE
DNVLVESHEKSLGEADKADVNVLTK
(PTH 1-79)
SEQ ID NO: 6
SVSEIQLMHNLGKHLNSMERVEWLRKKLQDVHNFVALGAPLAPRDAGSQRPRKKE
DNVLVESHEKSLGEADKADVNVLT
(PTH 1-78)
SEQ ID NO: 7
SVSEIQLMHNLGKHLNSMERVEWLRKKLQDVHNFVALGAPLAPRDAGSQRPRKKE
DNVLVESHEKSLGEADKADVNVL
(PTH 1-77)
SEQ ID NO: 8
SVSEIQLMHNLGKHLNSMERVEWLRKKLQDVHNFVALGAPLAPRDAGSQRPRKKE
DNVLVESHEKSLGEADKADVNV
(PTH 1-76)
SEQ ID NO: 9
SVSEIQLMHNLGKHLNSMERVEWLRKKLQDVHNFVALGAPLAPRDAGSQRPRKKE
DNVLVESHEKSLGEADKADVN
(PTH 1-75)
SEQ ID NO: 10
SVSEIQLMHNLGKHLNSMERVEWLRKKLQDVHNFVALGAPLAPRDAGSQRPRKKE
DNVLVESHEKSLGEADKADV
(PTH 1-74)
SEQ ID NO: 11
SVSEIQLMHNLGKHLNSMERVEWLRKKLQDVHNFVALGAPLAPRDAGSQRPRKKE
DNVLVESHEKSLGEADKAD
(PTH 1-73)
SEQ ID NO: 12
SVSEIQLMHNLGKHLNSMERVEWLRKKLQDVHNFVALGAPLAPRDAGSQRPRKKE
DNVLVESHEKSLGEADKA
(PTH 1-72)
SEQ ID NO: 13
SVSEIQLMHNLGKHLNSMERVEWLRKKLQDVHNFVALGAPLAPRDAGSQRPRKKE
DNVLVESHEKSLGEADK
(PTH 1-71)
SEQ ID NO: 14
SVSEIQLMHNLGKHLNSMERVEWLRKKLQDVHNFVALGAPLAPRDAGSQRPRKKE
DNVLVESHEKSLGEAD
(PTH 1-70)
SEQ ID NO: 15
SVSEIQLMHNLGKHLNSMERVEWLRKKLQDVHNFVALGAPLAPRDAGSQRPRKKE
DNVLVESHEKSLGEA
(PTH 1-69)
SEQ ID NO: 16
SVSEIQLMHNLGKHLNSMERVEWLRKKLQDVHNFVALGAPLAPRDAGSQRPRKKE
DNVLVESHEKSLGE
(PTH 1-68)
SEQ ID NO: 17
SVSEIQLMHNLGKHLNSMERVEWLRKKLQDVHNFVALGAPLAPRDAGSQRPRKKE
DNVLVESHEKSLG
(PTH 1-67)
SEQ ID NO: 18
SVSEIQLMHNLGKHLNSMERVEWLRKKLQDVHNFVALGAPLAPRDAGSQRPRKKE
DNVLVESHEKSL
(PTH 1-66)
SEQ ID NO: 19
SVSEIQLMHNLGKHLNSMERVEWLRKKLQDVHNFVALGAPLAPRDAGSQRPRKKE
DNVLVESHEKS
(PTH 1-65)
SEQ ID NO: 20
SVSEIQLMHNLGKHLNSMERVEWLRKKLQDVHNFVALGAPLAPRDAGSQRPRKKE
DNVLVESHEK
(PTH 1-64)
SEQ ID NO: 21
SVSEIQLMHNLGKHLNSMERVEWLRKKLQDVHNFVALGAPLAPRDAGSQRPRKKE
DNVLVESHE
(PTH 1-63)
SEQ ID NO: 22
SVSEIQLMHNLGKHLNSMERVEWLRKKLQDVHNFVALGAPLAPRDAGSQRPRKKE
DNVLVESH
(PTH 1-62)
SEQ ID NO: 23
SVSEIQLMHNLGKHLNSMERVEWLRKKLQDVHNFVALGAPLAPRDAGSQRPRKKE
DNVLVES
(PTH 1-61)
SEQ ID NO: 24
SVSEIQLMHNLGKHLNSMERVEWLRKKLQDVHNFVALGAPLAPRDAGSQRPRKKE
DNVLVE
(PTH 1-60)
SEQ ID NO: 25
SVSEIQLMHNLGKHLNSMERVEWLRKKLQDVHNFVALGAPLAPRDAGSQRPRKKE
DNVLV
(PTH 1-59)
SEQ ID NO: 26
SVSEIQLMHNLGKHLNSMERVEWLRKKLQDVHNFVALGAPLAPRDAGSQRPRKKE
DNVL
(PTH 1-58)
SEQ ID NO: 27
SVSEIQLMHNLGKHLNSMERVEWLRKKLQDVHNFVALGAPLAPRDAGSQRPRKKE
DNV
(PTH 1-57)
SEQ ID NO: 28
SVSEIQLMHNLGKHLNSMERVEWLRKKLQDVHNFVALGAPLAPRDAGSQRPRKKE
DN
(PTH 1-56)
SEQ ID NO: 29
SVSEIQLMHNLGKHLNSMERVEWLRKKLQDVHNFVALGAPLAPRDAGSQRPRKKE
D
(PTH 1-55)
SEQ ID NO: 30
SVSEIQLMHNLGKHLNSMERVEWLRKKLQDVHNFVALGAPLAPRDAGSQRPRKKE
(PTH 1-54)
SEQ ID NO: 31
SVSEIQLMHNLGKHLNSMERVEWLRKKLQDVHNFVALGAPLAPRDAGSQRPRKK
SEQ ID NO: 32
(PTH 1-53)
SVSEIQLMHNLGKHLNSMERVEWLRKKLQDVHNFVALGAPLAPRDAGSQRPRK
(PTH 1-52)
SEQ ID NO: 33
SVSEIQLMHNLGKHLNSMERVEWLRKKLQDVHNFVALGAPLAPRDAGSQRPR
(PTH 1-51)
SEQ ID NO: 34
SVSEIQLMHNLGKHLNSMERVEWLRKKLQDVHNFVALGAPLAPRDAGSQRP
(PTH 1-50)
SEQ ID NO: 35
SVSEIQLMHNLGKHLNSMERVEWLRKKLQDVHNFVALGAPLAPRDAGSQR
(PTH 1-49)
SEQ ID NO: 36
SVSEIQLMHNLGKHLNSMERVEWLRKKLQDVHNFVALGAPLAPRDAGSQ
(PTH 1-48)
SEQ ID NO: 37
SVSEIQLMHNLGKHLNSMERVEWLRKKLQDVHNFVALGAPLAPRDAGS
(PTH 1-47)
SEQ ID NO: 38
SVSEIQLMHNLGKHLNSMERVEWLRKKLQDVHNFVALGAPLAPRDAG
(PTH 1-46)
SEQ ID NO: 39
SVSEIQLMHNLGKHLNSMERVEWLRKKLQDVHNFVALGAPLAPRDA
(PTH 1-45)
SEQ ID NO: 40
SVSEIQLMHNLGKHLNSMERVEWLRKKLQDVHNFVALGAPLAPRD
(PTH 1-44)
SEQ ID NO: 41
SVSEIQLMHNLGKHLNSMERVEWLRKKLQDVHNFVALGAPLAPR
(PTH 1-43)
SEQ ID NO: 42
SVSEIQLMHNLGKHLNSMERVEWLRKKLQDVHNFVALGAPLAP
(PTH 1-42)
SEQ ID NO: 43
SVSEIQLMHNLGKHLNSMERVEWLRKKLQDVHNFVALGAPLA
(PTH 1-41)
SEQ ID NO: 44
SVSEIQLMHNLGKHLNSMERVEWLRKKLQDVHNFVALGAPL
(PTH 1-40)
SEQ ID NO: 45
SVSEIQLMHNLGKHLNSMERVEWLRKKLQDVHNFVALGAP
(PTH 1-39)
SEQ ID NO: 46
SVSEIQLMHNLGKHLNSMERVEWLRKKLQDVHNFVALGA
(PTH 1-38)
SEQ ID NO: 47
SVSEIQLMHNLGKHLNSMERVEWLRKKLQDVHNFVALG
(PTH 1-37)
SEQ ID NO: 48
SVSEIQLMHNLGKHLNSMERVEWLRKKLQDVHNFVAL
(PTH 1-36)
SEQ ID NO: 49
SVSEIQLMHNLGKHLNSMERVEWLRKKLQDVHNFVA
(PTH 1-35)
SEQ ID NO: 50
SVSEIQLMHNLGKHLNSMERVEWLRKKLQDVHNFV
(PTH 1-34)
SEQ ID NO: 51
SVSEIQLMHNLGKHLNSMERVEWLRKKLQDVHNF
(PTH 1-33)
SEQ ID NO: 52
SVSEIQLMHNLGKHLNSMERVEWLRKKLQDVHN
(PTH 1-32)
SEQ ID NO: 53
SVSEIQLMHNLGKHLNSMERVEWLRKKLQDVH
(PTH 1-31)
SEQ ID NO: 54
SVSEIQLMHNLGKHLNSMERVEWLRKKLQDV
(PTH 1-30)
SEQ ID NO: 55
SVSEIQLMHNLGKHLNSMERVEWLRKKLQD
(PTH 1-29)
SEQ ID NO: 56
SVSEIQLMHNLGKHLNSMERVEWLRKKLQ
(PTH 1-28)
SEQ ID NO: 57
SVSEIQLMHNLGKHLNSMERVEWLRKKL
(PTH 1-27)
SEQ ID NO: 58
SVSEIQLMHNLGKHLNSMERVEWLRKK
(PTH 1-26)
SEQ ID NO: 59
SVSEIQLMHNLGKHLNSMERVEWLRK
(PTH 1-25)
SEQ ID NO: 60
SVSEIQLMHNLGKHLNSMERVEWLR
(amidated PTH 1-84)
SEQ ID NO: 61
SVSEIQLMHNLGKHLNSMERVEWLRKKLQDVHNFVALGAPLAPRDAGSQRPRKKE
DNVLVESHEKSLGEADKADVNVLTKAKSQ;
wherein the C-terminus is amidated
(amidated PTH 1-83)
SEQ ID NO: 62
SVSEIQLMHNLGKHLNSMERVEWLRKKLQDVHNFVALGAPLAPRDAGSQRPRKKE
DNVLVESHEKSLGEADKADVNVLTKAKS;
wherein the C-terminus is amidated
(amidated PTH 1-82)
SEQ ID NO: 63
SVSEIQLMHNLGKHLNSMERVEWLRKKLQDVHNFVALGAPLAPRDAGSQRPRKKE
DNVLVESHEKSLGEADKADVNVLTKAK;
wherein the C-terminus is amidated
(amidated PTH 1-81)
SEQ ID NO: 64
SVSEIQLMHNLGKHLNSMERVEWLRKKLQDVHNFVALGAPLAPRDAGSQRPRKKE
DNVLVESHEKSLGEADKADVNVLTKA;
wherein the C-terminus is amidated
(amidated PTH 1-80)
SEQ ID NO: 65
SVSEIQLMHNLGKHLNSMERVEWLRKKLQDVHNFVALGAPLAPRDAGSQRPRKKE
DNVLVESHEKSLGEADKADVNVLTK;
wherein the C-terminus is amidated
(amidated PTH 1-79)
SEQ ID NO: 66
SVSEIQLMHNLGKHLNSMERVEWLRKKLQDVHNFVALGAPLAPRDAGSQRPRKKE
DNVLVESHEKSLGEADKADVNVLT;
wherein the C-terminus is amidated
(amidated PTH 1-78)
SEQ ID NO: 67
SVSEIQLMHNLGKHLNSMERVEWLRKKLQDVHNFVALGAPLAPRDAGSQRPRKKE
DNVLVESHEKSLGEADKADVNVL;
wherein the C-terminus is amidated
(amidated PTH 1-77)
SEQ ID NO: 68
SVSEIQLMHNLGKHLNSMERVEWLRKKLQDVHNFVALGAPLAPRDAGSQRPRKKE
DNVLVESHEKSLGEADKADVNV;
wherein the C-terminus is amidated
(amidated PTH 1-76)
SEQ ID NO: 69
SVSEIQLMHNLGKHLNSMERVEWLRKKLQDVHNFVALGAPLAPRDAGSQRPRKKE
DNVLVESHEKSLGEADKADVN;
wherein the C-terminus is amidated
(amidated PTH 1-75)
SEQ ID NO: 70
SVSEIQLMHNLGKHLNSMERVEWLRKKLQDVHNFVALGAPLAPRDAGSQRPRKKE
DNVLVESHEKSLGEADKADV;
wherein the C-terminus is amidated
(amidated PTH 1-74)
SEQ ID NO: 71
SVSEIQLMHNLGKHLNSMERVEWLRKKLQDVHNFVALGAPLAPRDAGSQRPRKKE
DNVLVESHEKSLGEADKAD;
wherein the C-terminus is amidated
(amidated PTH 1-73)
SEQ ID NO: 72
SVSEIQLMHNLGKHLNSMERVEWLRKKLQDVHNFVALGAPLAPRDAGSQRPRKKE
DNVLVESHEKSLGEADKA;
wherein the C-terminus is amidated
(amidated PTH 1-72)
SEQ ID NO: 73
SVSEIQLMHNLGKHLNSMERVEWLRKKLQDVHNFVALGAPLAPRDAGSQRPRKKE
DNVLVESHEKSLGEADK;
wherein the C-terminus is amidated
(amidated PTH 1-71)
SEQ ID NO: 74
SVSEIQLMHNLGKHLNSMERVEWLRKKLQDVHNFVALGAPLAPRDAGSQRPRKKE
DNVLVESHEKSLGEAD;
wherein the C-terminus is amidated
(amidated PTH 1-70)
SEQ ID NO: 75
SVSEIQLMHNLGKHLNSMERVEWLRKKLQDVHNFVALGAPLAPRDAGSQRPRKKE
DNVLVESHEKSLGEA;
wherein the C-terminus is amidated
(amidated PTH 1-69)
SEQ ID NO: 76
SVSEIQLMHNLGKHLNSMERVEWLRKKLQDVHNFVALGAPLAPRDAGSQRPRKKE
DNVLVESHEKSLGE;
wherein the C-terminus is amidated
(amidated PTH 1-68)
SEQ ID NO: 77
SVSEIQLMHNLGKHLNSMERVEWLRKKLQDVHNFVALGAPLAPRDAGSQRPRKKE
DNVLVESHEKSLG;
wherein the C-terminus is amidated
(amidated PTH 1-67)
SEQ ID NO: 78
SVSEIQLMHNLGKHLNSMERVEWLRKKLQDVHNFVALGAPLAPRDAGSQRPRKKE
DNVLVESHEKSL;
wherein the C-terminus is amidated
(amidated PTH 1-66)
SEQ ID NO: 79
SVSEIQLMHNLGKHLNSMERVEWLRKKLQDVHNFVALGAPLAPRDAGSQRPRKKE
DNVLVESHEKS;
wherein the C-terminus is amidated
(amidated PTH 1-65)
SEQ ID NO: 80
SVSEIQLMHNLGKHLNSMERVEWLRKKLQDVHNFVALGAPLAPRDAGSQRPRKKE
DNVLVESHEK;
wherein the C-terminus is amidated
(amidated PTH 1-64)
SEQ ID NO: 81
SVSEIQLMHNLGKHLNSMERVEWLRKKLQDVHNFVALGAPLAPRDAGSQRPRKKE
DNVLVESHE;
wherein the C-terminus is amidated
(amidated PTH 1-63)
SEQ ID NO: 82
SVSEIQLMHNLGKHLNSMERVEWLRKKLQDVHNFVALGAPLAPRDAGSQRPRKKE
DNVLVESH;
wherein the C-terminus is amidated
(amidated PTH 1-62)
SEQ ID NO: 83
SVSEIQLMHNLGKHLNSMERVEWLRKKLQDVHNFVALGAPLAPRDAGSQRPRKKE
DNVLVES;
wherein the C-terminus is amidated
(amidated PTH 1-61)
SEQ ID NO: 84
SVSEIQLMHNLGKHLNSMERVEWLRKKLQDVHNFVALGAPLAPRDAGSQRPRKKE
DNVLVE;
wherein the C-terminus is amidated
(amidated PTH 1-60)
SEQ ID NO: 85
SVSEIQLMHNLGKHLNSMERVEWLRKKLQDVHNFVALGAPLAPRDAGSQRPRKKE
DNVLV;
wherein the C-terminus is amidated
(amidated PTH 1-59)
SEQ ID NO: 86
SVSEIQLMHNLGKHLNSMERVEWLRKKLQDVHNFVALGAPLAPRDAGSQRPRKKE
DNVL;
wherein the C-terminus is amidated
(amidated PTH 1-58)
SEQ ID NO: 87
SVSEIQLMHNLGKHLNSMERVEWLRKKLQDVHNFVALGAPLAPRDAGSQRPRKKE
DNV;
wherein the C-terminus is amidated
(amidated PTH 1-57)
SEQ ID NO: 88
SVSEIQLMHNLGKHLNSMERVEWLRKKLQDVHNFVALGAPLAPRDAGSQRPRKKE
DN;
wherein the C-terminus is amidated
(amidated PTH 1-56)
SEQ ID NO: 89
SVSEIQLMHNLGKHLNSMERVEWLRKKLQDVHNFVALGAPLAPRDAGSQRPRKKE
D;
wherein the C-terminus is amidated
(amidated PTH 1-55)
SEQ ID NO: 90
SVSEIQLMHNLGKHLNSMERVEWLRKKLQDVHNFVALGAPLAPRDAGSQRPRKKE;
wherein the C-terminus is amidated
(amidated PTH 1-54)
SEQ ID NO: 91
SVSEIQLMHNLGKHLNSMERVEWLRKKLQDVHNFVALGAPLAPRDAGSQRPRKK;
wherein the C-terminus is amidated
(amidated PTH 1-53)
SEQ ID NO: 92
SVSEIQLMHNLGKHLNSMERVEWLRKKLQDVHNFVALGAPLAPRDAGSQRPRK;
wherein the C-terminus is amidated
(amidated PTH 1-52)
SEQ ID NO: 93
SVSEIQLMHNLGKHLNSMERVEWLRKKLQDVHNFVALGAPLAPRDAGSQRPR;
wherein the C-terminus is amidated
(amidated PTH 1-51)
SEQ ID NO: 94
SVSEIQLMHNLGKHLNSMERVEWLRKKLQDVHNFVALGAPLAPRDAGSQRP;
wherein the C-terminus is amidated
(amidated PTH 1-50)
SEQ ID NO: 95
SVSEIQLMHNLGKHLNSMERVEWLRKKLQDVHNFVALGAPLAPRDAGSQR;
wherein the C-terminus is amidated
(amidated PTH 1-49)
SEQ ID NO: 96
SVSEIQLMHNLGKHLNSMERVEWLRKKLQDVHNFVALGAPLAPRDAGSQ;
wherein the C-terminus is amidated
(amidated PTH 1-48)
SEQ ID NO: 97
SVSEIQLMHNLGKHLNSMERVEWLRKKLQDVHNFVALGAPLAPRDAGS;
wherein the C-terminus is amidated
(amidated PTH 1-47)
SEQ ID NO: 98
SVSEIQLMHNLGKHLNSMERVEWLRKKLQDVHNFVALGAPLAPRDAG;
wherein the C-terminus is amidated
(amidated PTH 1-46)
SEQ ID NO: 99
SVSEIQLMHNLGKHLNSMERVEWLRKKLQDVHNFVALGAPLAPRDA;
wherein the C-terminus is amidated
(amidated PTH 1-45)
SEQ ID NO: 100
SVSEIQLMHNLGKHLNSMERVEWLRKKLQDVHNFVALGAPLAPRD;
wherein the C- terminus is amidated
(amidated PTH 1-44)
SEQ ID NO: 101
SVSEIQLMHNLGKHLNSMERVEWLRKKLQDVHNFVALGAPLAPR;
wherein the C-terminus is amidated
(amidated PTH 1-43)
SEQ ID NO: 102
SVSEIQLMHNLGKHLNSMERVEWLRKKLQDVHNFVALGAPLAP;
wherein the C-terminus is is amidated
(amidated PTH 1-42)
SEQ ID NO: 103
SVSEIQLMHNLGKHLNSMERVEWLRKKLQDVHNFVALGAPLA;
wherein the C-terminus is is amidated
(amidated PTH 1-41)
SEQ ID NO: 104
SVSEIQLMHNLGKHLNSMERVEWLRKKLQDVHNFVALGAPL;
wherein the C-terminus is amidated
(amidated PTH 1-40)
SEQ ID NO: 105
SVSEIQLMHNLGKHLNSMERVEWLRKKLQDVHNFVALGAP;
wherein the C-terminus is amidated
(amidated PTH 1-39)
SEQ ID NO: 106
SVSEIQLMHNLGKHLNSMERVEWLRKKLQDVHNFVALGA;
wherein the C-terminus is amidated
(amidated PTH 1-38)
SEQ ID NO: 107
SVSEIQLMHNLGKHLNSMERVEWLRKKLQDVHNFVALG;
wherein the C-terminus is amidated
(amidated PTH 1-37)
SEQ ID NO: 108
SVSEIQLMHNLGKHLNSMERVEWLRKKLQDVHNFVAL;
wherein the C-terminus is amidated
(amidated PTH 1-36)
SEQ ID NO: 109
SVSEIQLMHNLGKHLNSMERVEWLRKKLQDVHNFVA;
wherein the C-terminus is amidated
(amidated PTH 1-35)
SEQ ID NO: 110
SVSEIQLMHNLGKHLNSMERVEWLRKKLQDVHNFV;
wherein the C-terminus is amidated
(amidated PTH 1-34)
SEQ ID NO: 111
SVSEIQLMHNLGKHLNSMERVEWLRKKLQDVHNF;
wherein the C-terminus is amidated
(amidated PTH 1-33)
SEQ ID NO: 112
SVSEIQLMHNLGKHLNSMERVEWLRKKLQDVHN;
wherein the C-terminus is amidated
(amidated PTH 1-32)
SEQ ID NO: 113
SVSEIQLMHNLGKHLNSMERVEWLRKKLQDVH;
wherein the C-terminus is amidated
(amidated PTH 1-31)
SEQ ID NO: 114
SVSEIQLMHNLGKHLNSMERVEWLRKKLQDV;
wherein the C-terminus is amidated
(amidated PTH 1-30)
SEQ ID NO: 115
SVSEIQLMHNLGKHLNSMERVEWLRKKLQD;
wherein the C-terminus is amidated
(amidated PTH 1-29)
SEQ ID NO: 116
SVSEIQLMHNLGKHLNSMERVEWLRKKLQ;
wherein the C-terminus is amidated
(amidated PTH 1-28)
SEQ ID NO: 117
SVSEIQLMHNLGKHLNSMERVEWLRKKL;
wherein the C-terminus is amidated
(amidated PTH 1-27)
SEQ ID NO: 118
SVSEIQLMHNLGKHLNSMERVEWLRKK;
wherein the C-terminus is amidated
(amidated PTH 1-26)
SEQ ID NO: 119
SVSEIQLMHNLGKHLNSMERVEWLRK;
wherein the C-terminus is amidated
(amidated PTH 1-25)
SEQ ID NO: 120
SVSEIQLMHNLGKHLNSMERVEWLR;
wherein the C-terminus is amidated
(PTHrP)
SEQ ID NO: 121
AVSEHQLLHDKGKSIQDLRRRFFLHHLIAEIHTAEIRATSEVS
PNSKPSPNTKNHPVRFGSDDEGRYLTQETNKVETYKEQPLKTP
GKKKKGKPGKRKEQEKKKRRTRSAWLDSGVTGSGLEGDHLSDT
STTSLELDSRRH

and sequences having at least 90%, such as at least 91%, at least 92%, at least 93%, at least 94%, at least 95%, at least 96%, at least 97%, at least 98% or at least 99% homology thereto.

The terms “PTH molecule” and “PTH moiety” also include poly(amino acid) conjugates which have a sequence as described above, but having a backbone that comprises both amide and non-amide linkages, such as ester linkages, like for example depsipeptides. Depsipeptides are chains of amino acid residues in which the backbone comprises both amide (peptide) and ester bonds. Accordingly, the term “side chain” as used herein refers either to the moiety attached to the alpha-carbon of an amino acid moiety, if the amino acid moiety is connected through amine bonds such as in proteins and peptides, or to any carbon atom-comprising moiety attached to the backbone of a poly(amino acid) conjugate, such as for example in the case of depsipeptides.

In certain embodiments the term “PTH” refers to sequences having a backbone formed through amide (peptide) bonds.

As used herein the term “long-acting PTH compound” refers to a compound comprising a PTH molecule or PTH moiety that is capable of maintaining a PD response, such as increasing serum calcium levels, for at least 24 h following administration.

As used herein the term “sustained-release PTH compound” refers to any compound, conjugate, crystal or admixture that comprises at least one PTH molecule or PTH moiety and from which the at least one PTH molecule or PTH moiety is released with a release half-life of at least 12 hours.

As used herein the terms “release half-life” and “half-life” refer to the time required under physiological conditions (i.e. aqueous buffer, pH 7.4, 37° C.) until half of all PTH molecules or PTH moieties, respectively, of a sustained-release PTH compound are released.

The term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties. As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties. For simplification, PTH moieties and PTH molecules are generally referred to herein as “polypeptides”.

As used herein the term “physiological conditions” refers to aqueous buffer at pH 7.4, 37° C.

As used herein the term “pharmaceutical composition” refers to a composition containing one or more active ingredients, such as for example at least one long-acting PTH compound, and one or more excipients, as well as any product which results, directly or indirectly, from combination, complexation or aggregation of any two or more of the ingredients of the composition, or from dissociation of one or more of the ingredients, or from other types of reactions or interactions of one or more of the ingredients. Accordingly, a pharmaceutical composition for use of the present invention encompasses any composition made by admixing one or more long-acting PTH compound and a pharmaceutically acceptable excipient.

As used herein, the term “excipient” refers to a diluent, adjuvant, or vehicle with which the therapeutic, such as a drug or prodrug, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is an example for an excipient when the pharmaceutical composition is administered orally. Saline and aqueous dextrose are examples of excipients when the pharmaceutical composition is administered intravenously. Saline solutions and aqueous dextrose and glycerol solutions are in certain embodiments employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, tale, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, or sustained-release formulations. The pharmaceutical composition may be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or biologically active moiety, together with a suitable amount of excipient so as to provide the form for proper administration to the patient. The formulation should suit the mode of administration.

As used herein the term “liquid composition” refers to a mixture comprising a water-soluble long-acting PTH compound and one or more solvents, such as water.

The term “suspension composition” relates to a mixture comprising at least one water-insoluble long-acting PTH compound and one or more solvents, such as water.

As used herein, the term “dry composition” means that a pharmaceutical composition is provided in a dry form. Suitable methods for drying are spray-drying and lyophilization, i.e. freeze-drying. Such dry composition has a residual water content of a maximum of 10%, such as less than 5% or less than 2%, determined according to Karl Fischer. In certain embodiments such dry pharmaceutical composition is dried by lyophilization.

The term “drug” as used herein refers to a substance, such as PTH, used in the treatment, cure, prevention, or diagnosis of a disease or used to otherwise enhance physical or mental well-being. If a drug is conjugated to another moiety, the moiety of the resulting product that originated from the drug is referred to as “drug moiety”.

As used herein the term “prodrug” refers to a covalent conjugate in which a drug moiety is reversibly and covalently connected to a specialized protective group through a reversible linker moiety, also referred to as “reversible prodrug linker moiety” or “reversible linker moiety”, which is conjugated through a reversible linkage to the biologically active moiety and wherein the specialized protective group alters or eliminates undesirable properties in the parent molecule. This also includes the enhancement of desirable properties in the drug and the suppression of undesirable properties. The specialized non-toxic protective group is referred to as “carrier”. A prodrug releases the reversibly and covalently bound drug moiety in the form of its corresponding drug. In other words, a prodrug is a conjugate comprising a drug moiety which is covalently and reversibly conjugated to a carrier moiety via a reversible linker moiety, which covalent and reversible conjugation of the carrier to the reversible linker moiety is either directly or through a spacer. Such conjugate releases the formerly conjugated drug moiety in the form of a free unmodified drug.

A “biodegradable linkage” or a “reversible linkage” is a linkage that is hydrolytically degradable, i.e. cleavable, in the absence of enzymes under physiological conditions (aqueous buffer at pH 7.4, 37° C.) with a half-life ranging from one hour to three months, in certain embodiments from one hour to two months, in certain embodiments from one hour to one month, in certain embodiments from one hour to three weeks, in certain embodiments from one hour to two weeks, in certain embodiments from 12 hours to two weeks, in certain embodiments from 12 hours to one week. Accordingly, a stable linkage is a linkage having a half-life under physiological conditions (aqueous buffer at pH 7.4, 37° C.) of more than three months.

As used herein, the terms “traceless prodrug linker” or “traceless linker” means a reversible prodrug linker, i.e. a linker moiety reversibly and covalently connecting the drug moiety with the carrier, which upon cleavage releases the drug in its free form. As used herein, the term “free form” of a drug means the drug in its unmodified, pharmacologically active form.

As used herein, the term “reagent” means a chemical compound which comprises at least one functional group for reaction with the functional group of another chemical compound or drug.

It is understood that a drug comprising a functional group, such as a primary or secondary amine or hydroxyl functional group is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “—” indicates attachment to another moiety. Accordingly, a drug moiety is released from a prodrug as a drug.

It is understood that if the chemical structure of a group of atoms is provided which group of atoms is attached to at least one other moiety or is interrupting a moiety, said chemical structure may be attached to the at least one further or interrupted moiety in either orientation, unless explicitly stated otherwise. For example, a moiety “—C(O)N(R¹)—” may be attached to two moieties or interrupting a moiety either as “—C(O)N(R¹)—” or as “—N(R¹)C(O)—”. Similarly, a moiety

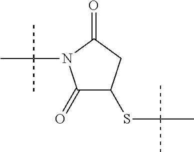

may be attached to two moieties or can interrupt a moiety either as

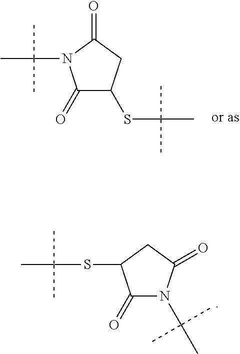

As used herein, the term “functional group” means a group of atoms which can react with other groups of atoms. Functional groups include but are not limited to the following groups: carboxylic acid, primary or secondary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case the long-acting PTH compound comprises one or more acidic or basic groups, the invention also comprises their corresponding pharmaceutically or toxicologically acceptable salts, in particular their pharmaceutically utilizable salts. Thus, the long-acting PTH compound comprising acidic groups may be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. A long-acting PTH compound comprising one or more basic groups, i.e. groups which can be protonated, may be present and may be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. For the person skilled in the art further methods are known for converting the basic group into a cation like the alkylation of an amine group resulting in a positively-charge ammonium group and an appropriate counterion of the salt. If the long-acting PTH compound simultaneously comprises acidic and basic groups, the invention also includes, in addition to the salt forms mentioned above, inner salts or betaines (zwitterions). The respective salts may be obtained by customary methods which are known to the person skilled in the art like, for example by contacting these compounds with an organic or inorganic acid or base in a solvent or dispersant, or by anion exchange or cation exchange with other salts. The present invention also includes all salts of the compounds which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and in certain embodiments means approved by a regulatory agency, such as the EMA (Europe) and/or the FDA (US) and/or any other national regulatory agency for use in animals, in particular for use in humans.

As used herein the term “about” in combination with a numerical value is used to indicate a range ranging from and including the numerical value plus and minus no more than 10% of said numerical value, in certain embodiments no more than 8% of said numerical value, in certain embodiments no more than 5% of said numerical value and in certain embodiments no more than 2% of said numerical value. For example, the phrase “about 200” is used to mean a range ranging from and including 200+/−10%, i.e. ranging from and including 180 to 220; in certain embodiments 200+/−8%, i.e. ranging from and including 184 to 216; in certain embodiments ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. It is understood that a polymer may also comprise one or more other chemical groups and/or moieties, such as, for example, one or more functional groups. In certain embodiments a soluble polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. If the polymer is soluble, it in certain embodiments has a molecular weight of at most 1000 kDa, such as at most 750 kDa, such as at most 500 kDa, such as at most 300 kDa, such as at most 200 kDa, such as at most 100 kDa. It is understood that for water-insoluble polymers, such as hydrogels, no meaningful molecular weight ranges can be provided. It is understood that also a peptide or protein is a polymer in which the amino acids are the repeating structural units, even though the side chains of each amino acid may be different.

As used herein, the term “polymeric” means a reagent or a moiety comprising one or more polymers or polymer moieties. A polymeric reagent or moiety may optionally also comprise one or more other moiety/moieties, which are in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

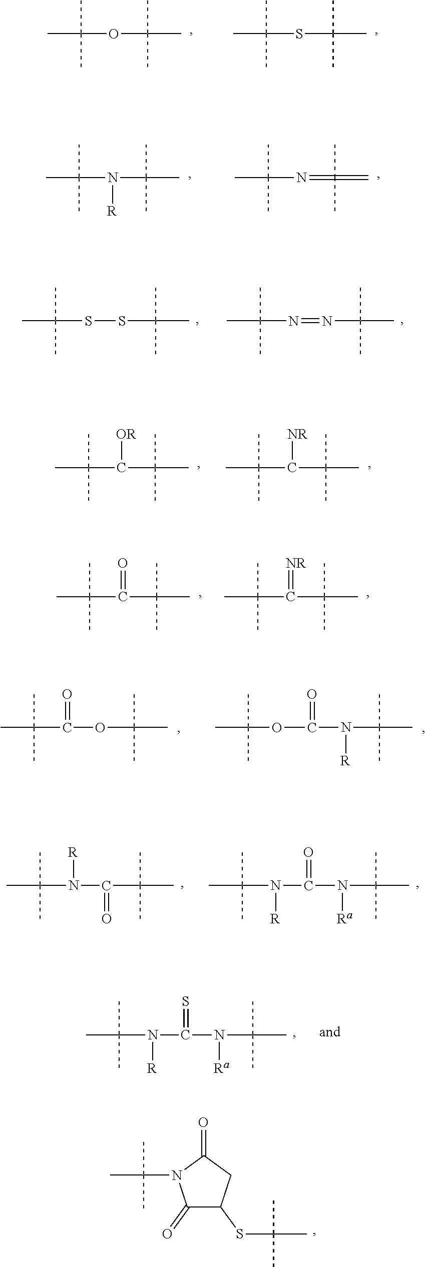

wherein
dashed lines indicate attachment to the remainder of the moiety or reagent, and
—R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−10%, in certain embodiments x+/−8%, in certain embodiments x+/−5% and in certain embodiments x+/−2%.

As used herein, the term “number average molecular weight” means the ordinary arithmetic mean of the molecular weights of the individual polymers.

As used herein the term “water-soluble” with reference to the long-acting PTH compound means that at least 1 g of the long-acting PTH compound may be dissolved in one liter of water at 20° C. to form a homogeneous solution. Accordingly, the term “water-insoluble” with reference to the long-acting PTH compound means that less than 1 g of the long-acting PTH compound may be dissolved in one liter of water at 20° C. to form a homogeneous solution.

As used herein, the term “PEG-based” in relation to a moiety or reagent means that said moiety or reagent comprises PEG. In certain embodiments a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, such as at least 95%. The remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

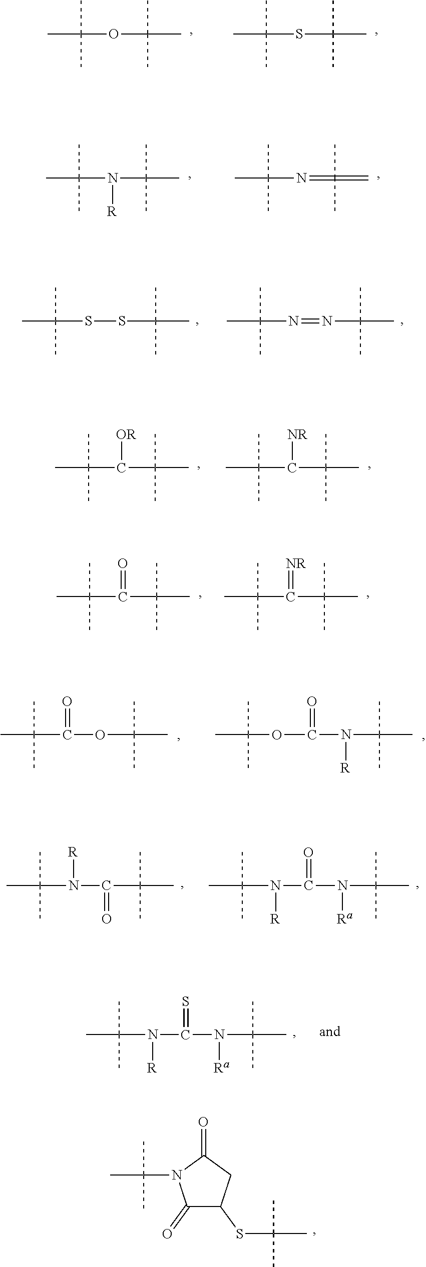

wherein
dashed lines indicate attachment to the remainder of the moiety or reagent, and
—R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The term “substituted” as used herein means that one or more —H atom(s) of a molecule or moiety are replaced by a different atom or a group of atoms, which are referred to as “substituent”.

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
—R^{x1}, —R^{x1a}, —R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{4b} is independently selected from the group consisting of —H and C₁₋₆ alkyl; wherein C₁₋₆ alkyl is optionally substituted with one or more halogen, which are the same or different.

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR⁴, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl;
In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —N02, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b})—;
—OC(O)N(R^{x1}R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x2}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

The term “interrupted” means that a moiety is inserted between two carbon atoms or—if the insertion is at one of the moiety's ends—between a carbon or heteroatom and a hydrogen atom, in certain embodiments between a carbon and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₁₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

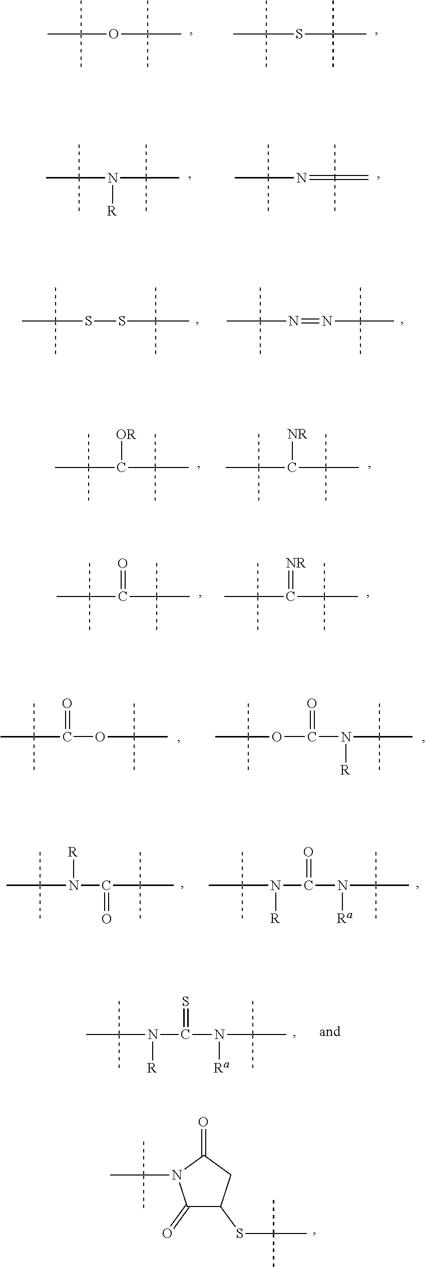

wherein
dashed lines indicate attachment to the remainder of the moiety or reagent; and
—R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments an 8- to 30-membered carbopolycyclyl means a cyclic moiety of two, three, four or five rings, in certain embodiments of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)— and —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

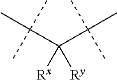

means that R^{x} and R^{y} form the following structure:

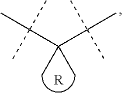

wherein R is C₃₋₁₀ cycloalkyl or 3- to 10-membered heterocyclyl.

It is also understood that the phrase “the pair R^{x}/R^{y} is joint together with the atoms to which they are attached to form a ring A” in relation with a moiety of the structure

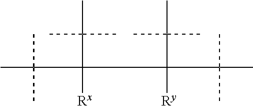

means that R^{x} and R^{y} form the following structure:

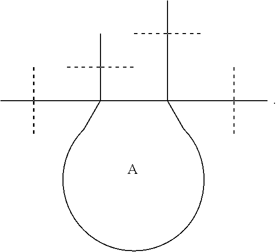

As used herein, “halogen” means fluoro, chloro, bromo or iodo. In certain embodiments halogen is fluoro or chloro.

In general, the term “comprise” or “comprising” also encompasses “consist of” or “consisting of”.

In certain embodiments BMD is reduced within 58 weeks of beginning administration of the long-acting PTH compound.

In certain embodiments the use of the first aspect comprises monitoring the BMD and thereby determining BMD has been reduced. In certain embodiments such monitoring comprises performing DXA-scans on the patient and determining from the results of such DXA-scans that the BMD has been reduced.

In certain embodiments BMD is reduced within 58 weeks of beginning administration of the long-acting PTH compound.

In certain embodiments the use of the first aspect is performed on a population of patients having hypoparathyroidism, wherein the population shows a statistically significant improvement in BMD relative to a control population not receiving the long-acting PTH compound. In certain embodiments the control population is a healthy population that is matched for gender, ethnicity and age. In certain embodiments the statistically significant improvement is detected by 58 weeks of initiating the administration of the long-acting PTH compound.

In certain embodiments the long-acting PTH compound for use of the first aspect is administered in a treatment that comprises the steps of (a) administering to said patient a pharmaceutically effective dose of the long-acting PTH compound over a certain period of time, (b) measuring BMD, and optionally (c) of adjusting the dose depending on BMD. Steps (b) and (c) may be repeated.

In certain embodiments the period of time for which the long-acting PTH compound is administered to the patient in step (a) is at least one week, at least two weeks, at least three weeks, at least four weeks, at least six weeks, at least eight weeks or at least ten weeks.

In certain embodiments the BMD in step (b) is measured by DXA.

In certain embodiments the adjustment of the dose in step (c) is an increase in the dose, such as by a factor of 1.1, 1.2, 1.3, 1.4, 1.5, 1.6, 1.7, 1.8, 1.9 or 2, when no or insufficient reduction in BMD is observed. If step (b) indicates a suitable rate in reduction of BMD, no dose adjustment is required.

If the patient having an increased BMD of the first aspect is a patient having hypoparathyroidism, the effective dose of the long-acting PTH compound in step (a) is a dose that results in serum calcium levels within the normal range in the patient. In certain embodiments the starting dose for step (a) ranges from 3 to 5 nmol PTH/day or from 4 to 5 nmol PTH/day. In certain embodiments the starting dose is 4.4±0.3 nmol PTH/day. In certain embodiments the starting dose is 4.4±0.2 nmol PTH/day. In certain embodiments the starting dose is 4.4±0.1 nmol PTH/day.

In certain embodiments the long-acting PTH compound may in certain embodiments be administered to a patient having hypoparathyroidism in a dosage regimen comprising the steps of
(i) titrating the dose of the long-acting PTH compound administered to the patient to result in normal serum calcium levels in the patient and maintaining the patient on such dose for a first time period;
(ii) increasing the dose of the long-acting PTH compound for a second time period directly following the first time period by a factor of at least 1.1; and
(iii) optionally increasing the dose of the long-acting PTH compound for a third or further subsequent time period by a factor of at least 1.1.

It is understood that for each of the third or further subsequent time periods the dose of the long-acting PTH compound is increased by a factor of at least 1.1 compared to the dose administered in the period directly preceding it.

In certain embodiments the first time period of the first aspect is at least two weeks. In certain embodiments the first time period of the first aspect is at least 1 month. In certain embodiments the first time period of the first aspect is at least 2 months. In certain embodiments the first time period of the first aspect is at least 3 months. In certain embodiments the first time period of the first aspect is at least 4 months. In certain embodiments the first time period of the first aspect is at least 5 months. In certain embodiments the first time period of the first aspect is at least 6 months. In certain embodiments the first time period of the first aspect is at least 7 months. In certain embodiments the first time period of the first aspect is at least 8 months.

In certain embodiments the second time period of the first aspect is at least two weeks. In certain embodiments the second time period of the first aspect is at least 1 month. In certain embodiments the second time period of the first aspect is at least 2 months. In certain embodiments the second time period of the first aspect is at least 3 months. In certain embodiments the second time period of the first aspect is at least 4 months.

In certain embodiments the factor in step (ii) of the first aspect is at least 1.2. In certain embodiments the factor in step (ii) of the first aspect is at least 1.3. In certain embodiments the factor in step (ii) of the first aspect is at least 1.4. In certain embodiments the factor in step (ii) of the first aspect is at least 1.5.

In certain embodiments the factor in step (iii) of the first aspect is at least 1.2. In certain embodiments the factor in step (iii) of the first aspect is at least 1.4. In certain embodiments the factor in step (iii) of the first aspect is at least 1.3. In certain embodiments the factor in step (iii) of the first aspect is at least 1.4.

In certain embodiments the dose administered to the patient increases in the course of the treatment due to increasing physical activity of the patient.

In a second aspect the present invention relates to a method of reducing bone mineral density in a subject having increased BMD, the method comprising the step of administering to said patient a pharmaceutically effective dose of a long-acting PTH compound. It is understood that the term “subject” and “patient” can be used interchangeably.

In certain embodiments the method of the second aspect further comprises monitoring the BMD and thereby determining BMD has been reduced. In certain embodiments such monitoring comprises performing DXA-scans on the patient and determining from the results of such DXA-scans that the BMD has been reduced.

In certain embodiments BMD is reduced within 58 weeks of beginning administration of the long-acting PTH compound.

In certain embodiments the method of the third aspect is performed on a population of patients having hypoparathyroidism, wherein the population shows a statistically significant improvement in BMD relative to a control population not receiving the long-acting PTH compound. In certain embodiments the control population is a healthy population that is matched for gender, ethnicity and age. In certain embodiments the statistically significant improvement is detected by 58 weeks of initiating the administration of the long-acting PTH compound.

In certain embodiments the method of the second aspect comprises the steps of (a) administering to said patient a pharmaceutically effective dose of the long-acting PTH compound over a certain period of time, (b) measuring BMD, and optionally (c) of adjusting the dose depending on BMD. Steps (b) and (c) may be repeated.

In certain embodiments the period of time for which the long-acting PTH compound is administered to the patient in step (a) is at least one week, at least two weeks, at least three weeks, at least four weeks, at least six weeks, at least eight weeks or at least ten weeks.

In certain embodiments the BMD in step (b) is measured by DXA.

In certain embodiments the adjustment of the dose in step (c) is an increase in the dose, such as by a factor of 1.1, 1.2, 1.3, 1.4, 1.5, 1.6, 1.7, 1.8, 1.9 or 2, when no or insufficient reduction in BMD is observed. If step (b) indicates a suitable rate in reduction of BMD, no dose adjustment is required.

If the patient having an increased BMD of the second aspect is a patient having hypoparathyroidism, the effective dose of the long-acting PTH compound in step (a) is a dose that results in serum calcium levels within the normal range in the patient. In certain embodiments the starting dose for step (a) ranges from 3 to 5 nmol PTH/day or from 4 to 5 nmol PTH/day. In certain embodiments the starting dose is 4.4±0.3 nmol PTH/day. In certain embodiments the starting dose is 4.4±0.2 nmol PTH/day. In certain embodiments the starting dose is 4.4±0.1 nmol PTH/day.

If the patient having an increased BMD of the second aspect is a patient having hypoparathyroidism, the method may in certain embodiments comprise the steps of
(i) titrating the dose of the long-acting PTH compound administered to the patient to result in normal serum calcium levels in the patient and maintaining the patient on such dose for a first time period;
(ii) increasing the dose of the long-acting PTH compound for a second time period directly following the first time period by a factor of at least 1.1; and
(iii) optionally increasing the dose of the long-acting PTH compound for a third or further subsequent time period by a factor of at least 1.1.

It is understood that for each of the third or further subsequent time periods the dose of the long-acting PTH compound is increased by a factor of at least 1.1 compared to the dose administered in the period directly preceding it.

In certain embodiments the first time period of the second aspect is at least two weeks. In certain embodiments the first time period of the second aspect is at least 1 month. In certain embodiments the first time period of the second aspect is at least 2 months. In certain embodiments the first time period of the second aspect is at least 3 months. In certain embodiments the first time period of the second aspect is at least 4 months. In certain embodiments the first time period of the second aspect is at least 5 months. In certain embodiments the first time period of the first second is at least 6 months. In certain embodiments the first time period of the second aspect is at least 7 months. In certain embodiments the first time period of the second aspect is at least 8 months.

In certain embodiments the second time period of the second aspect is at least two weeks. In certain embodiments the second time period of the second aspect is at least 1 month. In certain embodiments the second time period of the second aspect is at least 2 months. In certain embodiments the second time period of the second aspect is at least 3 months. In certain embodiments the second time period of the second aspect is at least 4 months.

In certain embodiments the factor in step (ii) of the second aspect is at least 1.2. In certain embodiments the factor in step (ii) of the second aspect is at least 1.3. In certain embodiments the factor in step (ii) of the second aspect is at least 1.4. In certain embodiments the factor in step (ii) of the second aspect is at least 1.5.

In certain embodiments the factor in step (iii) of the second aspect is at least 1.2. In certain embodiments the factor in step (iii) of the second aspect is at least 1.4. In certain embodiments the factor in step (iii) of the second aspect is at least 1.3. In certain embodiments the factor in step (iii) of the first aspect is at least 1.4.

In certain embodiments the dose administered to the patient increases in the course of the treatment due to increasing physical activity of the patient.

In a third aspect the present invention relates to a method of reducing BMD in a patient having increased BMD, comprising administering to the patient a long-acting PTH compound, wherein the long-acting PTH compound reduces the BMD of the patient.

In certain embodiments the method further comprises monitoring the BMD and thereby determining BMD has been reduced. In certain embodiments such monitoring comprises performing DXA-scans on the patient and determining from the results of such DXA-scans that the BMD has been reduced.

In certain embodiments BMD is reduced within 58 weeks of beginning administration of the long-acting PTH compound.

In certain embodiments the method is performed on a population of patients having hypoparathyroidism, wherein the population shows a statistically significant improvement in BMD relative to a control population not receiving the long-acting PTH compound. In certain embodiments the control population is a healthy population that is matched for gender, ethnicity and age. In certain embodiments the statistically significant improvement is detected by 58 weeks of initiating the administration of the long-acting PTH compound.

In certain embodiments the long-acting PTH compound in the method of the third aspect is administered in a treatment that comprises the steps of (a) administering to said patient a pharmaceutically effective dose of the long-acting PTH compound over a certain period of time, (b) measuring BMD, and optionally (c) of adjusting the dose depending on BMD. Steps (b) and (c) may be repeated.

In certain embodiments the period of time for which the long-acting PTH compound is administered to the patient in step (a) is at least one week, at least two weeks, at least three weeks, at least four weeks, at least six weeks, at least eight weeks or at least ten weeks.

In certain embodiments the BMD in step (b) is measured by DXA.

In certain embodiments the adjustment of the dose in step (c) is an increase in the dose, such as by a factor of 1.1, 1.2, 1.3, 1.4, 1.5, 1.6, 1.7, 1.8, 1.9 or 2, when no or insufficient reduction in BMD is observed. If step (b) indicates a suitable rate in reduction of BMD, no dose adjustment is required.

If the patient having an increased BMD of the third aspect is a patient having hypoparathyroidism, the effective dose of the long-acting PTH compound in step (a) is a dose that results in serum calcium levels within the normal range in the patient. In certain embodiments the starting dose for step (a) ranges from 3 to 5 nmol PTH/day or from 4 to 5 nmol PTH/day. In certain embodiments the starting dose is 4.4±0.3 nmol PTH/day. In certain embodiments the starting dose is 4.4±0.2 nmol PTH/day. In certain embodiments the starting dose is 4.4±0.1 nmol PTH/day.

If the patient having an increased BMD of the third aspect is a patient having hypoparathyroidism, the method may in certain embodiments comprise the steps of
(i) titrating the dose of the long-acting PTH compound administered to the patient to result in normal serum calcium levels in the patient and maintaining the patient on such dose for a first time period;
(ii) increasing the dose of the long-acting PTH compound for a second time period directly following the first time period by a factor of at least 1.1; and
(iii) optionally increasing the dose of the long-acting PTH compound for a third or further subsequent time period by a factor of at least 1.1.

It is understood that for each of the third or further subsequent time periods the dose of the long-acting PTH compound is increased by a factor of at least 1.1 compared to the dose administered in the period directly preceding it.

In certain embodiments the first time period of the third aspect is at least two weeks. In certain embodiments the first time period of the third aspect is at least 1 month. In certain embodiments the first time period of the third aspect is at least 2 months. In certain embodiments the first time period of the third aspect is at least 3 months. In certain embodiments the first time period of the third aspect is at least 4 months. In certain embodiments the first time period of the third aspect is at least 5 months. In certain embodiments the first time period of the third aspect is at least 6 months. In certain embodiments the first time period of the third aspect is at least 7 months. In certain embodiments the first time period of the third aspect is at least 8 months.

In certain embodiments the second time period of the third aspect is at least two weeks. In certain embodiments the second time period of the third aspect is at least 1 month. In certain embodiments the second time period of the third aspect is at least 2 months. In certain embodiments the second time period of the third aspect is at least 3 months. In certain embodiments the second time period of the third aspect is at least 4 months.

In certain embodiments the factor in step (ii) of the third aspect is at least 1.2. In certain embodiments the factor in step (ii) of the third aspect is at least 1.3. In certain embodiments the factor in step (ii) of the third aspect is at least 1.4. In certain embodiments the factor in step (ii) of the third aspect is at least 1.5.

In certain embodiments the factor in step (iii) of the third aspect is at least 1.2. In certain embodiments the factor in step (iii) of the third aspect is at least 1.4. In certain embodiments the factor in step (iii) of the third aspect is at least 1.3. In certain embodiments the factor in step (iii) of the third aspect is at least 1.4.

In certain embodiments the dose administered to the patient increases in the course of the treatment due to increasing physical activity of the patient.

In certain embodiments the patient having increased BMD in the first to third aspect is a patient having a disease selected from the group consisting of hypoparathyroidism; SAPHO (synovitis, acne, pustulosis, hyperostosis, osteitis) syndrome; chronic infective osteomyelitis; osseous tuberous sclerosis; fluorosis; renal osteodystrophy; acromegaly; hepatitis C-associated osteosclerosis; myelofibrosis; mastocytosis; congenital conditions of reduced bone resorption such as osteopetrosis, pycnodysostosis, osteopoikilosis and melorheostosis; congenital conditions of increased bone formation such as sclerosteosis, van Buchem's disease, LRP5 HBM, LRP4 HBM, craniometaphyseal dysplasia; and conditions of disturbed formation and resorption such as Camurati Engelmann disease and Ghosal syndrome.

In certain embodiments the patient having increased BMD in the first to third aspect suffers from hypoparathyroidism. In certain embodiments the patient having increased BMD in the first to third aspect suffers from osteopetrosis.

In a fourth aspect the present invention relates to a method for reducing BMD of a patient having hypoparathyroidism, comprising administering to the patient a regime of a long-acting PTH compound; monitoring whether BMD has improved; and adjusting the regime depending on presence and extend of improvement in BMD. In certain embodiments the adjustment is a change in dosage of the long-acting PTH compound. In certain embodiments such monitoring comprises performing DXA-scans on the patient and determining from the results of such DXA-scans that the BMD has been reduced.

In certain embodiments BMD is reduced within 58 weeks of beginning administration of the long-acting PTH compound.

In certain embodiments the method of the fourth aspect is performed on a population of patients having hypoparathyroidism, wherein the population shows a statistically significant improvement in BMD relative to a control population not receiving the long-acting PTH compound. In certain embodiments the control population is a healthy population that is matched for gender, ethnicity and age. In certain embodiments the statistically significant improvement is detected by 58 weeks of initiating the administration of the long-acting PTH compound.

In certain embodiments the reduction in BMD in the first to fourth aspect is a reduction in the Z-score of at least 0.1. In certain embodiments the reduction in BMD in the first to fourth aspect is a reduction in the Z-score of at least 0.2. In certain embodiments the reduction in BMD in the first to fourth aspect is a reduction in the Z-score of at least 0.3. In certain embodiments the reduction in BMD in the first to fourth aspect is a reduction in the Z-score of at least 0.4. In certain embodiments the reduction in BMD in the first to fourth aspect is a reduction in the Z-score of at least 0.5. In certain embodiments the reduction in BMD in the first to fourth aspect is a reduction in the Z-score of at least 0.6. In certain embodiments the reduction in BMD in the first to fourth aspect is a reduction in the Z-score of at least 0.7. In certain embodiments the reduction in BMD in the first to fourth aspect is a reduction in the Z-score of at least 0.8. In certain embodiments the reduction in BMD does not result in a Z-score below 0. In certain embodiments the reduction in BMD is achieved 58 weeks after beginning of the treatment. In certain embodiments patients with longer duration of hypoparathyroidism have larger numeric decreases in Z-scores compared to patients with a shorter duration of hypoparathyroidism, in particular at axial sites, e.g. patients with a duration of hypoparathyroidism of more than 10 years have an at least 1.5-fold higher decrease or at least 2-fold higher decrease in Z-scores compared to patients with a duration of hypoparathyroidism of less than 5 years.

In certain embodiments the reduction in BMD in the first to fourth aspect is a reduction of BMD in trabecular bones. It is understood that it may not be feasible to measure the BMD in all trabecular bones, so in certain embodiments the reduction in BMD is measured in lumbar spine L1 to L4, femoral neck and total hip. In certain embodiments the reduction in BMD is measured in at least one region selected from the group consisting of lumbar spine L1 to L4, femoral neck and total hip. In certain embodiments the reduction in BMD is a reduction in BMD in at least one region selected from the group consisting of lumbar spine L1 to L4, femoral neck and total hip. If the patient having increased BMD has such BMD in cortical bones, the reduction in BMD may also be a reduction in cortical bone. In certain embodiments no reduction in BMD is observed in the distal 1/3 radius.

In certain embodiments the reduction in BMD is well tolerated. The phrase “well tolerated” means that no serious treatment-related TEAEs occur. The abbreviation “TEAE” stands for “treatment-emergent adverse events” and refers to undesirable events not present prior to medical treatment, or which events are already present that worsen either in intensity or frequency following the treatment. The term “treatment-related TEAE” refers to a TEAE with a suspected causal relationship to a particular treatment. A “serious treatment-related TEAE” is a treatment-related TEAE that results in death, hospitalization or prolongation of existing hospitalization, persistent or significant disability/incapacity or a congenital anomaly or birth defect.

In a fifth aspect the present invention relates to a long-acting PTH compound for use in a method of treating hypoparathyroidism, wherein the long-acting PTH compound is administered in a dosage regimen, in which the dose of the long-acting PTH compound is increased in the course of the treatment and wherein such dosage regimen comprises the steps of
(i) titrating the dose of the long-acting PTH compound administered to the patient to result in normal serum calcium levels in the patient and maintaining the patient on such dose for a first time period;
(ii) increasing the dose of the long-acting PTH compound for a second time period directly following the first time period by a factor of at least 1.1; and
(iii) optionally increasing the dose of the long-acting PTH compound for a third or further subsequent time period by a factor of at least 1.1.

In a sixth aspect the present invention relates to a method of treating hypoparathyroidism, wherein the method comprises administering to a patient suffering from hypoparathyroidism at least one long-acting PTH compound in a dosage regimen, in which the dose of the long-acting PTH compound is increased in the course of the treatment and wherein such dosage regimen comprises the steps of
(i) titrating the dose of the long-acting PTH compound administered to the patient to result in normal serum calcium levels in the patient and maintaining the patient on such dose for a first time period;
(ii) increasing the dose of the long-acting PTH compound for a second time period directly following the first time period by a factor of at least 1.1; and
(iii) optionally increasing the dose of the long-acting PTH compound for a third or further subsequent time period by a factor of at least 1.1.

It was surprisingly found that patients with hypoparathyroidism require increasing doses of the long-acting PTH compound to maintain normal serum calcium levels.

In certain embodiments the first time period of the fifth and sixth aspect is at least 1 month. In certain embodiments the first time period of the fifth and sixth aspect is at least 2 months. In certain embodiments the first time period of the fifth and sixth aspect is at least 3 months. In certain embodiments the first time period of the fifth and sixth aspect is at least 4 months. In certain embodiments the first time period of the fifth and sixth aspect is at least 5 months. In certain embodiments the first time period of the fifth and sixth aspect is at least 6 months. In certain embodiments the first time period of the fifth and sixth aspect is at least 7 months. In certain embodiments the first time period of the fifth and sixth aspect is at least 8 months.

In certain embodiments the second time period of the fifth and sixth aspect is at least 1 month. In certain embodiments the second time period of the fifth and sixth aspect is at least 2 months. In certain embodiments the second time period of the fifth and sixth aspect is at least 3 months. In certain embodiments the second time period of the fifth and sixth aspect is at least 4 months.

In certain embodiments the factor in step (ii) of the fifth and sixth aspect is at least 1.2. In certain embodiments the factor in step (ii) of the fifth and sixth aspect is at least 1.3.

In certain embodiments the factor in step (iii) of the fifth and sixth aspect is at least 1.2. In certain embodiments the factor in step (iii) of the fifth and sixth aspect is at least 1.3.

The following paragraphs relate to all aspects of the present invention.

In certain embodiments the patient is a hypoparathyroidism patient who has had hypoparathyroidism for less than 5 years. In certain embodiments the patient is a hypoparathyroidism patient who has had hypoparathyroidism for between 5 and 10 years. In certain embodiments the patient is a hypoparathyroidism patient who has had hypoparathyroidism for more than 10 years.

In certain embodiments the long-acting PTH compound is administered once daily. In certain embodiments the long-acting PTH compound is administered once a week.

In certain embodiments the long-acting PTH compound is administered by injection. In certain embodiments the long-acting PTH compound is administered by subcutaneous injection. In certain embodiments the long-acting PTH compound is administered once daily by subcutaneous injection. In certain embodiments the long-acting PTH compound is administered once a week by subcutaneous injection.

Administration of the long-acting PTH compound leads to a normalization of bone turnover in patients having an increased BMD. In certain embodiments the reduction in BMD is associated with an initial increase in bone turnover markers that trends towards age- and sex-appropriate norms with prolonged use of the long-acting PTH compound. In certain embodiments the term “prolonged use” refers to a use of at least 58 weeks. In certain embodiments such bone turnover markers are P1NP and/or CTx. In certain embodiments administration of the long-acting PTH compound leads to a normalization of bone turnover markers by week 58. In certain embodiments mean values for both the anabolic marker P1NP and resorptive marker CTx are within the age- and sex-appropriate norms after 58 weeks of treatment, reflecting a normalization of bone turnover.

Treatment of patients with increased BMD results in a stabilization of BMD Z-scores.

In certain embodiments the long-acting PTH compound is a sustained-release PTH compound.

In certain embodiments the long-acting PTH compound is a compound of formula (Ta) or (Ib) or a pharmaceutically acceptable salt thereof

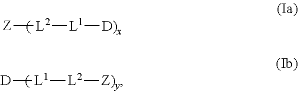

wherein
each -D is individually a PTH moiety;
each -L¹- is individually a linker moiety covalently and reversibly connected to -D;
each -L²- is individually a single chemical bond or a spacer moiety;
each —Z is individually a carrier moiety, such as a fatty acid derivative or a polymer;
x is an integer selected from the group consisting of 1, 2, 3, 4, 5, 6, 7, 8, 9, 10, 11, 12, 13, 14, 15, 16, 17, 18, 19, 20, 21, 22, 23, 24 and 25; and
y is an integer selected from the group consisting of 2, 3, 4 and 5.

The compounds of formula (Ia) and (Ib) are sustained-release PTH compounds.

In certain embodiments y of formula (Ib) is 2. In certain embodiments y of formula (Ib) is 3. In certain embodiments y of formula (Ib) is 4. In certain embodiments y of formula (Ib) is 5. In certain embodiments y of formula (Ib) is 6. In certain embodiments y of formula (Ib) is 7. In certain embodiments y of formula (Ib) is 8. In certain embodiments y of formula (Ib) is 9. In certain embodiments y of formula (Ib) is 10. In certain embodiments y of formula (Ib) is 11. In certain embodiments y of formula (Ib) is 12. In certain embodiments y of formula (Ib) is 13. In certain embodiments y of formula (Ib) is 14. In certain embodiments y of formula (Ib) is 15. In certain embodiments y of formula (Ib) is 16. In certain embodiments y of formula (Ib) is 17. In certain embodiments y of formula (Ib) is 18. In certain embodiments y of formula (Ib) is 19 In certain embodiments y of formula (Ib) is 20.

In certain embodiments the long-acting PTH compound is a compound of formula (Ia). In certain embodiments the long-acting PTH compound is a compound of formula (Ia) with x=1.

Specific embodiments for -D, -L¹-, -L²- and Z are as described elsewhere herein.

In certain embodiments the long-acting PTH compound is a conjugate or its pharmaceutically acceptable salt comprising a carrier moiety Z′ to which one or more moieties -L²-L¹-D are conjugated, wherein each -L²- is individually a chemical bond or a spacer moiety; each -L¹- is individually a linker moiety to which -D is reversibly and covalently conjugated; each -D is individually a PTH moiety; and Z′ is a hydrogel. Such long-acting PTH compound is a sustained-release PTH compound. Specific embodiments for -D, -L¹-, -L²- and Z′ are as described elsewhere herein.

In certain embodiments -D is a C-terminally truncated PTH, such as a C-terminally truncated PTH from human, such as a C-terminally truncated PTH selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120 and SEQ ID NO:121; and sequences having at least 90% homology thereto.

In certain embodiments -D is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120 and SEQ ID NO:121.

In certain embodiments -D is selected from the group consisting of SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120; and sequences having at least 90% homology thereto.

In certain embodiments -D is selected from the group consisting of SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119 and SEQ ID NO:120.

In certain embodiments -D is selected from the group consisting of SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115; and sequences having at least 90% homology thereto.

In certain embodiments -D is selected from the group consisting of SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:107, SEQ ID NO:108, SEQ ID NO:109, SEQ ID NO:110, SEQ ID NO:111, SEQ ID NO:112, SEQ ID NO:113, SEQ ID NO:114 and SEQ ID NO:115.

In certain embodiments -D is selected from the group consisting of SEQ ID NO:50, SEQ ID NO:51, SEQ ID NO:52, SEQ ID NO:110, SEQ ID NO:111, SEQ ID NO:112; and sequences having at least 90% homology thereto.

In certain embodiments -D is selected from the group consisting of SEQ ID NO:50, SEQ ID NO:51, SEQ ID NO:52, SEQ ID NO:110, SEQ ID NO:111 and SEQ ID NO:112.

In certain embodiments -D is of SEQ ID NO:50. In certain embodiments -D is of SEQ ID NO:52. In certain embodiments -D is of SEQ ID NO:110. In certain embodiments -D is of SEQ ID NO:111. In certain embodiments -D is of SEQ ID NO:112.

In certain embodiments -D is of SEQ ID NO:51.

In certain embodiments -D is of SEQ ID NO: 122:

AVAEIQLMHQRAKWIQDARRRAFLHKLIAEIHTAEI

A moiety -L¹- is either conjugated to a functional group of the side chain of an amino acid residue of -D, to the N-terminal amine functional group or to the C-terminal carboxyl functional group of -D or to a nitrogen atom in the backbone polypeptide chain of -D. Attachment to either the N-terminus or C-terminus can either be directly through the corresponding amine or carboxyl functional group, respectively, or indirectly, wherein a spacer moiety is first conjugated to the amine or carboxyl functional group to which spacer moiety -L¹- is conjugated.

In certain embodiments the amino acid residue of PTH to which -L¹- is conjugated comprises a functional group selected from the group consisting carboxylic acid, primary amine, secondary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, sulfate, disulfide, vinyl sulfone, vinyl ketone, diazoalkane, oxirane, guanidine and aziridine. In certain embodiments the amino acid residue of PTH to which -L¹- is conjugated comprises a functional group selected from the group consisting of hydroxyl, primary amine, secondary amine and guanidine. In certain embodiments the amino acid residue of PTH to which -L¹- is conjugated comprises a primary or secondary amine functional group. In certain embodiments the amino acid residue of PTH to which -L¹- is conjugated comprises a primary amine functional group.

If the moiety -L¹- is conjugated to a functional group of the side chain of an amino acid residue of PTH said amino acid residue is selected from the group consisting of proteinogenic amino acid residues and non-proteinogenic amino acid residues. In certain embodiments -L¹- is conjugated to a functional group of the side chain of a proteinogenic amino acid residue of PTH. In certain embodiments -L¹- is conjugated to a functional group of the side chain of a non-proteinogenic amino acid residue of PTH.

In certain embodiments -L¹- is conjugated to a functional group of the side chain of a proteinogenic amino acid residue of PTH. In certain embodiments said proteinogenic amino acid is selected from the group consisting of histidine, lysine, tryptophan, serine, threonine, tyrosine, aspartic acid, glutamic acid and arginine. In certain embodiments said proteinogenic amino acid is selected from the group consisting of lysine, aspartic acid, arginine and serine. In certain embodiments said proteinogenic amino acid is selected from the group consisting of lysine, arginine and serine. In certain embodiments -L¹- is conjugated to a functional group of the side chain of a histidine of PTH. In certain embodiments -L¹- is conjugated to a functional group of the side chain of a lysine of PTH. In certain embodiments -L¹- is conjugated to a functional group of the side chain of a tryptophan of PTH. In certain embodiments -L¹- is conjugated to a functional group of the side chain of a serine of PTH. In certain embodiments -L¹- is conjugated to a functional group of the side chain of a threonine of PTH. In certain embodiments -L¹- is conjugated to a functional group of the side chain of a tyrosine of PTH. In certain embodiments -L¹- is conjugated to a functional group of the side chain of an aspartic acid of PTH. In certain embodiments -L¹- is conjugated to a functional group of the side chain of a glutamic acid of PTH. In certain embodiments -L¹- is conjugated to a functional group of the side chain of an arginine of PTH. It is understood that not every PTH moiety may comprise all of these amino acid residues.

In certain embodiments -L¹- is conjugated to the N-terminal amine functional group of PTH, either directly through the corresponding amine functional group or indirectly wherein a spacer moiety is first conjugated to the amine functional group to which spacer moiety -L¹- is conjugated. In certain embodiments -L¹- is directly conjugated to the N-terminal amine functional group of PTH. In certain embodiments -L¹- is conjugated to the C-terminal functional group of PTH, either directly through the corresponding carboxyl functional group or indirectly wherein a spacer moiety is first conjugated to the carboxyl functional group to which spacer moiety -L¹- is conjugated. In certain embodiments -L¹- is directly conjugated to the N-terminal amine functional group of PTH.

The moiety -L¹- can be connected to -D through any type of linkage, provided that it is reversible. In certain embodiments -L¹- is connected to -D through a linkage selected from the group consisting of amide, ester, carbamate, acetal, aminal, imine, oxime, hydrazone, disulfide and acylguanidine. In certain embodiments -L¹- is connected to -D through a linkage selected from the group consisting of amide, ester, carbamate and acylguanidin. It is understood that some of these linkages are not reversible per se, but that in the present invention neighboring groups comprised in -L¹- render these linkages reversible. In certain embodiments -L¹- is connected to -D through an ester linkage. In certain embodiments -L¹- is connected to -D through a carbamate linkage. In certain embodiments -L¹- is connected to -D through an acylguanidine. In certain embodiments -L¹- is connected to -D through an amide linkage.

The moiety -L¹- is a reversible prodrug linker from which the drug, i.e. PTH, is released in its free form, i.e. it is a traceless prodrug linker. Suitable prodrug linkers are known in the art, such as for example the reversible prodrug linker moieties disclosed in WO 2005/099768 A2, WO 2006/136586 A2, WO 2011/089216 A1 and WO 2013/024053 A1, which are incorporated by reference herewith.

In certain embodiments -L¹- is a reversible prodrug linker as described in WO 2011/012722 A1, WO 2011/089214 A1, WO 2011/089215 A1, WO 2013/024052 A1 and WO 2013/160340 A1 which are incorporated by reference herewith.

In certain embodiments -L¹- is disclosed in WO 2009/095479 A2. Accordingly, in certain embodiments the moiety -L¹- is of formula (II):

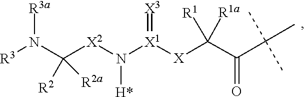

wherein the dashed line indicates attachment to a nitrogen, hydroxyl or thiol of -D;
—X— is selected from the group consisting of —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—;
C(R⁴R^{4a})—O—; —O—C(R⁴R^{4a})—; and —C(R⁷R^{7a})—;
X¹ is selected from the group consisting of C; and S(O);
—X²— is selected from the group consisting of —C(R⁸R^{8a})—; and —C(R⁸R^{8a})—C(R⁹R^{9a})—;
═X³ is selected from the group consisting of ═O; ═S; and ═N—CN;
—R¹, —R^{1a}, —R², —R^{2a}, —R⁴, —R^{4a}, —R⁵, —R^{5a}, —R⁶, —R⁸, —R^{8a}, —R⁹, and —R^{9a} are independently selected from the group consisting of —H; and C₁₋₆ alkyl;
—R³, and —R^{3a} are independently selected from the group consisting of —H; and C₁₋₆ alkyl, provided that in case one of —R³, —R^{3a} or both are other than —H they are connected to N to which they are attached through an SP³-hybridized carbon atom;
—R⁷ is selected from the group consisting of —N(R¹⁰R^{10a}); and —NR¹⁰—(C═O)—R¹¹;
—R^{7a}, —R¹⁰, —R¹⁰, and —R¹¹ are independently of each other selected from the group consisting of —H; and C₁₋₆ alkyl;
optionally, one or more of the pairs —R^{1a}/—R^{4a}, —R^{1a}/—R^{5a}, —R^{1a}/—R^{7a}, —R^{4a}/—R^{5a}, and —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R⁸/—R^{8a}, and —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹, and —R²/—R³ are joined together with the atoms to which they are attached to form a ring A;
optionally, R³/R^{3a} are joined together with the nitrogen atom to which they are attached to form a 3- to 10-membered heterocycle;
A is selected from the group consisting of phenyl; naphthyl; indenyl; indanyl; tetralinyl;
C₃₋₁₀ cycloalkyl; 3- to 10-membered heterocyclyl; and 8- to 11-membered heterobicyclyl; and
wherein -L¹- is substituted with at least one -L²-Z or -L²-Z′ and wherein -L¹- is optionally further substituted, provided that the hydrogen marked with the asterisk in formula (II) is not replaced by -L²-Z or -L²-Z′ or a substituent.

In certain embodiments -L¹- of formula (II) is substituted with one moiety -L²-Z or -L²-Z′.

In certain embodiments -L¹- of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles may be formed in which the atoms directly attached to the nitrogen are SP³-hybridized carbon atoms. In other words, such 3- to 10-membered heterocycle formed by -R³/—R^{3a} together with the nitrogen atom to which they are attached has the following structure:

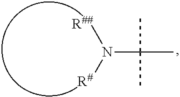

wherein
the dashed line indicates attachment to the rest of -L¹-;
the ring comprises 3 to 10 atoms comprising at least one nitrogen; and
R^{#} and R^{##} represent an sp³-hybridized carbon atom.

It is also understood that the 3- to 10-membered heterocycle may be further substituted.

Exemplary embodiments of suitable 3- to 10-membered heterocycles formed by -R³/—R^{3a} of formula (II) together with the nitrogen atom to which they are attached are the following:

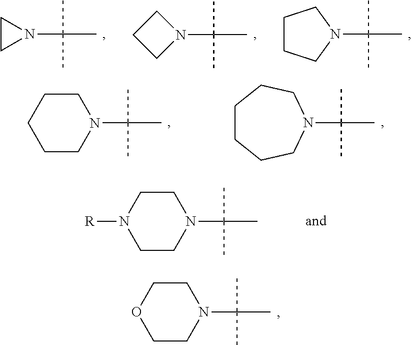

wherein
dashed lines indicate attachment to the rest of the molecule; and
—R is selected from the group consisting of —H and C₁₋₆ alkyl.

-L¹- of formula (II) may optionally be further substituted. In general, any substituent may be used as far as the cleavage principle is not affected, i.e. the hydrogen marked with the asterisk in formula (II) is not replaced and the nitrogen of the moiety

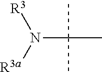

of formula (II) remains part of a primary, secondary or tertiary amine, i.e. —R³ and —R^{3a} are independently of each other —H or are connected to —N< through an sp³-hybridized carbon atom.

In one embodiment —R¹ or —R^{1a} of formula (II) is substituted with -L²-Z or -L²-Z′. In another embodiment —R² or —R^{2a} of formula (II) is substituted with -L²-Z or -L²-Z′. In another embodiment —R³ or —R^{3a} of formula (II) is substituted with -L²-Z or -L²-Z′. In another embodiment —R⁴ of formula (II) is substituted with -L²-Z or -L²-Z′. In another embodiment —R⁵ or —R^{5a} of formula (II) is substituted with -L²-Z or -L²-Z′. In another embodiment —R⁶ of formula (II) is substituted with -L²-Z or -L²-Z′. In another embodiment —R or —R^{7a} of formula (II) is substituted with -L²-Z or -L²-Z′. In another embodiment —R⁸ or —R^{8a} of formula (II) is substituted with -L²-Z or -L²-Z′. In another embodiment —R⁹ or —R^{9a} of formula (II) is substituted with -L²-Z or -L²-Z′. In another embodiment —R¹⁰ is substituted with -L²-Z or -L²-Z′. In another embodiment —R¹¹ is substituted with -L²-Z or -L²-Z′. In certain embodiments —R of formula (II) is substituted with -L²-Z or -L²-Z′.

In certain embodiments —X-of formula (II) is selected from the group consisting of —C(R⁴R^{4a})—, —N(R⁴)— and —C(R⁷R^{7a})—. In certain embodiments —X-of formula (II) is —C(R⁴R^{4a})—. In certain embodiments —X-of formula (II) is —C(R⁷R^{7a})—.

In certain embodiments —R⁷ of formula (II) is —NR¹⁰—(C═O)—R¹¹.

In certain embodiments —R^{7a} of formula (II) is selected from —H, methyl and ethyl. In certain embodiments —R^{7a} of formula (II) is —H.

In certain embodiments —R¹⁰ is selected from —H, methyl and ethyl. In certain embodiments —R¹⁰ is methyl.

In certain embodiments —R¹ is selected from —H, methyl and ethyl. In certain embodiments —R¹¹ is —H. In certain embodiments —R¹¹ is substituted with -L²-Z or -L²-Z′.

In certain embodiments —X— of formula (II) is —N(R⁴)—.

In certain embodiments —R⁴ is selected from the group consisting of —H, methyl and ethyl. In certain embodiments —R⁴ is —H.

In certain embodiments X¹ of formula (II) is C.

In certain embodiments ═X³ of formula (II) is ═O.

In certain embodiments —X²— of formula (II) is —C(R⁸R^{8a})—.

In certain embodiments —R⁸ and —R^{8a} of formula (II) are independently selected from the group consisting of —H, methyl and ethyl. In certain embodiments at least one of —R⁸ and —R^{8a} of formula (II) is —H. In certain embodiments both —R⁸ and —R^{8a} of formula (II) are —H.

In certain embodiments —R¹ and —R^{1a} of formula (II) are independently selected from the group consisting of —H, methyl and ethyl.

In certain embodiments at least one of —R¹ and —R^{1a} of formula (II) is —H. In certain embodiments —R¹ and —R^{1a} of formula (II) are —H.

In certain embodiments at least one of —R¹ and —R^{1a} of formula (II) is methyl. In certain embodiments both —R¹ and —R^{1a} of formula (II) are methyl.

In certain embodiments —R² and —R^{2a} of formula (II) are independently selected from the group consisting of —H, methyl and ethyl. In certain embodiments at least one of —R² and —R^{2a} of formula (II) is —H. In certain embodiments both —R² and —R^{2a} of formula (II) are H.

In certain embodiments —R³ and —R^{3a} of formula (II) are independently selected from the group consisting of —H, methyl, ethyl, propyl and butyl.

In certain embodiments at least one of —R³ and —R^{3a} of formula (II) is methyl. In certain embodiments —R³ of formula (II) is methyl and —R^{3a} of formula (II) is —H.

In certain embodiments —R³ and —R^{3a} of formula (II) are both —H.

In certain embodiments -D is connected to -L¹- through a nitrogen by forming an amide bond.

In certain embodiments the moiety -L¹- is of formula (IIa-i):

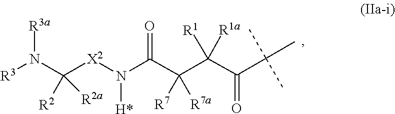

wherein
the dashed line indicates attachment to a nitrogen of -D through an amide bond;
—R¹, —R^{1a}, —R², —R^{2a}, —R³, —R^{3a}, —R⁷, —R^{7a} and —X²— are used as defined in formula (II); and
wherein -L¹- is substituted with at least one -L²-Z or -L²-Z′ and wherein -L¹- is optionally further substituted, provided that the hydrogen marked with the asterisk in formula (IIa-i) is not replaced by -L²-Z or -L²-Z′ or a substituent.

It is understood that in case one of —R³, —R^{3a} of formula (IIa-i) or both are other than —H they are connected to N to which they are attached through an sp³-hybridized carbon atom.

In certain embodiments -L¹- of formula (IIa-i) is substituted with one moiety -L²-Z or -L²-Z′.

In certain embodiments the moiety -L¹- of formula (IIa-i) is not further substituted.

In certain embodiments —R¹ and —R^{1a} of formula (IIa-i) are independently selected from the group consisting of —H, methyl and ethyl. In certain embodiments at least one of —R¹ and —R^{1a} of formula (IIa-i) is —H. In certain embodiments both —R¹ and —R^{1a} of formula (IIa-i) are —H.

In certain embodiments —R⁷ of formula (IIa-i) is —NR¹⁰—(C═O)—R¹¹.

In certain embodiments —R^{7a} of formula (II-i) is selected from —H, methyl and ethyl. In certain embodiments —R^{7a} of formula (II-i) is —H.

In certain embodiments —R¹⁰ of formula (IIa-i) is selected from —H, methyl and ethyl. In certain embodiments —R¹⁰ of formula (IIa-i) is methyl.

In certain embodiments —R¹¹ of formula (IIa-i) is selected from —H, methyl and ethyl. In certain embodiments —R¹¹ of formula (IIa-i) is —H.

In certain embodiments —R¹¹ of formula (IIa-i) is substituted with -L²-Z or -L²-Z′.

In certain embodiments —X²— of formula (IIa-i) is —C(R⁸R^{8a})—.

In certain embodiments —R⁸ and —R^{8a} of formula (IIa-i) are independently selected from the group consisting of —H, methyl and ethyl. In certain embodiments at least one of —R⁸ and —R^{8a} of formula (IIa-i) is —H. In certain embodiments both —R⁸ and —R^{8a} of formula (IIa-i) are —H.

In certain embodiments R² and —R^{2a} of formula (IIa-i) are independently selected from the group consisting of —H, methyl and ethyl. In certain embodiments at least one of —R² and —R^{2a} of formula (IIa-i) is —H. In certain embodiments both —R² and —R^{2a} of formula (IIa-i) are H.

In certain embodiments —R³ and —R^{3a} of formula (IIa-i) are independently selected from the group consisting of —H, methyl, ethyl, propyl and butyl. In certain embodiments at least one of —R³ and —R^{3a} of formula (IIa-i) is methyl.

In certain embodiments —R³ of formula (IIa-i) is —H and —R^{3a} of formula (IIa-i) is methyl.

In certain embodiments the moiety -L¹- is of formula (IIa-ii):

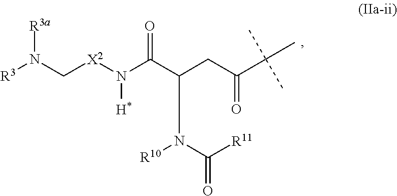

wherein the dashed line indicates attachment to a nitrogen of -D through an amide bond;
—R², —R^{2a}, —R¹⁰, —R¹¹ and —X²— are used as defined in formula (II); and
wherein -L¹- is substituted with at least one -L²-Z or -L²-Z′ and wherein -L¹- is optionally further substituted, provided that the hydrogen marked with the asterisk in formula (IIa-ii) is not replaced by -L²-Z or -L²-Z′ or a substituent.

It is understood that in case one of —R³, —R^{3a} of formula (IIa-ii) or both are other than —H they are connected to N to which they are attached through an sp³-hybridized carbon atom.

In certain embodiments -L¹- of formula (IIa-ii) is substituted with one moiety -L²-Z or -L²-Z′.

In certain embodiments the moiety -L¹- of formula (IIa-ii) is not further substituted.

In certain embodiments —X²— of formula (IIa-ii) is —C(R⁸R^{8a})—.

In certain embodiments —R⁸ and —R^{8a} of formula (IIa-ii) are independently selected from the group consisting of —H, methyl and ethyl. In certain embodiments at least one of —R⁸ and —R^{8a} of formula (IIa-ii) is —H. In certain embodiments both —R⁸ and —R^{8a} of formula (IIa-ii) are —H.

In certain embodiments —R³ and —R^{3a} of formula (IIa-ii) are independently selected from the group consisting of —H, methyl, ethyl, propyl and butyl. In certain embodiments at least one of —R³ and —R^{3a} of formula (IIa-ii) is methyl.

In certain embodiments —R³ of formula (IIa-ii) is —H and —R^{3a} of formula (IIa-ii) is methyl.

In certain embodiments —R¹⁰ of formula (IIa-ii) is selected from —H, methyl and ethyl. In certain embodiments —R¹⁰ of formula (IIa-ii) is methyl.

In certain embodiments —R¹ of formula (IIa-ii) is selected from —H, methyl and ethyl. In certain embodiments —R¹ of formula (IIa-ii) is —H.

In certain embodiments —R¹¹ of formula (IIa-ii) is substituted with -L²-Z or -L²-Z′.

In certain embodiments the moiety -L¹- is of formula (IIa-ii′):

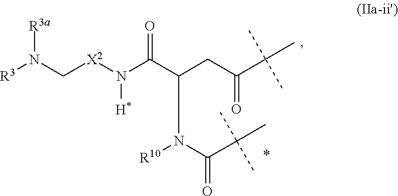

wherein
wherein the dashed line indicates attachment to a nitrogen of -D through an amide bond;
the dashed line marked with the asterisk indicates attachment to -L²-;
—R³, a —R¹⁰ and —X²— are used as defined in formula (II); and
wherein -L¹- is optionally further substituted, provided that the hydrogen marked with the asterisk in formula (IIa-ii′) is not replaced by a substituent.

It is understood that in case one of —R³, —R^{3a} of formula (IIa-ii′) or both are other than —H they are connected to N to which they are attached through a sp³-hybridized carbon atom.

In certain embodiments —X²— of formula (IIa-ii′) is —C(R⁸R^{8a})—.

In certain embodiments —R⁸ and —R^{8a} of formula (IIa-ii′) are independently selected from the group consisting of —H, methyl and ethyl. In certain embodiments at least one of —R⁸ and —R^{8a} of formula (IIa-ii′) is —H. In certain embodiments both —R⁸ and —R^{8a} of formula (IIa-ii′) are —H.

In certain embodiments —R³ and —R^{3a} of formula (IIa-ii′) are independently selected from the group consisting of —H, methyl, ethyl, propyl and butyl. In certain embodiments at least one of —R³ and —R^{3a} of formula (IIa-ii′) is methyl.

In certain embodiments —R³ of formula (IIa-ii′) is —H and —R^{3a} of formula (IIa-ii′) is methyl.

In certain embodiments —R¹⁰ of formula (IIa-ii′) is selected from —H, methyl and ethyl. In certain embodiments —R¹⁰ of formula (IIa-ii′) is methyl.

In certain embodiments the moiety -L¹- is of formula (IIa-iii):

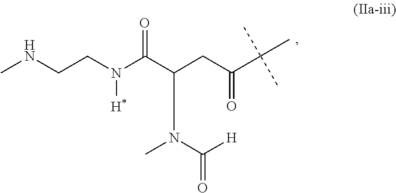

wherein the dashed line indicates attachment to a nitrogen of through an amide bond;
and wherein -L¹- is substituted with -L²-Z or -L²-Z′ and wherein -L¹- is optionally further substituted, provided that the hydrogen marked with the asterisk in formula (IIa-iii) is not replaced by -L²-Z or -L²-Z′ or a substituent.

It is understood that in case one of —R³, —R^{3a} of formula (IIa-iii) or both are other than —H they are connected to N to which they are attached through an sp³-hybridized carbon atom.

In certain embodiments the moiety -L¹- of formula (IIa-iii) is not further substituted.

In certain embodiments the moiety -L¹- is of formula (IIa-iii′):

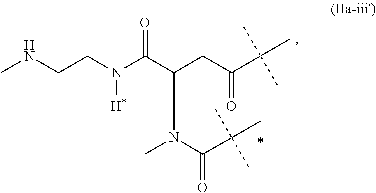

wherein
wherein the dashed line indicates attachment to a nitrogen of -D through an amide bond;
the dashed line marked with the asterisk indicates attachment to -L²-;
R², —R^{2a}, —R³, —R^{3a} and —X²— are used as defined in formula (II); and
wherein -L¹- is optionally further substituted, provided that the hydrogen marked with the asterisk in formula (IIa-iii′) is not replaced by a substituent.

It is understood that in case one of —R³, —R^{3a} of formula (IIa-iii′) or both are other than —H they are connected to N to which they are attached through an sp³-hybridized carbon atom.

In certain embodiments the moiety -L¹- of formula (IIa-iii′) is not further substituted.

In certain embodiments the moiety -L¹- is of formula (IIb-i)

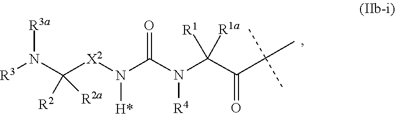

wherein
the dashed line indicates attachment to a nitrogen of -D through an amide bond;
—R¹, —R^{1a}, —R², —R^{2a}, —R³, —R^{3a}, —R⁴ and —X²— are used as defined in formula (II); and
wherein -L¹- is substituted with at least one -L²-Z or -L²-Z′ and wherein -L¹- is optionally further substituted, provided that the hydrogen marked with the asterisk in formula (IIb-i) is not replaced by -L²-Z or -L²-Z′ or a substituent.

It is understood that in case one of —R³, —R^{3a} of formula (IIb-i) or both are other than —H they are connected to N to which they are attached through an sp³-hybridized carbon atom.

In certain embodiments -L¹- of formula (IIb-i) is substituted with one moiety -L²-Z or -L²-Z′.

In certain embodiments the moiety -L¹- of formula (IIb-i) is not further substituted.

In certain embodiments —R¹ and —R^{1a} of formula (IIb-i) are independently selected from the group consisting of —H, methyl and ethyl. In certain embodiments at least one of —R¹ and —R^{1a} of formula (IIb-i) is methyl. In certain embodiments both —R¹ and —R^{1a} of formula (IIb-i) are methyl.

In certain embodiments —R⁴ of formula (IIb-i) is selected from the group consisting of —H, methyl and ethyl. In certain embodiments —R⁴ of formula (IIb-i) is —H.

In certain embodiments —X²— of formula (IIb-i) is —C(R⁸R^{8a})—.

In certain embodiments —R⁸ and —R^{8a} of formula (IIb-i) are independently selected from the group consisting of —H, methyl and ethyl. In certain embodiments at least one of —R⁸ and —R^{8a} of formula (IIb-i) is —H. In certain embodiments both —R⁸ and —R^{8a} of formula (IIb-i) are —H.

In certain embodiments —R² and —R^{2a} of formula (IIb-i) are independently selected from the group consisting of —H, methyl and ethyl. In certain embodiments at least one of —R² and —R^{2a} of formula (IIb-i) is —H. In certain embodiments both —R² and —R^{2a} of formula (IIb-i) are H.

In certain embodiments —R³ and —R^{3a} of formula (IIb-i) are independently selected from the group consisting of —H, methyl, ethyl, propyl and butyl. In certain embodiments at least one of —R³ and —R^{3a} of formula (IIb-i) is —H. In certain embodiments both —R³ and —R^{3a} of formula (IIb-i) are —H.

In certain embodiments the moiety -L¹- is of formula (IIb-ii):

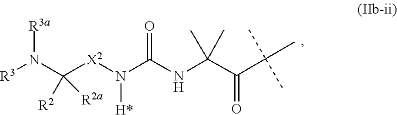

wherein the dashed line indicates attachment to a nitrogen of -D through an amide bond:
—R², —R^{2a}, —R³, —R^{3a} and —X²— are used as defined in formula (II); and
wherein -L¹- is substituted with at least one -L²-Z or -L²-Z′ and wherein -L¹- is optionally further substituted, provided that the hydrogen marked with the asterisk in formula (IIb-ii) is not replaced by -L²-Z or -L²-Z′ or a substituent.

It is understood that in case one of —R³, —R^{3a} of formula (IIb-ii) or both are other than —H they are connected to N to which they are attached through an sp³-hybridized carbon atom.

In certain embodiments -L¹- of formula (IIb-ii) is substituted with one moiety -L²-Z or -L²-Z′.

In certain embodiments the moiety -L¹- of formula (IIb-ii) is not further substituted.

In certain embodiments —X²— of formula (IIb-ii) is —C(R⁸R^{8a})—.

In certain embodiments —R⁸ and —R^{8a} of formula (IIb-ii) are independently selected from the group consisting of —H, methyl and ethyl. In certain embodiments at least one of —R⁸ and —R^{8a} of formula (IIb-ii) is —H. In certain embodiments both —R⁸ and —R^{8a} of formula (IIb-ii) are —H.

In certain embodiments —R² and —R^{2a} of formula (IIb-ii) are independently selected from the group consisting of —H, methyl and ethyl. In certain embodiments at least one of —R² and —R^{2a} of formula (IIb-ii) is —H. In certain embodiments both —R² and —R^{2a} of formula (IIb-ii) are H.

In certain embodiments —R³ and —R^{3a} of formula (IIb-ii) are independently selected from the group consisting of —H, methyl, ethyl, propyl and butyl. In certain embodiments at least one of —R³ and —R^{3a} of formula (IIb-ii) is —H. In certain embodiments both —R³ and —R^{3a} of formula (IIb-ii) are —H.

In certain embodiments the moiety -L¹- is of formula (IIb-ii′):

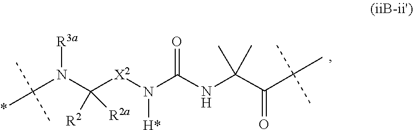

wherein
the dashed line indicates attachment to a nitrogen of -D through an amide bond;
the dashed line marked with the asterisk indicates attachment to -L²-;
R², —R^{2a}, —R^{3a} and —X²— are used as defined in formula (II); and
wherein -L¹- is optionally further substituted, provided that the hydrogen marked with the asterisk in formula (IIb-ii′) is not replaced by a substituent.

It is understood that in case —R^{3a} of formula (IIb-ii′) is other than —H it is connected to N to which it is attached through an sp³-hybridized carbon atom.

In certain embodiments the moiety -L¹- of formula (IIb-ii′) is not further substituted.

In certain embodiments —X²— of formula (IIb-ii′) is —C(R⁸R^{8a})—.

In certain embodiments —R⁸ and —R^{8a} of formula (IIb-ii′) are independently selected from the group consisting of —H, methyl and ethyl. In certain embodiments at least one of —R⁸ and —R^{8a} of formula (IIb-ii′) is —H. In certain embodiments both —R⁸ and —R^{8a} of formula (IIb-ii′) are —H.

In certain embodiments —R² and —R^{2a} of formula (IIb-ii′) are independently selected from the group consisting of —H, methyl and ethyl. In certain embodiments at least one of —R² and —R^{2a} of formula (IIb-ii′) is —H. In certain embodiments both —R² and —R^{2a} of formula (IIb-ii′) are H.

In certain embodiments —R^{3a} of formula (IIb-ii′) is selected from the group consisting of —H, methyl, ethyl, propyl and butyl. In one embodiment —R^{3a} of formula (IIb-ii′) is —H.

In certain embodiments the moiety -L¹- is of formula (IIb-iii):

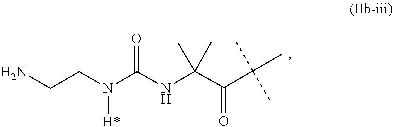

wherein
the dashed line indicates attachment to a nitrogen of -D through an amide bond; and
wherein -L¹- is substituted with at least one -L²-Z or -L²-Z′ and wherein -L¹- is optionally further substituted, provided that the hydrogen marked with the asterisk in formula (IIb-iii) is not replaced by -L²-Z or -L²-Z′ or a substituent.

It is understood that in case one of —R³, —R^{3a} of formula (IIb-iii) or both are other than —H they are connected to N to which they are attached through an sp³-hybridized carbon atom.

In certain embodiments -L¹- of formula (IIb-iii) is substituted with one moiety -L²-Z or -L²-Z′.

In certain embodiments the moiety -L¹- of formula (IIb-iii) is not further substituted.

In certain embodiments the moiety -L¹- is of formula (IIb-iii′):

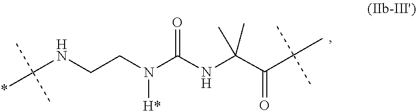

wherein
the dashed line indicates attachment to a nitrogen of -D through an amide bond;
the dashed line marked with the asterisk indicates attachment to -L²-; and
wherein -L¹- is optionally further substituted, provided that the hydrogen marked with the asterisk in formula (IIb-iii′) is not replaced by a substituent.

It is understood that the nitrogen adjacent to the dashed line marked with the asterisk in formula (IIb-iii′) is attached to -L²- through an sp³-hybridized carbon atom.

In certain embodiments the moiety -L¹- of formula (IIb-iii′) is not further substituted.

In certain embodiments -L¹- is disclosed in WO2016/020373A1. Accordingly, in certain embodiments the moiety -L¹- is of formula (III):

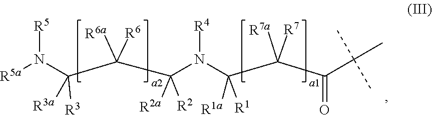

wherein
the dashed line indicates attachment to a primary or secondary amine or hydroxyl of -D through an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R¹, —C≡N, —C(═NR⁸)R^{8a}, —CR⁸(═CR^{8a}R^{8b}), —C≡CR⁸ and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, and —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O) N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R^{1a})C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, and —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R¹³, and —R^{13b} is independently selected from the group consisting of —H, and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁶/—R^{6a}, and —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, and —R⁶/—R⁷ are joint together with the atoms to which they are attached to form a ring A;
A is selected from the group consisting of phenyl; naphthyl; indenyl; indanyl; tetralinyl;
C₃₋₁₀ cycloalkyl; 3- to 10-membered heterocyclyl; and 8- to 11-membered heterobicyclyl;
wherein -L¹- is substituted with at least one -L²-Z or -L²-Z′ and wherein -L¹- is optionally further substituted.

The optional further substituents of -L¹- of formula (III) are in certain embodiments as described above.

In certain embodiments -L¹- of formula (III) is substituted with one moiety -L²-Z or -L²-Z′.

In certain embodiments -L¹- of formula (III) is not further substituted.

In certain embodiments -L¹- is as disclosed in EP1536334B1, WO2009/009712A1, WO2008/034122A1, WO2009/143412A2, WO2011/082368A2, and U.S. Pat. No. 8,618,124B2, which are herewith incorporated by reference in their entirety.

In certain embodiments -L¹- is as disclosed in U.S. Pat. No. 8,946,405B2 and U.S. Pat. No. 8,754,190B2, which are herewith incorporated by reference in their entirety. Accordingly, in certain embodiments -L¹- is of formula (IV):

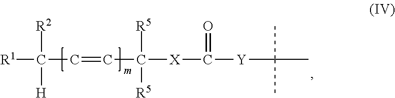

wherein
   the dashed line indicates attachment to -D and wherein attachment is through a functional group of -D selected from the group consisting of —OH, —SH and —NH₂;
   m is 0 or 1;
   at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴, one and only one of —R¹ and —R² is selected from the group consisting of —H, optionally substituted alkyl, optionally substituted arylalkyl, and optionally substituted heteroarylalkyl;
   —R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
   —R⁴ is selected from the group consisting of optionally substituted alkyl, optionally substituted aryl, optionally substituted arylalkyl, optionally substituted heteroaryl, and optionally substituted heteroarylalkyl;
   each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
   —R⁹ is selected from the group consisting of —H and optionally substituted alkyl;
   —Y— is absent and —X— is —O— or —S—; or
   —Y— is —N(Q)CH₂— and —X— is —O—;
   Q is selected from the group consisting of optionally substituted alkyl, optionally substituted aryl, optionally substituted arylalkyl, optionally substituted heteroaryl and optionally substituted heteroarylalkyl;
   optionally, —R¹ and —R² may be joined to form a 3 to 8-membered ring; and
   optionally, both —R⁹ together with the nitrogen to which they are attached form a heterocyclic ring;
   wherein -L¹- is substituted with at least one -L²-Z or -L²-Z′ and wherein -L¹- is optionally further substituted.

Only in the context of formula (IV) the terms used have the following meaning:

The term “alkyl” as used herein includes linear, branched or cyclic saturated hydrocarbon groups of 1 to 8 carbons, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, such as 6 to 10 carbons, including groups such as phenyl, naphthyl, and anthracenyl. The term “heteroaryl” includes aromatic rings comprising 3 to 15 carbons containing at least one N, O or S atom, such as 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted.

Optional substituents on any group, including the above, include halo, nitro, cyano, —OR, —SR, —NR₂, —OCOR, —NRCOR, —COOR, —CONR₂, —SOR, —SO₂R, —SONR₂, —SO₂NR₂, wherein each R is independently alkyl, alkenyl, alkynyl, aryl or heteroaryl, or two R groups taken together with the atoms to which they are attached form a ring.

In certain embodiments -L¹- of formula (IV) is substituted with one moiety -L²-Z or -L²-Z′.

In certain embodiments -L¹- of formula (IV) is not further substituted.

In certain embodiments -L¹- is as disclosed in WO2013/036857A1, which is herewith incorporated by reference in its entirety. Accordingly, in certain embodiments -L¹- is of formula (V):

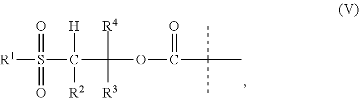

wherein
the dashed line indicates attachment to -D through an amine functional group of -D;
—R¹ is selected from the group consisting of optionally substituted C₁-C₆ linear, branched, or cyclic alkyl; optionally substituted aryl; optionally substituted heteroaryl;
alkoxy; and —NR⁵2;
—R² is selected from the group consisting of —H; optionally substituted C₁-C₆ alkyl;
optionally substituted aryl; and optionally substituted heteroaryl;
—R³ is selected from the group consisting of —H; optionally substituted C₁-C₆ alkyl;
optionally substituted aryl; and optionally substituted heteroaryl;
—R⁴ is selected from the group consisting of —H; optionally substituted C₁-C₆ alkyl;
optionally substituted aryl; and optionally substituted heteroaryl;
each —R⁵ is independently of each other selected from the group consisting of —H;
optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with at least one -L²-Z or -L²-Z′ and wherein -L¹- is optionally further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, such as 6-10 carbons, including groups such as phenyl, naphthyl, and anthracene “Heteroaryl” includes aromatic rings comprising 3-15 carbons containing at least one N, O or S atom, such as 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketne; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

In certain embodiments -L¹- of formula (V) is substituted with one moiety -L²-Z or -L²-Z′.

In certain embodiments -L¹- of formula (V) is not further substituted.

In certain embodiments -L¹- is as disclosed in U.S. Pat. No. 7,585,837B2, which is herewith incorporated by reference in its entirety. Accordingly, in certain embodiments -L¹- is of formula (VI):

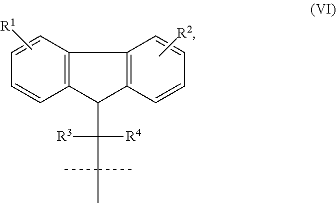

wherein
the dashed line indicates attachment to -D through an amine functional group of -D;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with at least one -L²-Z or -L²-Z′ and wherein -L¹- is optionally further substituted.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, such as 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments -L¹- of formula (VI) is substituted with one moiety -L²-Z or -L²-Z′.

In certain embodiments -L¹- of formula (VI) is not further substituted.

A further preferred embodiment for -L¹- is disclosed in WO2002/089789A1, which is herewith incorporated by reference in its entirety. Accordingly, a preferred moiety -L¹- is of formula (VII):

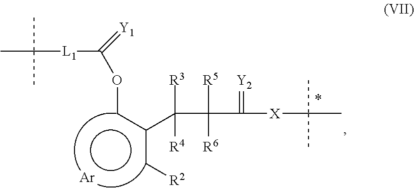

wherein
the dashed line indicates attachment to -D through an amine functional group of -D;
L₁ is a bifunctional linking group,
Y₁ and Y₂ are independently 0, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with at least one -L²-Z or -L²-Z′ and wherein -L¹- is optionally further substituted.

Only in the context of formula (VII) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moieities such as methoxy; and phenoxy includes moieties such as 3-nitrophenoxy. Halo-shall be understood to include fluoro, chloro, iodo and bromo.

In certain embodiments -L¹- of formula (VII) is substituted with one moiety -L²-Z or -L²-Z′.

In certain embodiments -L¹- of formula (VII) is not further substituted.

In certain embodiments -L¹- comprises a substructure of formula (VIII)

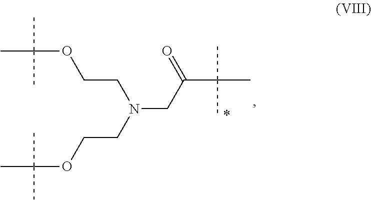

wherein
the dashed line marked with the asterisk indicates attachment to a nitrogen of -D through an amide bond;
the unmarked dashed lines indicate attachment to the remainder of -L¹-; and
wherein -L¹- is substituted with at least one -L²-Z or -L²-Z′ and wherein -L¹- is optionally further substituted.

In certain embodiments -L¹- of formula (VIII) is substituted with one moiety -L²-Z or -L²-Z′.

In certain embodiments -L¹- of formula (VIII) is not further substituted.

In certain embodiments -L¹- comprises a substructure of formula (IX)

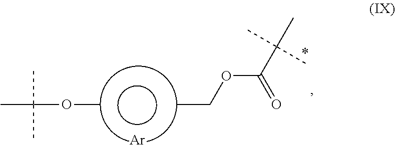

wherein
the dashed line marked with the asterisk indicates attachment to a nitrogen of -D through a carbamate bond;
the unmarked dashed lines indicate attachment to the remainder of -L¹-; and
wherein -L¹- is substituted with at least one -L²-Z or -L²-Z′ and wherein -L¹- is optionally further substituted.

In certain embodiments -L¹- of formula (IX) is substituted with one moiety -L²-Z or -L²-Z′.

In certain embodiments -L¹- of formula (IX) is not further substituted.

In certain embodiments -L¹- has a structure as disclosed in WO2020/206358 A1. Accordingly, in certain embodiments the moiety -L¹- is of formula (X):

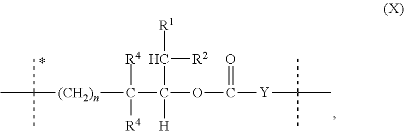

wherein
the unmarked dashed line indicates attachment to -D;
the dashed line marked with the asterisk indicates attachment to -L²-Z or -L²-Z′;
n is an integer selected from the group consisting of 0, 1, 2, 3, 4, 5 and 6;
—R¹ and —R² are independently an electron-withdrawing group, alkyl, or —H, and wherein at least one of —R¹ or —R² is an electron-withdrawing group;
each —R⁴ is independently C₁-C₃ alkyl or the two —R⁴ are taken together with the carbon atom to which they are attached to form a 3- to 6-membered ring; and
—Y— is absent when -D is a drug moiety connected through an amine, or —Y— is —N(R⁶)CH₂— when -D is a drug moiety connected through a phenol, alcohol, thiol, thiophenol, imidazole, or non-basic amine; wherein —R⁶ is optionally substituted C₁-C₆ alkyl, optionally substituted aryl, or optionally substituted heteroaryl.

In certain embodiments n of formula (X) is an integer selected from 1, 2, 3, 4, 5 and 6. In certain embodiments n of formula (X) is an integer selected from 1, 2 and 3. In certain embodiments n of formula (X) is an integer from 0, 1, 2 and 3. In certain embodiments n of formula (X) is 1. In certain embodiments n of formula (X) is 2. In certain embodiments n of formula (X) is 3.

In certain embodiments the electron-withdrawing group of —R¹ and —R² of formula (X) is selected from the group consisting of —CN; —NO₂; optionally substituted aryl; optionally substituted heteroaryl; optionally substituted alkenyl; optionally substituted alkynyl; —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments the electron-withdrawing group of —R¹ and —R² of formula (X) is —CN. In certain embodiments the electron-withdrawing group of —R¹ and —R² of formula (X) is —NO₂. In certain embodiments the electron-withdrawing group of —R¹ and —R² of formula (X) is optionally substituted aryl comprising 6 to 10 carbons. In certain embodiments the electron-withdrawing group of —R¹ and —R² of formula (X) is optionally substituted phenyl, naphthyl, or anthracenyl. In certain embodiments the electron-withdrawing group of —R¹ and —R² of formula (X) is optionally substituted heteroaryl comprising 3 to 7 carbons and comprising at least one N, O, or S atom. In certain embodiments the electron-withdrawing group of —R¹ and —R² of formula (X) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments the electron-withdrawing group of —R¹ and —R² of formula (X) is optionally substituted alkenyl containing 2 to 20 carbon atoms. In certain embodiments the electron-withdrawing group of —R¹ and —R² of formula (X) is optionally substituted alkynyl comprising 2 to 20 carbon atoms. In certain embodiments the electron-withdrawing group of —R¹ and —R² of formula (X) is —COR³, —SOR³, or —SO₂R³, wherein —R³ is —H, optionally substituted alkyl comprising 1 to 20 carbon atoms, optionally substituted aryl, optionally substituted arylalkyl, optionally substituted heteroaryl, optionally substituted heteroarylalkyl, —OR⁸ or —NR⁸², wherein each —R⁸ is independently —H or optionally substituted alkyl comprising 1 to 20 carbon atoms, or both —R⁸ groups are taken together with the nitrogen to which they are attached to form a heterocyclic ring. In certain embodiments the electron-withdrawing group of —R¹ and —R² of formula (X) is —SR⁹, wherein —R⁹ is optionally substituted alkyl comprising 1 to 20 carbon atoms, optionally substituted aryl, optionally substituted arylalkyl, optionally substituted heteroaryl, or optionally substituted heteroarylalkyl.

In certain embodiments at least one of —R¹ or —R² of formula (X) is —CN, —SOR³ or —SO₂R³. In certain embodiments at least one of —R¹ and —R² of formula (X) is —CN or —SO₂R³. In certain embodiments at least one of —R¹ and —R² of formula (X) is —CN or —SO₂R³, wherein —R³ is optionally substituted alkyl, optionally substituted aryl, or —NR⁸². In certain embodiments at least one of —R¹ and —R² of formula (X) is —CN, —SO₂N(CH₃)₂, —SO₂CH₃, phenyl substituted with —SO₂, phenyl substituted with —SO₂ and —Cl, —SO₂N(CH₂CH₂)₂₀, —SO₂CH(CH₃)₂, —SO₂N(CH₃)(CH₂CH₃), or —SO₂N(CH₂CH₂OCH₃)₂.

In certain embodiments each —R⁴ of formula (X) is independently C₁-C₃ alkyl. In certain embodiments both —R⁴ are methyl.

In certain embodiments —Y— of formula (X) is absent. In certain embodiments —Y— of formula (X) is —N(R⁶)CH₂—.

In certain embodiments -L¹- is of formula (X), wherein n is 1, —R¹ is —CN, —R² is —H, and —R⁴ is —CH₃. In certain embodiments -L¹- is of formula (X), wherein n is 1, —R¹ is —SO₂N(CH₃)₂, —R² is —H, and —R⁴ is —CH₃. In certain embodiments -L¹- is of formula (X), wherein n is 1, —R¹ is SO₂CH₃, —R² is —H, and —R⁴ is —CH₃. In certain embodiments -L¹- is of formula (X), wherein n is 1, —R¹ is —SO₂N(CH₂CH₂)₂CHCH₃, —R² is —H, and —R⁴ is —CH₃. In certain embodiments -L¹- is of formula (X), wherein n is 1, —R¹ is phenyl substituted with —SO₂, —R² is —H, and —R⁴ is —CH₃. In certain embodiments -L¹- is of formula (X), wherein n is 1, —R¹ is phenyl substituted with —SO₂ and —Cl, —R² is —H, and —R⁴ is —CH₃. In certain embodiments -L¹- is of formula (X), wherein n is 1, —R¹ is —SO₂N(CH₂CH₂)₂₀, —R² is —H, and —R⁴ is —CH₃. In certain embodiments -L¹- is of formula (X), wherein n is 1, —R¹ is —SO₂CH(CH₃)₂, —R² is —H, and —R⁴ is —CH₃. In certain embodiments -L¹- is of formula (X), wherein n is 1, —R¹ is —SO₂N(CH₃)(CH₂CH₃), —R² is —H, and —R⁴ is —CH₃. In certain embodiments -L¹- is of formula (X), wherein n is 1, —R¹ is —SO₂N(CH₂CH₂OCH₃)₂, —R² is —H, and —R⁴ is —CH₃. In certain embodiments -L¹- is of formula (X), wherein n is 1, —R¹ is phenyl substituted with —SO₂ and —CH₃, —R² is —H, and —R⁴ is —CH₃.

In certain embodiments -L¹- is of formula (X), wherein n is 2, —R¹ is —CN, —R² is —H, and —R⁴ is —CH₃. In certain embodiments -L¹- is of formula (X), wherein n is 2, —R¹ is —SO₂N(CH₃)₂, —R² is —H, and —R⁴ is —CH₃. In certain embodiments -L¹- is of formula (X), wherein n is 2, —R¹ is SO₂CH₃, —R² is —H, and —R⁴ is —CH₃. In certain embodiments -L¹- is of formula (X), wherein n is 2, —R¹ is —SO₂N(CH₂CH₂)₂CHCH₃, —R² is —H, and —R⁴ is —CH₃. In certain embodiments -L¹- is of formula (X), wherein n is 2, —R¹ is phenyl substituted with —SO₂, —R² is —H, and —R⁴ is —CH₃. In certain embodiments -L¹- is of formula (X), wherein n is 2, —R¹ is phenyl substituted with —SO₂ and —Cl, —R² is —H, and —R⁴ is —CH₃. In certain embodiments -L¹- is of formula (X), wherein n is 2, —R¹ is —SO₂N(CH₂CH₂)₂₀, —R² is —H, and —R⁴ is —CH₃. In certain embodiments -L¹- is of formula (X), wherein n is 2, —R¹ is —SO₂CH(CH₃)₂, —R² is —H, and —R⁴ is —CH₃. In certain embodiments -L¹- is of formula (X), wherein n is 2, —R¹ is —SO₂N(CH₃)(CH₂CH₃), —R² is —H, and —R⁴ is —CH₃. In certain embodiments -L¹- is of formula (X), wherein n is 2, —R¹ is —SO₂N(CH₂CH₂OCH₃)₂, —R² is —H, and —R⁴ is —CH₃. In certain embodiments -L¹- is of formula (X), wherein n is 2, —R¹ is phenyl substituted with —SO₂ and —CH₃, —R² is —H, and —R⁴ is —CH₃.

In certain embodiments -L¹- is of formula (X), wherein n is 3, —R¹ is —CN, —R² is —H, and —R⁴ is —CH₃. In certain embodiments -L¹- is of formula (X), wherein n is 3, —R¹ is —SO₂N(CH₃)₂, —R² is —H, and —R⁴ is —CH₃. In certain embodiments -L¹- is of formula (X), wherein n is 3, —R¹ is SO₂CH₃, —R² is —H, and —R⁴ is —CH₃. In certain embodiments -L¹- is of formula (X), wherein n is 3, —R¹ is —SO₂N(CH₂CH₂)₂CHCH₃, —R² is —H, and —R⁴ is —CH₃. In certain embodiments -L¹- is of formula (X), wherein n is 3, —R¹ is phenyl substituted with —SO₂, —R² is —H, and —R⁴ is —CH₃. In certain embodiments -L¹- is of formula (X), wherein n is 3, —R¹ is phenyl substituted with —SO₂ and —Cl, —R² is —H, and —R⁴ is —CH₃. In certain embodiments -L¹- is of formula (X), wherein n is 3, —R¹ is —SO₂N(CH₂CH₂)₂₀, —R² is —H, and —R⁴ is —CH₃. In certain embodiments -L¹- is of formula (X), wherein n is 3, —R¹ is —SO₂CH(CH₃)₂, —R² is —H, and —R⁴ is —CH₃. In certain embodiments -L¹- is of formula (X), wherein n is 3, —R¹ is —SO₂N(CH₃)(CH₂CH₃), —R² is —H, and —R⁴ is —CH₃. In certain embodiments -L¹- is of formula (X), wherein n is 3, —R¹ is —SO₂N(CH₂CH₂OCH₃)₂, —R² is —H, and —R⁴ is —CH₃. In certain embodiments -L¹- is of formula (X), wherein n is 3, —R¹ is phenyl substituted with —SO₂ and —CH₃, —R² is —H, and —R⁴ is —CH₃.

Only in the context of formula (X) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n-octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

In certain embodiments -L²- is a chemical bond. In certain embodiments -L²- is a spacer moiety, such as a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y1a})—, and —OC(O)N(R^{y3})—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R³, —R^{y3a}, R^{y4}, —R^{y4a}, —R^{y5}, —R^{y5a} and —R^{y5}b is independently selected from the group consisting of —H, and C₁₋₆ alkyl, wherein C₁₋₆ alkyl is optionally substituted with one or more halogen, which are the same or different.

In certain embodiments -L²- is selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR)(R^{y3a})—, —N(R^{y3})C(O)N(R^{y3a}) and —OC(O)N(R^{y3})—:
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{5a}R^{y5}b), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4a}, —R^{y4}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—₁—S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
each —R^{y2} is independently selected from the group consisting of halogen, and C₁₋₆ alkyl; and
each —R^{y3}, —R^{y3a}, —R^{y4}, —R^{y4a}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -L²- has a molecular weight in the range of from 14 g/mol to 750 g/mol.

In certain embodiments -L²- comprises a moiety selected from

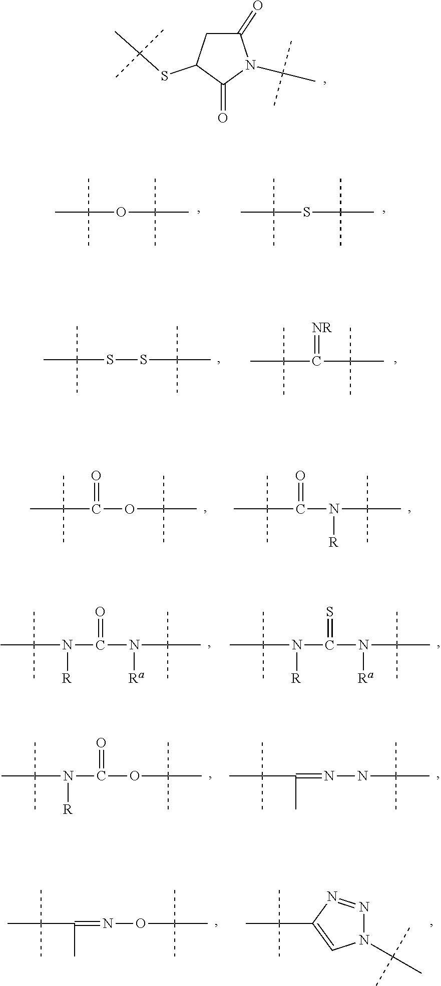

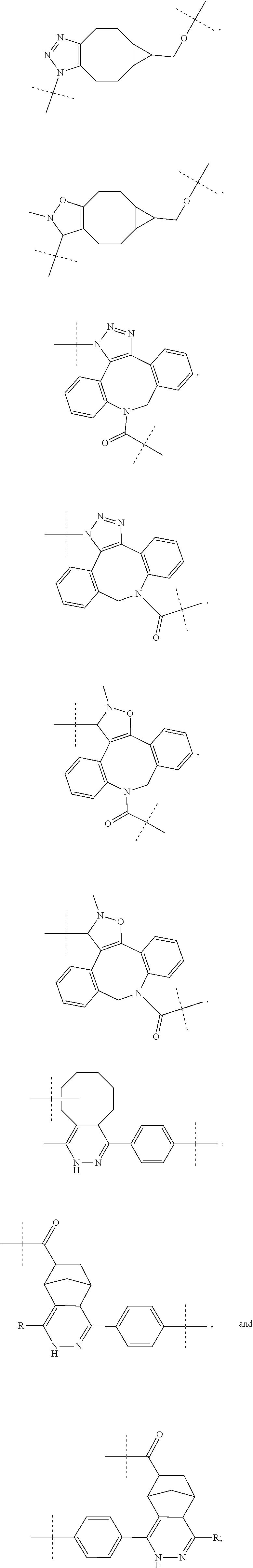

wherein
dashed lines indicate attachment to the remainder of -L²-, -L¹-, —Z and/or Z′, respectively; and
—R and —R^{a} are independently of each other selected from the group consisting of —H, methyl, ethyl, propyl, butyl, pentyl and hexyl.

In certain embodiments -L²- has a chain length of 1 to 20 atoms.

As used herein the term “chain length” with regard to the moiety -L²- refers to the number of atoms of -L²- present in the shortest connection between -L¹- and —Z.

In certain embodiments -L²- is of formula (i)

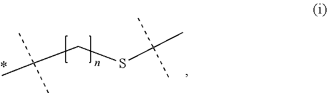

wherein
the dashed line marked with the asterisk indicates attachment to -L¹-;
the unmarked dashed line indicates attachment to —Z or —Z′;
n is selected from the group consisting of 0, 1, 2, 3, 4, 5, 6, 7, 8, 9, 10, 11, 12, 13, 14, 15, 16, 17 and 18; and
wherein the moiety of formula (i) is optionally further substituted.

In certain embodiments n of formula (i) is selected from the group consisting of 3, 4, 5, 6, 7, 8, and 9. In certain embodiments n of formula (i) is 4, 5, 6, or 7. In certain embodiments n of formula (i) is 4. In certain embodiments n of formula (i) is 5. In certain embodiments n of formula (i) is 6.

In certain embodiments the moiety -L¹-L²- is selected from the group consisting of

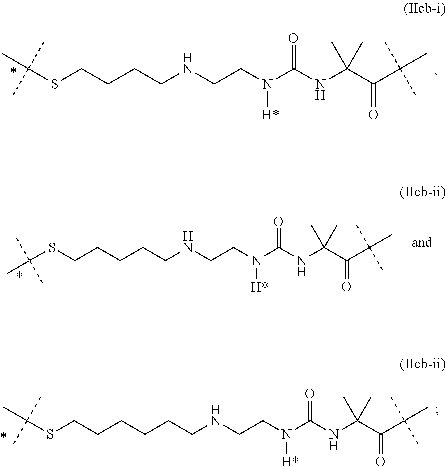

wherein
the unmarked dashed line indicates the attachment to a nitrogen of -D which is a PTH moiety by forming an amide bond; and
the dashed line marked with the asterisk indicates attachment to —Z or Z′.

In certain embodiments the moiety -L¹-L²- is of formula (IIca-ii). In certain embodiments the moiety -L¹-L²- is of formula (IIcb-iii).

In certain embodiments the moiety -L¹-L²- is selected from the group consisting of

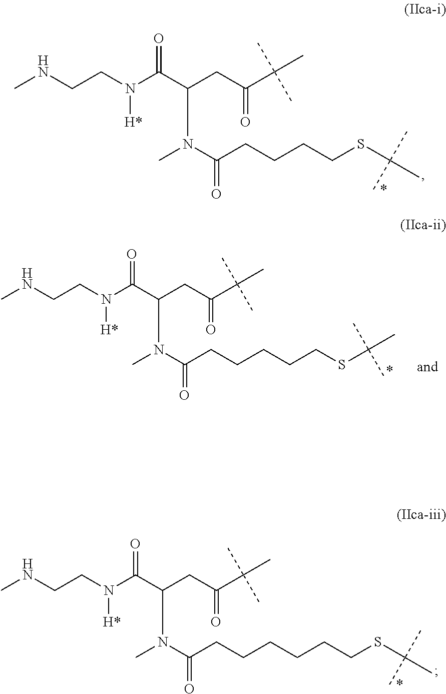

wherein
the unmarked dashed line indicates the attachment to a nitrogen of -D which is a PTH moiety by forming an amide bond; and
the dashed line marked with the asterisk indicates attachment to —Z or Z′.

The carrier —Z comprises a C₈₋₂₄ alkyl or a polymer. In certain embodiments —Z comprises a polymer, such as a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof. In certain embodiments —Z comprises poly(ethylene glycol) (PEG).

In certain embodiments —Z has a molecular weight ranging from 5 to 200 kDa. In certain embodiments —Z has a molecular weight ranging from 8 to 100 kDa, such as ranging from 10 to 80 kDa, from 12 to 60 kDa, or from 15 to 40 kDa. In certain embodiments —Z has a molecular weight of about 20 kDa. In certain embodiments —Z has a molecular weight of about 40 kDa.

In certain embodiments —Z comprises PEG and has a molecular weight ranging from 5 to 200 kDa. In certain embodiments —Z comprises PEG and has a molecular weight ranging from 8 to 100 kDa, such as ranging from 10 to 80 kDa, from 12 to 60 kDa, or from 15 to 40 kDa. In certain embodiments —Z comprises PEG and has a molecular weight of about 20 kDa. In certain embodiments —Z comprises PEG and has a molecular weight of about 40 kDa.

In certain embodiments —Z comprises a protein, such as a protein selected from the group consisting of carboxyl-terminal polypeptide of the chorionic gonadotropin as described in US 2012/0035101 A1 which are herewith incorporated by reference; albumin; XTEN sequences as described in WO 2011123813 A2 which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc fusion proteins. In certain embodiments —Z is a polysarcosine. In certain embodiments —Z comprises a poly(N-methylglycine). In certain embodiments —Z comprises a random coil protein moiety.

In certain embodiments —Z comprises a fatty acid derivate, such as a derivative as disclosed in WO 2005/027978 A2 and WO 2014/060512 A1 which are herewith incorporated by reference.

In certain embodiments —Z is a hyaluronic acid-based polymer.

In certain embodiments —Z is a carrier as disclosed in WO 2012/02047 A1 which is herewith incorporated by reference.

In certain embodiments —Z is a carrier as disclosed in WO 2013/024048 A1 which is herewith incorporated by reference.

In certain embodiments —Z is a PEG-based polymer, such as a linear, branched or multi-arm PEG-based polymer.

In certain embodiments —Z is a linear PEG-based polymer.

In certain embodiments —Z is a multi-arm PEG-based polymer. In certain embodiments —Z is a multi-arm PEG-based polymer having at least 4 PEG-based arms.

In certain embodiments such multi-arm PEG-based polymer —Z is connected to a multitude of moieties -L²-L¹-D, wherein each moiety -L²-L¹-D is in certain embodiments connected to the end of an arm. In certain embodiments such multi-arm PEG-based polymer —Z is connected to 2, 3, 4, 5, 6, 7, 8, 9, 10, 11, 12, 13, 14, 15 or 16 moieties -L²-L¹-D. In certain embodiments such multi-arm PEG-based polymer —Z is connected to 2, 3, 4, 6 or 8 moieties -L²-L¹-D. In certain embodiments such multi-arm PEG-based polymer —Z is connected to 2, 4 or 6 moieties -L²-L¹-D. In certain embodiments such multi-arm PEG-based polymer —Z is connected to 4 or 6 moieties -L²-L¹-D. In certain embodiments such multi-arm PEG-based polymer —Z is connected to 4 moieties -L²-L¹-D.

In certain embodiments —Z is a branched PEG-based polymer. In certain embodiments —Z is a branched PEG-based polymer having one, two, three, four, five or six branching points. In certain embodiments —Z is a branched PEG-based polymer having one, two or three branching points. In certain embodiments —Z is a branched PEG-based polymer having one branching point. In certain embodiments —Z is a branched PEG-based polymer having two branching points. In certain embodiments —Z is a branched PEG-based polymer having three branching points.

In certain embodiments a branching point may be selected from the group consisting of —N<, —CH< and >C<.

In certain embodiments —Z is a branched PEG-based polymer with one branching point and a molecular weight ranging from 5 to 200 kDa. In certain embodiments —Z is a branched PEG-based polymer with one branching point and a molecular weight ranging from 8 to 100 kDa. In certain embodiments —Z is a branched PEG-based polymer with one branching point and a molecular weight ranging from 10 to 80 kDa. In certain embodiments —Z is a branched PEG-based polymer with one branching point and a molecular weight ranging from 12 to 60 kDa. In certain embodiments —Z is a branched PEG-based polymer with one branching point and a molecular weight ranging from 15 to 40 kDa. In certain embodiments —Z is a branched PEG-based polymer with one branching point and a molecular weight of approx. 20 kDa. In certain embodiments —Z is a branched PEG-based polymer with one branching point and a molecular weight of approx. 40 kDa.

In certain embodiments —Z is a branched PEG-based polymer with one branching point, which branching point is —CH<, and a molecular weight of approx. 40 kDa.

In certain embodiments —Z or Z′ comprises a moiety

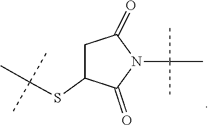

In certain embodiments —Z or Z′ comprises an amide bond.

In certain embodiments-Z comprises a moiety of formula (a)

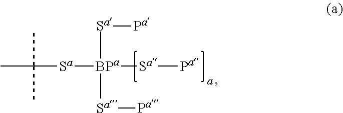

wherein
the dashed line indicates attachment to -L²- or to the remainder of —Z;
BP^{a} is a branching point selected from the group consisting of —N<, —CR< and >C<;
—R is selected from the group consisting of —H and C₁₋₆ alkyl;
a is 0 if BP^{a} is —N< or —CR< and n is 1 if BP^{a} is >C<;
—S^{a}—, —S^{a′}—, —S^{a″}— and —S^{a′″}— are independently of each other a chemical bond or are selected from the group consisting of C₁₋₅₀ alkyl, C₂₋₅₀ alkenyl, and C₂₋₅₀ alkynyl;
wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different; and
P^{a′}, P^{a″} and —P^{a′″} are independently a polymeric moiety.

In certain embodiments BP^{a} of formula (a) is —N<. In certain embodiments BP^{a} of formula (a) is >C<. In certain embodiments BP^{a} of formula (a) is —CR<. In certain embodiments —R is —H.

Accordingly, a of formula (a) is 0.

In certain embodiments —S^{a}— of formula (a) is a chemical bond. In certain embodiments —S^{a}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of -T-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein -T- is a 3- to 10-membered heterocyclyl; and —R⁴ and —R^{4a} are independently selected from the group consisting of —H, methyl, ethyl, propyl and butyl. In certain embodiments —S^{a}— of formula (a) is selected from the group consisting of C₁₋₁₀ alkyl which is interrupted by one or more chemical groups selected from the group consisting of -T-, —C(O)N(R⁴)— and —O—.

In certain embodiments —S^{a′}— of formula (a) is a chemical bond. In certain embodiments —S^{a′}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments —S^{a′}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments —S^{a″}— of formula (a) is a chemical bond. In certain embodiments —S^{a″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments —S^{a″}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments —S^{a′″}— of formula (a) is a chemical bond. In certain embodiments —S^{a′″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments —S^{a′″}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments —P^{a′}, —P^{a″} and —P^{a′″} of formula (a) independently comprise a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments —P^{a′}, —P^{a″} and —P^{a′″} of formula (a) independently comprise a PEG-based moiety. In certain embodiments —P^{a′}, —P^{a″} and —P^{a′″} of formula (a) independently comprise a PEG-based moiety comprising at least 20% PEG, such as at least 30%, such as at least 40% PEG, such as at least 50% PEG, such as at least 60% PEG, such as at least 70% PEG, such as at least 80% PEG or such as at least 90% PEG.

In certain embodiments —P^{a′}, —P^{a″} and —P^{a′″} of formula (a) independently have a molecular weight ranging from and including 5 kDa to 50 kDa, such as from and including 5 kDa to 40 kDa, such as from and including 7.5 kDa to 35 kDa, such as from and 7.5 to 30 kDa or such as from and including 10 to 30 kDa. In certain embodiments —P^{a′}, —P^{a″} and —P^{a′″} of formula (a) have a molecular weight of about 5 kDa. In certain embodiments —P^{a′}, —P^{a″} and —P^{a′″} of formula (a) have a molecular weight of about 7.5 kDa. In certain embodiments —P^{a′}, —P^{a″} and —P^{a′″} of formula (a) have a molecular weight of about 10 kDa. In certain embodiments —P^{a′}, —P^{a″} and —P^{a′″} of formula (a) have a molecular weight of about 12.5 kDa. In certain embodiments —P^{a′}, —P^{a″} and —P^{a′″} of formula (a) have a molecular weight of about 15 kDa. In certain embodiments —P^{a′}, —P^{a″} and —P^{a′″} of formula (a) have a molecular weight of about 20 kDa.

In certain embodiments —Z comprises one moiety of formula (a). In certain embodiments —Z comprises two moieties of formula (a). In certain embodiments —Z comprises three moieties of formula (a). In certain embodiments —Z is a moiety of formula (a).

In certain embodiments —Z comprises a moiety of formula (b)

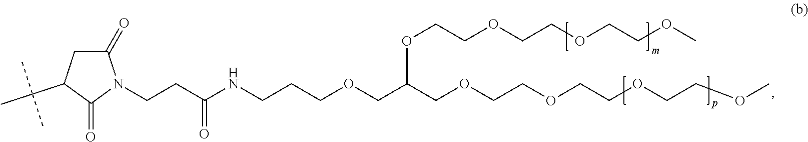

wherein
the dashed line indicates attachment to -L²- or to the remainder of —Z; and
m and p are independently of each other an integer ranging from and including 150 to 1000; such as an integer ranging from and including 150 to 500; such as an integer ranging from and including 200 to 500; or such as an integer ranging from and including 400 to 500.

In certain embodiments m and p of formula (b) are the same integer. In certain embodiments m and p of formula (b) are about 450.

In certain embodiments —Z is a moiety of formula (b).

In certain embodiments the long-acting PTH compound is of formula (IIe-i):

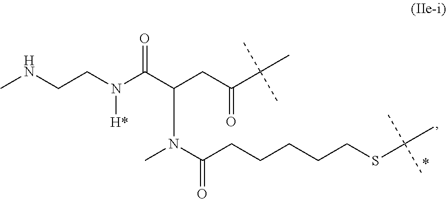

wherein
the unmarked dashed line indicates the attachment to a nitrogen of -D through an amide bond; and
the dashed line marked with the asterisk indicates attachment to a moiety

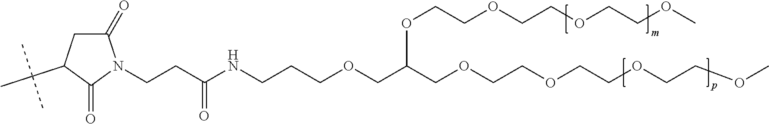

wherein
m and p are independently an integer ranging from and including 400 to 500.

In certain embodiments -D is attached to the compound of formula (IIe-i) through the N-terminal amine functional group of -D.

In certain embodiments the long-acting PTH compound is of formula (IIe-i), wherein the unmarked dashed line indicates attachment to the N-terminal amine of a PTH moiety of SEQ ID NO:51.

In certain embodiments the long-acting PTH compound is of formula (IIe-i), wherein the unmarked dashed line indicates attachment to the N-terminal amine of a PTH moiety of SEQ ID NO:51 and where m and p are approx. 450.

In another preferred embodiment the long-acting PTH compound is a PTH prodrug of formula (IIf-i):

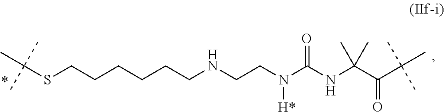

wherein
the unmarked dashed line indicates the attachment to a nitrogen of -D through an amide bond; and
the dashed line marked with the asterisk indicates attachment to a moiety

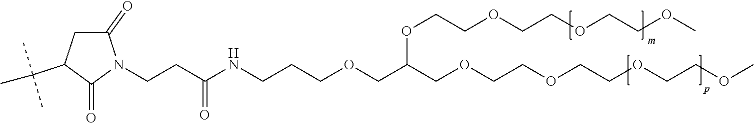

wherein
m and p are independently an integer ranging from and including 400 to 500.

In certain embodiments -D is attached to the compound of formula (IIf-i) through the N-terminal amine functional group of the PTH moiety.

In certain embodiments the long-acting PTH compound is of formula (IIf-i), wherein the unmarked dashed line indicates attachment to the N-terminal amine of a PTH moiety of SEQ ID NO:51.

In certain embodiments the long-acting PTH compound is of formula (IIf-i), wherein the unmarked dashed line indicates attachment to the N-terminal amine of a PTH moiety of SEQ ID NO:51 and wherein both m and p are approx. 450.

In certain embodiments the long-acting PTH compound is of formula (IIf-ii)

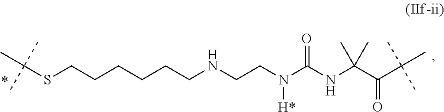

wherein
the unmarked dashed line indicates the attachment to the N-terminal amine of a PTH moiety of SEQ ID NO:51; and
the dashed line marked with the asterisk indicates attachment to a moiety

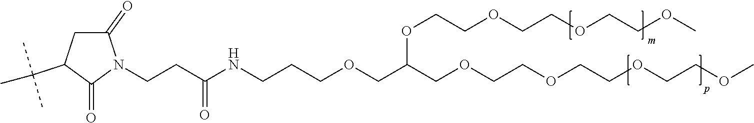

wherein
m and p are independently an integer ranging from and including 400 to 500.

In certain embodiments m and p of formula (IIf-ii) is approx. 450.

In certain embodiments the long-acting PTH compound is of formula (IIf-iii)

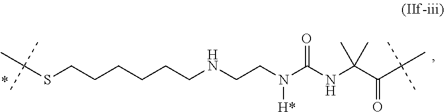

wherein
the unmarked dashed line indicates the attachment to the N-terminal amine of a PTH moiety of SEQ ID NO:51; and
the dashed line marked with the asterisk indicates attachment to a moiety

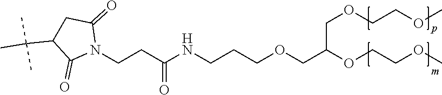

wherein
m and p are independently approx. 450 to 500.

In certain embodiments the long-acting PTH is the polypeptide of SEQ ID NO:122.

In certain embodiments the long-acting PTH compound is k(γE-(miniPEG)₂-γE-COC₁₆H₃₂CO₂H)(N-Me)GSVSEIQLMHNLGKHLNSMERVEWLRKKLQDVHK(γE-(miniPEG)₂-γE-COC₁₆H₃₂CO₂H)—OH, corresponding to SEQ ID NO:87 of WO2021/242756,
wherein
k is d-Lys;
γE is the 1-isomer of gamma, glutamic acid;
miniPEG is COCH₂OCH₂CH₂OCH₂CH₂NH;
COC₁₆H₃₂H is C18 diacid;
(N-Me)G is sarcosine;
SVSEIQLMHNLGKHLNSMERVEWLRKKLQDVHK is PTH (1-32), i.e. SEQ ID NO:53 herein, +K33;
K is 1-Lys; and
OH designates the C-terminal amino acid has a terminal carboxylic acid.

In certain embodiments the long-acting PTH compound is administered to the patient in the form of a pharmaceutical composition comprising one or more long-acting PTH compound as described herein and at least one excipient.

In certain embodiments such pharmaceutical has a pH ranging from and including pH 3 to pH 8. In certain embodiments such pharmaceutical composition has a pH ranging from and including pH 4 to pH 6. In certain embodiments such pharmaceutical composition has a pH ranging from and including pH 4 to pH 5.

In certain embodiments such pharmaceutical composition is a liquid or suspension formulation. It is understood that the pharmaceutical composition is a suspension formulation if the long-acting PTH compound is water-insoluble.

In certain embodiments the pharmaceutical composition is a dry formulation.

Such liquid, suspension or dry pharmaceutical composition comprises at least one excipient.

Excipients used in parenteral formulations may be categorized as, for example, buffering agents, isotonicity modifiers, preservatives, stabilizers, anti-adsorption agents, oxidation protection agents, viscosifiers/viscosity enhancing agents, or other auxiliary agents. However, in some cases, one excipient may have dual or triple functions. In certain embodiments the at least one excipient is selected from the group consisting of
(i) Buffering agents: physiologically tolerated buffers to maintain pH in a desired range, such as sodium phosphate, bicarbonate, succinate, histidine, citrate and acetate, sulphate, nitrate, chloride, pyruvate; antacids such as Mg(OH)₂ or ZnCO₃ may be also used;
(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;
(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride;
(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;
(v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value;
(vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;
(vii) Viscosifiers or viscosity enhancers: in case of a suspension retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);
(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs; and
(ix) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

In certain embodiments 1 ml of the pharmaceutical composition comprises 3456 μg of the long-acting PTH compound of formula (IIf-ii) (corresponding to 300 μg of PTH(1-34)), 1.18 mg succinic acid, 41.7 mg mannitol, 2.5 mg metacresol, 0.13 mg sodium hydroxide and water for injection. In certain embodiments the pharmaceutical composition has a pH of 3.7 to 4.3.

In certain embodiments 1 ml of the pharmaceutical composition comprises 3456 μg of the long-acting PTH compound of formula (IIf-iii) (corresponding to 300 μg of PTH(1-34)), 1.18 mg succinic acid, 41.7 mg mannitol, 2.5 mg metacresol, 0.13 mg sodium hydroxide and water for injection. In certain embodiments the pharmaceutical composition has a pH of 3.7 to 4.3.

In certain embodiments the number of presentations of the pharmaceutical composition ranges from 2 to 8, each presentation comprising a different dose of the long-acting PTH compound. In certain embodiments the number of presentations of the pharmaceutical composition ranges from 2 to 6, each presentation comprising a different dose of the long-acting PTH compound. In certain embodiments the number of presentations of the pharmaceutical composition ranges from 2 to 4, each presentation comprising a different dose of the long-acting PTH compound. In certain embodiments the pharmaceutical composition is provided in 3 presentations, each presentation comprising a different dose of the long-acting PTH compound. In certain embodiments each presentation is a prefilled pen.

In certain embodiments the pharmaceutical composition is provided in 3 presentations, wherein the first presentation is a prefilled pen comprising 168 μg PTH(1-34)/0.56 mL, the second presentation is a prefilled pen comprising 294 μg PTH(1-34)/0.98 mL and the third presentation is a prefilled pen comprising 420 μg PTH(1-34)/1.4 ml. In certain embodiments the first presentation is a prefilled pen comprising 168 μg PTH(1-34)/0.56 mL, the second presentation is a prefilled pen comprising 294 μg PTH(1-34)/0.98 mL and the third presentation is a prefilled pen comprising 420 μg PTH(1-34)/1.4 ml, wherein the PTH(1-34) is provided in the form of the long-acting PTH compound of formula (IIf-ii).

In certain embodiments the pharmaceutical composition is provided in 3 presentations, wherein the first presentation is a prefilled pen comprising 168 μg PTH(1-34)/0.56 mL, the second presentation is a prefilled pen comprising 294 μg PTH(1-34)/0.98 mL and the third presentation is a prefilled pen comprising 420 μg PTH(1-34)/1.4 ml. In certain embodiments the first presentation is a prefilled pen comprising 168 μg PTH(1-34)/0.56 mL, the second presentation is a prefilled pen comprising 294 μg PTH(1-34)/0.98 mL and the third presentation is a prefilled pen comprising 420 μg PTH(1-34)/1.4 ml, wherein the PTH(1-34) is provided in the form of the long-acting PTH compound of formula (IIf-iii).

In certain embodiments the first presentation is used to administer doses of 6, 9 or 12 μg PTH(1-34)/day, the second presentation is used to administer doses of 15, 18 or 21 μg PTH(1-34)/day and the third presentation is used to administer doses of 24, 27 or 30 μg PTH(1-34)/day. Doses higher than 30 and up to and including 60 μg PTH(1-34)/day may be administered as two sequential administrations. For example, a dose of 33 μg PTH(1-34)/day may be administered as a combination of a dose of 15 μg PTH(1-34)/day+a dose of 18 μg PTH(1-34)/day; a dose of 36 μg PTH(1-34)/day may be administered as a combination of a dose of 18 μg PTH(1-34)/day+a dose of 18 μg PTH(1-34)/day; a dose of 39 μg PTH(1-34)/day may be administered as a combination of a dose of 18 μg PTH(1-34)/day+a dose of 21 μg PTH(1-34)/day; a dose of 42 μg PTH(1-34)/day may be administered as a combination of a dose of 21 μg PTH(1-34)/day+a dose of 21 μg PTH(1-34)/day; a dose of 45 μg PTH(1-34)/day may be administered as a combination of a dose of 21 μg PTH(1-34)/day+a dose of 24 μg PTH(1-34)/day; a dose of 48 μg PTH(1-34)/day may be administered as a combination of a dose of 24 μg PTH(1-34)/day+a dose of 24 μg PTH(1-34)/day; a dose of 51 μg PTH(1-34)/day may be administered as a combination of a dose of 24 μg PTH(1-34)/day+a dose of 27 μg PTH(1-34)/day; a dose of 54 μg PTH(1-34)/day may be administered as a combination of a dose of 27 μg PTH(1-34)/day+a dose of 27 μg PTH(1-34)/day; a dose of 57 μg PTH(1-34)/day may be administered as a combination of a dose of 27 μg PTH(1-34)/day+a dose of 30 μg PTH(1-34)/day; and a dose of 60 μg PTH(1-34)/day may be administered as a combination of a dose of 30 μg PTH(1-34)/day+a dose of 30 μg PTH(1-34)/day.

The pharmaceutical composition may be administered to a patient by various modes, such as via topical, enteral or parenteral administration or by methods of external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion, direct delivery to the brain via implanted device allowing delivery of the invention or the like to brain tissue or brain fluids (e.g., Ommaya Reservoir), direct intracerebroventricular injection or infusion, injection or infusion into brain or brain associated regions, injection into the subchoroidal space, retro-orbital injection and ocular instillation. In certain embodiments the pharmaceutical composition is administered via subcutaneous injection.

Injection, such as subcutaneous injection, is in certain embodiments done with a syringe and needle or with a pen injector. In certain embodiments injection, such as subcutaneous injection, is done with a pen injector.

In certain embodiments the long-acting PTH compound, its pharmaceutically acceptable salt or the pharmaceutical composition is administered to the patient once daily. In certain embodiments the long-acting PTH compound, its pharmaceutically acceptable salt or the pharmaceutical composition is administered to the patient every two days. In certain embodiments the long-acting PTH compound, its pharmaceutically acceptable salt or the pharmaceutical composition is administered to the patient every three days. In certain embodiments the long-acting PTH compound, its pharmaceutically acceptable salt or the pharmaceutical composition is administered to the patient every four days. In certain embodiments the long-acting PTH compound, its pharmaceutically acceptable salt or the pharmaceutical composition is administered to the patient every five days. In certain embodiments the long-acting PTH compound, its pharmaceutically acceptable salt or the pharmaceutical composition is administered to the patient every six days. In certain embodiments the long-acting PTH compound, its pharmaceutically acceptable salt or the pharmaceutical composition is administered to the patient once a week.

In a seventh aspect the present invention relates to a pharmaceutical composition comprising the compound of formula (IIf-ii), wherein 1 ml of the pharmaceutical composition comprises 3456 μg of the long-acting PTH compound of formula (IIf-ii) (corresponding to 300 μg of PTH(1-34)), 1.18 mg succinic acid, 41.7 mg mannitol, 2.5 mg metacresol, 0.13 mg sodium hydroxide and water for injection. In certain embodiments the pharmaceutical composition has a pH of 3.7 to 4.3.

In certain embodiments the pharmaceutical composition of the seventh aspect is for use in the treatment of hypoparathyroidism. In certain embodiments the pharmaceutical composition of the seventh aspect is administered via subcutaneous injection. In certain embodiments the pharmaceutical composition of the seventh aspect is administered daily via subcutaneous injection.

In certain embodiments the pharmaceutical composition of the seventh aspect is provided in three presentations, each of which may for example be a prefilled pen. In certain embodiments the first of the three presentation is a prefilled pen comprising 168 μg PTH(1-34)/0.56 mL, the second presentation is a prefilled pen comprising 294 μg PTH(1-34)/0.98 mL and the third presentation is a prefilled pen comprising 420 μg PTH(1-34)/1.4 ml, wherein the PTH(1-34) is provided in the form of the long-acting PTH compound of formula (IIf-ii). In certain embodiments the first presentation is used to administer doses of 6, 9 or 12 μg PTH(1-34)/day, the second presentation is used to administer doses of 15, 18 or 21 μg PTH(1-34)/day and the third presentation is used to administer doses of 24, 27 or 30 μg PTH(1-34)/day. Doses higher than 30 and up to and including 60 μg PTH(1-34)/day may be administered as two sequential administrations. For example, a dose of 33 μg PTH(1-34)/day may be administered as a combination of a dose of 15 μg PTH(1-34)/day+a dose of 18 μg PTH(1-34)/day; a dose of 36 μg PTH(1-34)/day may be administered as a combination of a dose of 18 μg PTH(1-34)/day+a dose of 18 μg PTH(1-34)/day; a dose of 39 μg PTH(1-34)/day may be administered as a combination of a dose of 18 μg PTH(1-34)/day+a dose of 21 μg PTH(1-34)/day; a dose of 42 μg PTH(1-34)/day may be administered as a combination of a dose of 21 μg PTH(1-34)/day+a dose of 21 μg PTH(1-34)/day; a dose of 45 μg PTH(1-34)/day may be administered as a combination of a dose of 21 μg PTH(1-34)/day+a dose of 24 μg PTH(1-34)/day; a dose of 48 μg PTH(1-34)/day may be administered as a combination of a dose of 24 μg PTH(1-34)/day+a dose of 24 μg PTH(1-34)/day; a dose of 51 μg PTH(1-34)/day may be administered as a combination of a dose of 24 μg PTH(1-34)/day+a dose of 27 μg PTH(1-34)/day; a dose of 54 μg PTH(1-34)/day may be administered as a combination of a dose of 27 μg PTH(1-34)/day+a dose of 27 μg PTH(1-34)/day; a dose of 57 μg PTH(1-34)/day may be administered as a combination of a dose of 27 μg PTH(1-34)/day+a dose of 30 μg PTH(1-34)/day; and a dose of 60 μg PTH(1-34)/day may be administered as a combination of a dose of 30 μg PTH(1-34)/day+a dose of 30 μg PTH(1-34)/day.

In an eighth aspect the present invention relates to a pharmaceutical composition comprising the compound of formula (IIf-iii), wherein 1 ml of the pharmaceutical composition comprises 3456 μg of the long-acting PTH compound of formula (IIf-iii) (corresponding to 300 μg of PTH(1-34)), 1.18 mg succinic acid, 41.7 mg mannitol, 2.5 mg metacresol, 0.13 mg sodium hydroxide and water for injection. In certain embodiments the pharmaceutical composition has a pH of 3.7 to 4.3.

In certain embodiments the pharmaceutical composition of the seventh aspect is for use in the treatment of hypoparathyroidism. In certain embodiments the pharmaceutical composition of the seventh aspect is administered via subcutaneous injection. In certain embodiments the pharmaceutical composition of the seventh aspect is administered daily via subcutaneous injection.

In certain embodiments the pharmaceutical composition of the seventh aspect is provided in three presentations, each of which may for example be a prefilled pen. In certain embodiments the first of the three presentation is a prefilled pen comprising 168 μg PTH(1-34)/0.56 mL, the second presentation is a prefilled pen comprising 294 μg PTH(1-34)/0.98 mL and the third presentation is a prefilled pen comprising 420 μg PTH(1-34)/1.4 ml, wherein the PTH(1-34) is provided in the form of the long-acting PTH compound of formula (IIf-iii). In certain embodiments the first presentation is used to administer doses of 6, 9 or 12 μg PTH(1-34)/day, the second presentation is used to administer doses of 15, 18 or 21 μg PTH(1-34)/day and the third presentation is used to administer doses of 24, 27 or 30 μg PTH(1-34)/day. Doses higher than 30 and up to and including 60 μg PTH(1-34)/day may be administered as two sequential administrations. For example, a dose of 33 μg PTH(1-34)/day may be administered as a combination of a dose of 15 μg PTH(1-34)/day+a dose of 18 μg PTH(1-34)/day; a dose of 36 μg PTH(1-34)/day may be administered as a combination of a dose of 18 μg PTH(1-34)/day+a dose of 18 μg PTH(1-34)/day; a dose of 39 μg PTH(1-34)/day may be administered as a combination of a dose of 18 μg PTH(1-34)/day+a dose of 21 μg PTH(1-34)/day; a dose of 42 μg PTH(1-34)/day may be administered as a combination of a dose of 21 μg PTH(1-34)/day+a dose of 21 μg PTH(1-34)/day; a dose of 45 μg PTH(1-34)/day may be administered as a combination of a dose of 21 μg PTH(1-34)/day+a dose of 24 jig PTH(1-34)/day; a dose of 48 μg PTH(1-34)/day may be administered as a combination of a dose of 24 μg PTH(1-34)/day+a dose of 24 μg PTH(1-34)/day; a dose of 51 μg PTH(1-34)/day may be administered as a combination of a dose of 24 μg PTH(1-34)/day+a dose of 27 jig PTH(1-34)/day; a dose of 54 μg PTH(1-34)/day may be administered as a combination of a dose of 27 μg PTH(1-34)/day+a dose of 27 μg PTH(1-34)/day; a dose of 57 μg PTH(1-34)/day may be administered as a combination of a dose of 27 μg PTH(1-34)/day+a dose of 30 μg PTH(1-34)/day; and a dose of 60 μg PTH(1-34)/day may be administered as a combination of a dose of 30 μg PTH(1-34)/day+a dose of 30 μg PTH(1-34)/day.

In a ninth aspect the present invention relates to the compound of formula (IIf-ii) or the pharmaceutical composition of the seventh aspect for use in the treatment of hypoparathyroidism in a patient, wherein the treatment is initiated by
a) confirming that the serum 25(OH) vitamin D of the patient is within the normal range within two weeks before the first dose of the compound of formula (IIf-ii) is administered to the patient and serum calcium is ≥7.8 mg/dL at the initiation of treatment;
b) if the patient is taking active vitamin D at the time treatment with the compound of formula (IIf-ii) is initiated:
   b-i) maintaining the same dose of calcium supplements and discontinue active vitamin D on the same day as the first dose of the compound of formula (IIf-ii) is administered, if serum calcium is ≥8.3 mg/dL; or
   b-ii) reducing the dose of active vitamin D by ≥50% on the same day as the first dose of the compound of formula (IIf-ii) is administered and maintaining the same dose of calcium supplements, if serum calcium is <8.3 mg/dL; or
if the patient is not taking active vitamin D at the time treatment with the compound of formula (IIf-ii) is initiated:
   b-iii) decreasing calcium supplements by at least 1500 mg on the same day as the first dose of the compound of formula (IIf-ii) is administered; and
c) optionally continuing dietary calcium supplements at doses of ≤600 mg/day, if calcium supplements are indicated to meet dietary requirements.

In certain embodiments serum 25(OH) vitamin D is within the normal range if its concentration is between 20 and 80 ng/ml.

If the patient is not taking active vitamin D and if calcium supplement doses of ≤1500 mg calcium/day are taken, calcium supplement doses are discontinued entirely in step b-iii).

In certain embodiments the treatment of the eighth aspect comprises in addition to the initiation of the treatment as described above the steps of
d) administering a starting dose of 18 μg PTH(1-34)/day in the form of the compound of formula (IIf-ii), followed by daily administrations of the same dose;
e) measuring serum calcium within 7 to 14 days of the first administration of the compound of formula (IIf-ii); and
f) adjusting the dose of the compound of formula (IIf-ii), active vitamin D and/or calcium supplement.

In certain embodiments the compound of formula (IIf-ii) is administered in step d) as a pharmaceutical composition, of which each ml comprises 3456 μg of the compound of formula (IIf-ii), which corresponds to 300 μg of PTH(1-34), 1.18 mg succinic acid, 41.7 mg mannitol, 2.5 mg metacresol, 0.13 mg sodium hydroxide, and water for injection. In certain embodiments the pharmaceutical composition has a pH of 3.7 to 4.3.

Adjusting the dose of the compound of formula (IIf-ii), active vitamin D and/or calcium supplement in step f) is performed based on the serum calcium levels from step e).

In certain embodiments adjustments in the dose of the compound of formula (IIf-ii), active vitamin D and/or calcium supplement of step f) are performed as follows:
f-i) if serum calcium is <8.3 mg/dL:
   if ≥7 days have passed since treatment with the compound of formula (IIf-ii) was started or the dose of the compound of formula (IIf-ii) was changed, the same calcium supplement and active vitamin D doses are continued, and the dose of the compound of formula (IIf-ii) is increased by 3 μg; or
   if fewer than 7 days have passed since treatment with the compound of formula (IIf-ii) has started or the dose of the compound of formula (IIf-ii) was changed, calcium supplements and/or active vitamin D are increased toward prior doses based on physician's clinical judgement and the same dose of the compound of formula (IIf-ii) is continued;
f-ii) if serum calcium ranges from 8.3 to 10.6 mg/dL:
   if ≥7 days have passed since treatment with the compound of formula (IIf-ii) was started or since the dose of the compound of formula (IIf-ii) was changed and the patient is still taking active vitamin D, active vitamin D is discontinued, and the dose of the compound of formula (IIf-ii) is increased by 3 μg;
   if ≥7 days have passed since treatment with the compound of formula (IIf-ii) was started or since the dose of the compound of formula (IIf-ii) was changed and the patient is no longer taking active vitamin D, but is taking calcium supplements, wherein the calcium supplement is ≥1500 mg/day, the calcium supplement is decreased by ≥1500 mg and the dose of the compound of formula (IIf-ii) is increased by 3 μg;
   if ≥7 days have passed since treatment with the compound of formula (IIf-ii) was started or since the dose of the compound of formula (IIf-ii) was changed and the patient is no longer taking active vitamin D, but is taking calcium supplements, wherein the calcium supplement is less than 1500 mg/day, calcium supplements are discontinued, and the dose of the compound of formula (IIf-ii) is increased by 3 μg;
   if ≥7 days have passed since treatment with the compound of formula (IIf-ii) was started or since the dose of the compound of formula (IIf-ii) was changed and the patient is no longer taking active vitamin D and is no longer taking calcium supplements, the same dose of the compound of formula (IIf-ii) is continued; or
   if fewer than 7 days have passed since treatment with the compound of formula (IIf-ii) was started or since the dose of the compound of formula (IIf-ii) was changed, the same dose of the compound of formula (IIf-ii), of the calcium supplement and active vitamin D is continued;
f-iii) if serum calcium ranges from 10.7 to 11.9 mg/dL:
   if the patient is still taking active vitamin D, active vitamin D is discontinued and the same doses of the compound of formula (IIf-ii) and calcium supplement are continued;
   if the patient is not taking active vitamin D but takes calcium supplements at a dose of ≥1500 mg/day, calcium supplements are decreased by ≥1500 mg and the same dose of the compound of formula (IIf-ii) is continued;
   if the patient is not taking active vitamin D but takes calcium supplements, which calcium supplements are less than 1500 mg per day, calcium supplements are discontinued, and the same dose of the compound of formula (IIf-ii) is continued; or
   if the patient is not taking active vitamin D and does not take calcium supplements, the dose of the compound of formula (If-ii) is decreased by 3 μg; or
f-iv) if serum calcium is >12 mg/dL:
   withhold the compound of formula (IIf-ii) for 2 to 3 days, recheck serum calcium and
      if subsequent serum calcium is <12 mg/dL, resume titration of the compound of formula (IIf-ii), active vitamin D and calcium supplements as described in steps f-i) to f-iii) using the most recent serum calcium value obtained; and
      if serum calcium remains ≥12 mg/dL, withhold the compound of formula (IIf-ii) for an additional 2 to 3 days, recheck serum calcium and proceed as described in the previous step.

In certain embodiments the dosage range of the compound of formula (IIf-ii) administered to the patient ranges from and includes 6 to 60 μg PTH(1-34)/day, which doses may be provided in prefilled pens of 168 μg PTH(1-34)/0.56 mL (delivering doses of 6, 9 or 12 μg PTH(1-34)); 294 μg PTH(1-34)/0.98 mL (delivering doses of 15, 18, or 21 μg PTH(1-34)); and 420 μg PTH(1-34)/1.4 ml (delivering doses of 24, 27, or 30 μg PTH(1-34)).

In certain embodiments the compound of formula (IIf-ii) or the pharmaceutical composition comprising the compound of formula (IIf-ii) is inspected visually for particulate matter and discoloration prior to administration.

In certain embodiments the compound of formula (IIf-ii) or the pharmaceutical composition comprising the compound of formula (IIf-ii) is administered subcutaneously daily to the abdomen or front of the thigh and the injection site is rotated daily. For doses of >30 μg PTH(1-34)/day two sequential injections are required, using different sites for each injection. For example, a dose of 33 μg PTH(1-34)/day may be administered as a combination of a dose of 15 μg PTH(1-34)/day+a dose of 18 μg PTH(1-34)/day; a dose of 36 μg PTH(1-34)/day may be administered as a combination of a dose of 18 μg PTH(1-34)/day+a dose of 18 μg PTH(1-34)/day; a dose of 39 μg PTH(1-34)/day may be administered as a combination of a dose of 18 μg PTH(1-34)/day+a dose of 21 μg PTH(1-34)/day; a dose of 42 μg PTH(1-34)/day may be administered as a combination of a dose of 21 μg PTH(1-34)/day+a dose of 21 μg PTH(1-34)/day; a dose of 45 μg PTH(1-34)/day may be administered as a combination of a dose of 21 μg PTH(1-34)/day+a dose of 24 μg PTH(1-34)/day; a dose of 48 μg PTH(1-34)/day may be administered as a combination of a dose of 24 μg PTH(1-34)/day+a dose of 24 μg PTH(1-34)/day; a dose of 51 μg PTH(1-34)/day may be administered as a combination of a dose of 24 μg PTH(1-34)/day+a dose of 27 μg PTH(1-34)/day; a dose of 54 μg PTH(1-34)/day may be administered as a combination of a dose of 27 μg PTH(1-34)/day+a dose of 27 μg PTH(1-34)/day; a dose of 57 μg PTH(1-34)/day may be administered as a combination of a dose of 27 μg PTH(1-34)/day+a dose of 30 μg PTH(1-34)/day; and a dose of 60 μg PTH(1-34)/day may be administered as a combination of a dose of 30 μg PTH(1-34)/day+a dose of 30 μg PTH(1-34)/day. It is understood that in these examples the PTH(1-34) is administered in the form of the compound of formula (IIf-ii).

In certain embodiments dose adjustments of the compound of formula (IIf-ii), active vitamin D and calcium supplements are made on the same day. After a dose change in the compound of formula (IIf-ii), active vitamin D or calcium supplements, serum calcium is in certain embodiments measured within 7 to 14 days and the patient may be monitored for clinical symptoms of hypocalcemia or hypercalcemia and doses of the compound of formula (IIf-ii), active vitamin D and/or calcium supplements may be adjusted as described above.

The dose of the compound of formula (IIf-ii) may be increased as described above in increments of 3 μg if at least 7 days have elapsed since a prior dose change of the compound of formula (IIf-ii). In certain embodiments the dose of the compound of formula (IIf-ii) administered to the patient is no more often than every 7 days adjusted. The dose of the compound of formula (IIf-ii) may be reduced no more often than every 3 days in 3 μg increments in response to hypercalcemia as described above.

The maintenance dose should be the dose of the compound of formula (IIf-ii) that achieves serum calcium within the normal range, without the need for active vitamin D or therapeutic doses of calcium. Optionally, calcium supplementation sufficient to meet dietary requirements may be continued. Serum calcium may be measured per standard of care once a maintenance dose is achieved.

Accordingly, the treatment of the eighth aspect comprises in addition to steps a) to f) as described above the steps of
g) administration of a daily maintenance dose; and
h) measuring serum calcium per standard of care.

Optionally, step h) is followed by repeating steps f) to h), if serum calcium levels are not within the normal range, such as within a range from 8.3 to 10.6 mg/dL.

In certain embodiments the daily maintenance dose is administered for at least one week, such as for two weeks, for three weeks, for four weeks, for five weeks, for six weeks, for seven weeks, for eight weeks, for ten weeks or for twelve weeks. It is understood that changes in factors such as for example extend of physical activity or diet may lead to a change in the daily maintenance dose needed.

If a dose is missed by less than 12 hours, it may be taken as soon as possible. If a dose is missed by more than 12 hours, it may be skipped, and the next dose is then taken as scheduled. If administration of 3 or more consecutive doses is missed, it is recommended to monitor for signs and symptoms of hypocalcemia and to consider measuring serum calcium. If indicated, treatment with calcium supplements and active vitamin D may be resumed. In certain embodiments administration of the compound of formula (IIf-ii) is resumed at the prescribed dose as soon as possible after an interruption, which prescribed dose may be the maintenance dose or not. When resuming treatment after an interruption, serum calcium should be measured and doses of the compound of formula (IIf-ii), active vitamin D and calcium supplements may be adjusted as described above.

In a tenth aspect the present invention relates to the compound of formula (IIf-iii) or the pharmaceutical composition of the seventh aspect for use in the treatment of hypoparathyroidism in a patient, wherein the treatment is initiated by
a) confirming that the serum 25(OH) vitamin D of the patient is within the normal range within two weeks before the first dose of the compound of formula (IIf-iii) is administered to the patient and serum calcium is ≥7.8 mg/dL at the initiation of treatment;
b) if the patient is taking active vitamin D at the time treatment with the compound of formula (IIf-iii) is initiated:
   b-i) maintaining the same dose of calcium supplements and discontinue active vitamin D on the same day as the first dose of the compound of formula (IIf-iii) is administered, if serum calcium is ≥8.3 mg/dL; or
   b-ii) reducing the dose of active vitamin D by ≥50% on the same day as the first dose of the compound of formula (IIf-iii) is administered and maintaining the same dose of calcium supplements, if serum calcium is <8.3 mg/dL; or
if the patient is not taking active vitamin D at the time treatment with the compound of formula (IIf-iii) is initiated:
   b-iii) decreasing calcium supplements by at least 1500 mg on the same day as the first dose of the compound of formula (IIf-iii) is administered; and
c) optionally continuing dietary calcium supplements at doses of ≤600 mg/day, if calcium supplements are indicated to meet dietary requirements.

In certain embodiments serum 25(OH) vitamin D is within the normal range if its concentration is between 20 and 80 ng/ml.

If the patient is not taking active vitamin D and if calcium supplement doses of ≤1500 mg calcium/day are taken, calcium supplement doses are discontinued entirely in step b-iii).

In certain embodiments the treatment of the eighth aspect comprises in addition to the initiation of the treatment as described above the steps of
d) administering a starting dose of 18 μg PTH(1-34)/day in the form of the compound of formula (IIf-iii), followed by daily administrations of the same dose;
e) measuring serum calcium within 7 to 14 days of the first administration of the compound of formula (IIf-iii); and
f) adjusting the dose of the compound of formula (IIf-iii), active vitamin D and/or calcium supplement.

In certain embodiments the compound of formula (IIf-iii) is administered in step d) as a pharmaceutical composition, of which each ml comprises 3456 μg of the compound of formula (IIf-iii), which corresponds to 300 μg of PTH(1-34), 1.18 mg succinic acid, 41.7 mg mannitol, 2.5 mg metacresol, 0.13 mg sodium hydroxide, and water for injection. In certain embodiments the pharmaceutical composition has a pH of 3.7 to 4.3.

Adjusting the dose of the compound of formula (IIf-iii), active vitamin D and/or calcium supplement in step f) is performed based on the serum calcium levels from step e).

In certain embodiments adjustments in the dose of the compound of formula (IIf-iii), active vitamin D and/or calcium supplement of step f) are performed as follows:
f-i) if serum calcium is <8.3 mg/dL:
   if ≥7 days have passed since treatment with the compound of formula (IIf-iii) was started or the dose of the compound of formula (IIf-iii) was changed, the same calcium supplement and active vitamin D doses are continued, and the dose of the compound of formula (IIf-iii) is increased by 3 μg; or
   if fewer than 7 days have passed since treatment with the compound of formula (IIf-iii) has started or the dose of the compound of formula (IIf-iii) was changed, calcium supplements and/or active vitamin D are increased toward prior doses based on physician's clinical judgement and the same dose of the compound of formula (IIf-iii) is continued;
f-ii) if serum calcium ranges from 8.3 to 10.6 mg/dL:
   if ≥7 days have passed since treatment with the compound of formula (IIf-iii) was started or since the dose of the compound of formula (IIf-iii) was changed and the patient is still taking active vitamin D, active vitamin D is discontinued, and the dose of the compound of formula (IIf-iii) is increased by 3 μg;
   if ≥7 days have passed since treatment with the compound of formula (IIf-iii) was started or since the dose of the compound of formula (IIf-iii) was changed and the patient is no longer taking active vitamin D, but is taking calcium supplements, wherein the calcium supplement is ≥1500 mg/day, the calcium supplement is decreased by 1500 mg and the dose of the compound of formula (IIf-iii) is increased by 3 μg;
   if ≥7 days have passed since treatment with the compound of formula (IIf-iii) was started or since the dose of the compound of formula (IIf-iii) was changed and the patient is no longer taking active vitamin D, but is taking calcium supplements, wherein the calcium supplement is less than 1500 mg/day, calcium supplements are discontinued, and the dose of the compound of formula (IIf-iii) is increased by 3 μg;
   if ≥7 days have passed since treatment with the compound of formula (IIf-iii) was started or since the dose of the compound of formula (IIf-iii) was changed and the patient is no longer taking active vitamin D and is no longer taking calcium supplements, the same dose of the compound of formula (IIf-iii) is continued; or
   if fewer than 7 days have passed since treatment with the compound of formula (IIf-iii) was started or since the dose of the compound of formula (IIf-iii) was changed, the same dose of the compound of formula (IIf-iii), of the calcium supplement and active vitamin D is continued;
f-iii) if serum calcium ranges from 10.7 to 11.9 mg/dL:
   if the patient is still taking active vitamin D, active vitamin D is discontinued and the same doses of the compound of formula (IIf-iii) and calcium supplement are continued;
   if the patient is not taking active vitamin D but takes calcium supplements at a dose of ≥1500 mg/day, calcium supplements are decreased by ≥1500 mg and the same dose of the compound of formula (IIf-iii) is continued;
   if the patient is not taking active vitamin D but takes calcium supplements, which calcium supplements are less than 1500 mg per day, calcium supplements are discontinued, and the same dose of the compound of formula (IIf-iii) is continued; or
   if the patient is not taking active vitamin D and does not take calcium supplements, the dose of the compound of formula (IIf-iii) is decreased by 3 μg; or
f-iv) if serum calcium is ≥12 mg/dL:
   withhold the compound of formula (IIf-iii) for 2 to 3 days, recheck serum calcium and
      if subsequent serum calcium is <12 mg/dL, resume titration of the compound of formula (IIf-iii), active vitamin D and calcium supplements as described in steps f-i) to f-iii) using the most recent serum calcium value obtained; and
      if serum calcium remains ≥12 mg/dL, withhold the compound of formula (IIf-iii) for an additional 2 to 3 days, recheck serum calcium and proceed as described in the previous step.

In certain embodiments the dosage range of the compound of formula (IIf-iii) administered to the patient ranges from and includes 6 to 60 μg PTH(1-34)/day, which doses may be provided in prefilled pens of 168 μg PTH(1-34)/0.56 mL (delivering doses of 6, 9 or 12 μg PTH(1-34)); 294 μg PTH(1-34)/0.98 mL (delivering doses of 15, 18, or 21 μg PTH(1-34)); and 420 μg PTH(1-34)/1.4 ml (delivering doses of 24, 27, or 30 μg PTH(1-34)).

In certain embodiments the compound of formula (IIf-iii) or the pharmaceutical composition comprising the compound of formula (IIf-iii) is inspected visually for particulate matter and discoloration prior to administration.

In certain embodiments the compound of formula (IIf-iii) or the pharmaceutical composition comprising the compound of formula (IIf-iii) is administered subcutaneously daily to the abdomen or front of the thigh and the injection site is rotated daily. For doses of >30 μg PTH(1-34)/day two sequential injections are required, using different sites for each injection. For example, a dose of 33 μg PTH(1-34)/day may be administered as a combination of a dose of 15 μg PTH(1-34)/day+a dose of 18 μg PTH(1-34)/day; a dose of 36 μg PTH(1-34)/day may be administered as a combination of a dose of 18 μg PTH(1-34)/day+a dose of 18 μg PTH(1-34)/day; a dose of 39 μg PTH(1-34)/day may be administered as a combination of a dose of 18 μg PTH(1-34)/day+a dose of 21 μg PTH(1-34)/day; a dose of 42 μg PTH(1-34)/day may be administered as a combination of a dose of 21 μg PTH(1-34)/day+a dose of 21 μg PTH(1-34)/day; a dose of 45 μg PTH(1-34)/day may be administered as a combination of a dose of 21 μg PTH(1-34)/day+a dose of 24 μg PTH(1-34)/day; a dose of 48 μg PTH(1-34)/day may be administered as a combination of a dose of 24 μg PTH(1-34)/day+a dose of 24 μg PTH(1-34)/day; a dose of 51 μg PTH(1-34)/day may be administered as a combination of a dose of 24 μg PTH(1-34)/day+a dose of 27 μg PTH(1-34)/day; a dose of 54 μg PTH(1-34)/day may be administered as a combination of a dose of 27 μg PTH(1-34)/day+a dose of 27 μg PTH(1-34)/day; a dose of 57 μg PTH(1-34)/day may be administered as a combination of a dose of 27 μg PTH(1-34)/day+a dose of 30 μg PTH(1-34)/day; and a dose of 60 μg PTH(1-34)/day may be administered as a combination of a dose of 30 μg PTH(1-34)/day+a dose of 30 μg PTH(1-34)/day. It is understood that in these examples the PTH(1-34) is administered in the form of the compound of formula (IIf-iii).

In certain embodiments dose adjustments of the compound of formula (IIf-iii), active vitamin D and calcium supplements are made on the same day. After a dose change in the compound of formula (IIf-iii), active vitamin D or calcium supplements, serum calcium is in certain embodiments measured within 7 to 14 days and the patient may be monitored for clinical symptoms of hypocalcemia or hypercalcemia and doses of the compound of formula (IIf-iii), active vitamin D and/or calcium supplements may be adjusted as described above.

The dose of the compound of formula (IIf-iii) may be increased as described above in increments of 3 μg if at least 7 days have elapsed since a prior dose change of the compound of formula (IIf-iii). In certain embodiments the dose of the compound of formula (IIf-iii) administered to the patient is no more often than every 7 days adjusted. The dose of the compound of formula (IIf-iii) may be reduced no more often than every 3 days in 3 μg increments in response to hypercalcemia as described above.

The maintenance dose should be the dose of the compound of formula (IIf-iii) that achieves serum calcium within the normal range, without the need for active vitamin D or therapeutic doses of calcium. Optionally, calcium supplementation sufficient to meet dietary requirements may be continued. Serum calcium may be measured per standard of care once a maintenance dose is achieved.

Accordingly, the treatment of the eighth aspect comprises in addition to steps a) to f) as described above the steps of
g) administration of a daily maintenance dose; and
h) measuring serum calcium per standard of care.

Optionally, step h) is followed by repeating steps f) to h), if serum calcium levels are not within the normal range, such as within a range from 8.3 to 10.6 mg/dL.

In certain embodiments the daily maintenance dose is administered for at least one week, such as for two weeks, for three weeks, for four weeks, for five weeks, for six weeks, for seven weeks, for eight weeks, for ten weeks or for twelve weeks. It is understood that changes in factors such as for example extend of physical activity or diet may lead to a change in the daily maintenance dose needed.

If a dose is missed by less than 12 hours, it may be taken as soon as possible. If a dose is missed by more than 12 hours, it may be skipped, and the next dose is then taken as scheduled. If administration of 3 or more consecutive doses is missed, it is recommended to monitor for signs and symptoms of hypocalcemia and to consider measuring serum calcium. If indicated, treatment with calcium supplements and active vitamin D may be resumed. In certain embodiments administration of the compound of formula (IIf-iii) is resumed at the prescribed dose as soon as possible after an interruption, which prescribed dose may be the maintenance dose or not. When resuming treatment after an interruption, serum calcium should be measured and doses of the compound of formula (IIf-iii), active vitamin D and calcium supplements may be adjusted as described above.

In certain embodiments the patient of the ninth and tenth aspect is a mammal. In certain embodiments the patient of the ninth and tenth aspect is a non-human primate. In certain embodiments the patient of the ninth and tenth aspect is a human patient. In certain embodiments the patient of the ninth and tenth aspect is an adult human patient. In certain embodiments the patient of the ninth and tenth aspect is a pediatric human patient.

### Material

Compound 1 has the following structure:

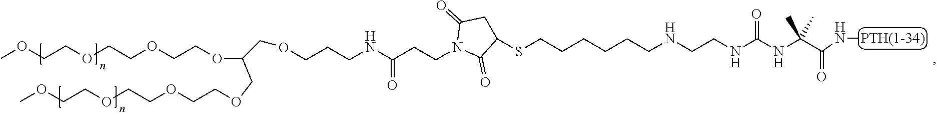

wherein the PTH(1-34) moiety has the sequence of SEQ ID NO:51 and is attached to the remainder of the PTH compound via the nitrogen of the N-terminal amine by forming an amide bond and wherein each n is approx. 450. It is understood that the nitrogen immediately left of “PTH(1-34)” corresponds to the nitrogen of the N-terminal amine.

Compound 1 is obtainable from the method described in WO 2018/060312 A1 for compound 18. Compound 1 is also known as “TransCon PTH”.

### EXAMPLE 1

### Trial Design

Compound 1 was studied in a phase 2, multicenter, randomized, double-blind, placebo-controlled, parallel-group, 4-week clinical trial with an open-label extension period (OLE). The protocol was reviewed by the appropriate institutional review boards and independent ethics committees, with participants providing signed informed consent prior to initiation (ClinicalTrials.gov identifier: NCT04009291; EudraCT No.: 2018-004815-33).

### Population

Men and nonpregnant female adults aged 18 years and older with a body mass index of 17 to 40 and postsurgical, autoimmune, genetic, or idiopathic hypoparathyroidism were enrolled. Hypoparathyroidism diagnosis was based on hypocalcemia in the setting of inappropriately low serum PTH levels of at least 26 weeks' duration. Participants were on stable doses of active vitamin D (defined as calcitriol ≥0.5 μg/day, alfacalcidol ≥1.0 μg/day) and calcium (defined as elemental calcium ≥800 mg/day) for at least 12 weeks before screening and were required to have an estimated glomerular filtrate rate of at least 30 mL/min/1.73 m². Individuals with mutations in the calcium-sensing receptor gene were excluded, as were people with pseudohypoparathyroidism, diseases other than hypoparathyroidism affecting calcium or PTH homeostasis (including hyperparathyroidism, Paget disease, hypomagnesemia, type 1 or poorly controlled type 2 diabetes mellitus, Cushing syndrome, or multiple endocrine neoplasia), or other significant comorbidities. Individuals taking certain medications (loop or thiazide diuretics, phosphate binders, bisphosphonates, PTH-like or other drugs known to influence calcium and bone metabolism except for calcium supplements and active vitamin D analogs) were also excluded. Table 1 shows baseline demographics of the study.

TABLE 1
Baseline demographics

| | All Subjects | |
|---|---|---|
| | (N = 59) | |
| Age (years), mean (SD) | 50 | (12) |
| Age group (years), % | | |
| <30 | 5 | |
| ≥30-<65 | 86 | |
| ≥65 | 9 | |
| Sex, % | | |
| Female | 81 | |
| Race, % | | |
| White | 92 | |
| Geographical Region, % | | |
| North America | 64 | |
| Europe | 36 | |
| Cause of HP, % | | |
| Acquired from neck surgery | 80 | |
| Autoimmune disease | 2 | |
| Idiopathic disease | 19 | |
| Duration of HP (years), mean (range) | 12 | (1-39) |

| | |
|---|---|
| Calcium | |
| Mean TDD (mg) | 2079 |
| Calcium ≤1000 mg TDD, % | 19 |
| Calcium ≤2000 mg TDD, % | 63 |
| Calcitriol (Active Vitamin D), % | 76 |
| Mean TDD (μg) | 0.79 |
| Alfacalcidol (Active Vitamin D), % | 22 |
| Mean TDD (μg) | 2.38 |

### Trial Protocol

During the screening period and before randomization, active vitamin D and calcium supplementation was optimized to achieve a 25-hyroxyvitamin D level between 30 and 70 ng/mL, normal serum magnesium, and an albumin-adjusted (or ionized) serum calcium (sCa) level in the lower half of normal. For the purpose of this trial, the normal range for albumin-adjusted sCa was 8.3 to 10.6 mg/dL (2.07-2.64 mmol/L) and the normal range for ionized sCa was 1.16-1.32 mmol/L. Participants were then randomly assigned to receive daily Compound 1 at 15, 18 or 21 μg PTH(1-34)/day, or placebo (sub-randomized into 3 groups of 1:1:1 to mimic the 3 Compound 1 cohorts). The drug was administered by subcutaneous (SC) injection via prefilled pens with a 31-gauge needle. Participants were maintained at the same dose throughout the 4-week blinded period. When sCa was within the normal range at predefined visits, active vitamin D was decreased by increments of 33% to 50% (ie, by skipping the second of 2 daily doses or skipping the third of 3 daily doses) until discontinued. Calcium was subsequently decreased and ultimately discontinued according to a predefined protocol. In the event of laboratory results or clinical symptoms indicating hypocalcemia, rescue doses of active vitamin D and/or calcium were permitted. For the open-label extension period, participants were assigned to treatment groups based on the continued need for active vitamin D. Individuals no longer requiring active vitamin D were continued on the same dose of Compound 1 as previously. Those still receiving active vitamin D were started on a Compound 1 dose of 15 μg/day with the titration of active vitamin D and calcium as per the initial 4-week period protocol. At follow-up visits, Compound 1 was either increased by 3 μg/day (if persistently hypocalcemic or if sCa was below the lower limit of normal), maintained at the same dose, or decreased by 3 μg/day (if persistently hypercalcemic and no longer taking active vitamin D or calcium). Doses for the extension period for Compound 1 ranged from 6 to 60 μg/day, with rescue doses of active vitamin D and/or calcium allowed throughout.

### Bone Markers

Bone markers P1NP and CTx were measured as described in J Bone Miner Res. 2019 August; 34(8): 1436-1445. doi:10.1002/jbmr.3715.

Changes in bone turnover markers are shown in Table 2. At week 26 mean values for both the anabolic marker P1NP and resorptive marker CTx increased within the normal range or to just above the normal range, respectively, reflecting exposure to the physiologic bone-remodeling effects of PTH. At week 58, the levels of anabolic and catabolic bone markers were trending to mid-normal levels, a trend that continued through week 110.

TABLE 2
Changes in bone turnover markers. “All Compound 1” indicates data
from open-label extension study, in which all participants obtained Compound 1.

| | P1NP | | | CTX | | |
|---|---|---|---|---|---|---|
| | Compound 1 | | | Compound 1 | | |
| Time | ng/mL | | All | ng/L | | All |
| (weeks) | (+/−SD) | Placebo | Compound 1 | (+/−SD) | Placebo | Compound 1 |
| 0 | 33.75 | 40.29 | | 195.35 | 224.00 | |
| | (2.27) | (8.31) | | (17.57) | (27.36) | |
| 2 | 24.89 | 39.92 | | 425.12 | 244.67 | |
| | (1.71) | (7.00) | | (47.60) | (29.66) | |
| 4 | 34.96 | 40.69 | 36.42 | 527.05 | 224.67 | 450.17 |
| | (2.72) | (6.74) | (2.65) | (46.37) | (29.66) | (39.27) |
| 8 | | | 60.39 | | | 774.73 |
| | | | (5.04) | | | (75.07) |
| 12 | | | 76.76 | | | 804.51 |
| | | | (6.15) | | | (74.62) |
| 26 | | | 88.35 | | | 762.98 |
| | | | (5.82) | | | (55.59) |
| 42 | | | 80.73 | | | 610.00 |
| | | | (10.45) | | | (104.07) |
| 58 | | | 70.64 | | | 524.91 |
| | | | (3.80) | | | (42.55) |
| 110 | | | 58.33 | | | 449.46 |
| | | | (3.78) | | | (33.44) |

### BMD Measurements Via DXA

To evaluate bone density and quality, a DXA scan of the spine, hip, and forearm, as well as trabecular bone score (TBS) scoring was performed. TBS scoring was not be performed for subjects 18 to 20 years of age. The D×A images was read centrally by an external vendor. Whenever possible, the same DXA machine was used throughout the trial. Results are shown in Table 3.

TABLE 3
DXA measurements

| | Group A*- Week 26 | | | Group B* - Week 58 | | |
|---|---|---|---|---|---|---|
| | | | Week 26 | | | Week 58 |
| Anatomic | | | change from | | | change from |
| region | Baseline | Week 26 | baseline | Baseline | Week 58 | baseline |
| Lumbar spine | n = 42 | | | n = 42 | | |
| L1-L4 | | | | | | |
| Z-score, mean | 1.6 | 1.0 | −0.6 | 1.6 | 0.9 | −0.7 |
| Femoral neck | n = 43 | | | n = 43 | | |
| Z-score, mean | 1.0 | 0.5 | −0.5 | 1.0 | 0.4 | −0.6 |
| Total hip | n = 43 | | | n = 43 | | |
| Z-score, mean | 1.0 | 0.6 | −0.4 | 1.0 | 0.5 | −0.5 |
| ⅓ radius | n = 41 | | | n = 41 | | |
| Z-score, mean | 0.3 | 0.3 | 0.0 | 0.3 | 0.3 | 0.0 |

*Groups A and B included all subjects who had both Week 26 and matching baseline, and Week 58 and match baseline scans, respectively, read by the central lab. Number of subjects in each group with an anatomical reading was the same, although the actual subjects were not identical in each group. [Two subjects in Group A were not in Group B, and two subjects in Group B were not in Group A].

Mean BMD Z-scores from baseline through 58-weeks of treatment with Compound 1 showed a trend towards normalization and stabilization of BMD.

Table 4 shows the DXA scan results of the lumbar spine L1-L4, femoral neck, total hip and distal 1/3 radius taken at a later time in the study, when more data points and also week 110 data was available.

TABLE 4
DXA scan results of up to week 110

| | Baseline | Week 26 | Week 58 | Week 110 |
|---|---|---|---|---|
| Region | (n = 57) | (n = 46) | (n = 46) | (n = 55) |
| Lumbar Spine | 1.6 | 1.0 | 0.9 | 0.7 |
| L1-L4 | | | | |
| Femoral Neck | 1.0 | 0.5 | 0.4 | 0.3 |
| Total Hip | 1.0 | 0.6 | 0.5 | 0.4 |
| Distal 1/3 | 0.4 | 0.3 | 0.3 | 0.2 |
| Radius* | | | | |

*n = 3 participants missing distal 1/3 radius corrected Z-scores at each time point

### BMD by Duration of Hypoparathyroidism

Patients were also segmented into subgroups based on the duration of their hypoparathyroidism: Hypoparathyroidism lasting more than 10 years; 5-10 years and less than 5 years. Data is shown in Table 5.

As expected, the duration of hypoparathyroidism correlated with more elevated Z-scores, with patients having hypoparathyroidism for more than 10 years having the highest BMD. This was particular pronounced at bone consisting predominantly of trabecular bone (lumbar spine, femoral neck and total hip), whereas cortical sites (distal 1/3 radius) were less affected. Continued treatment with compound 1 for 110 weeks was associated with larger numeric decreases in Z-scores in patients with longer duration of hypoparathyroidism and higher baseline Z-scores. The largest correction in Z-score occurs within the first 26 weeks, with stabilization of the effect out though 110 weeks. Mean Z-scores remained above 0 for the duration of treatment.

Treatment with compound 1 for 110 weeks did not lead to meaningful changes from baseline in Z-scores for cortical bone at the distal 1/3 radius.

TABLE 5
BMD by duration of hypoparathyroidism

Duration of Hypoparathyroidism

| | | >10 years | 5-10 years | | <5 years |
|---|---|---|---|---|---|
| | | (n = 26) | (n = 17) | | (n = 14) |
| Lumbar spine | | | | | |
| | Baseline | 2.209 (0.35) | 1.369 | (0.37) | 0.920 (0.36) |
| | Week 26 | 1.431 (0.44) | 0.840 | (0.37) | 0.500 (0.34) |
| | Week 58 | 1.373 (0.31) | 0.729 | (0.32) | 0.546 (0.33) |
| | Week 110 | 1.093 (0.31) | 0.454 | (0.32) | 0.428 (0.33) |
| | Femoral | | | | |
| | Neck | | | | |
| | Baseline | 1.397 (0.23) | 0.641 | (0.24) | 0.595 (0.26) |
| | Week 26 | 0.869 (0.32) | 0.260 | (0.25) | 0.367 (025) |
| | Week 58 | 0.797 (0.30) | 0.219 | (0.22) | 0.225 (0.22) |
| | Week 110 | 0.527 (0.22) | 0.047 | (0.20) | 0.333 (0.24) |
| | Total Hip | | | | |
| | Baseline | 1.354 (0.23) | 0.578 | (0.25) | 0.694 (0.29) |
| | Week 26 | 0.860 (0.30) | 0.411 | (0.28) | 0.358 (0.25) |
| | Week 58 | 0.840 (0.28) | 0.322 | (0.24) | 0.258 (0.23) |
| | Week 110 | 0.568 (0.22) | 0.074 | (0.23) | 0.316 (0.28) |
| | Distal 1/3 | | | | |
| | Baseline | 0.629 (0.24) | −0.138 | (0.35) | 0.452 (0.29) |
| | Week 26 | 0.513 (0.28) | 0.139 | (0.27) | 0.266 (0.22) |
| | Week 58 | 0.431 (0.29) | 0.125 | (0.26) | 0.298 (0.16) |
| | Week 110 | 0.371 (0.26) | −0.234 | (0.24) | 0.286 (0.21) |

In summary, treatment with compound 1 was well tolerated through week 110 and no safety signals have been identified.

### Dose Adjustments

Mean dose of Compound 1 was 18 ag PTH(1-34) (range: 12-21) for pooled subjects at Week 4, and the daily dose of Compound 1 remained steady at that level through the OLE. At Week 84, 50% (29/58) of the subjects did not require a dose adjustment from the last visit (Week 58). There were no subjects that decreased their dose of compound 1. Additionally, 31% (18/58) required only 1 dose increase of compound 1 and 19% (11/58) required more than 1 dose increase of compound 1. Mean doses of Compound 1 at various time points are shown in Table 6.

TABLE 6
Mean doses of Compound 1 at various time points

| | Mean Dose (μg PTH |
|---|---|
| Week | (1-34)/Day) (SD) |
| 4 | 17.9 (2.46) |
| 6 | 17.7 (3.02) |
| 8 | 17.8 (3.64) |
| 10 | 17.6 (4.22) |
| 12 | 17.7 (4.58) |
| 14 | 17.7 (4.84) |
| 18 | 17.9 (4.90) |
| 22 | 18.2 (4.88) |
| 26 | 18.3 (4.97) |
| 34 | 18.4 (5.09) |
| 42 | 18.7 (5.37) |
| 50 | 19.0 (5.55) |
| 58 | 19.6 (6.16) |
| 84 | 22.8 (9.47) |

During the OLE, for subjects taking active vitamin D at Visit 3, the dose of Compound 1 was titrated starting at Visit 4. For subjects not taking active vitamin D at Visit 3, the dose of Compound 1 was titrated starting at Visit 3. Starting at Visit 9, all subjects were titrated to optimal dose.

### EXAMPLE 2

Treatment of Hypoparathyroidism with Compound 1

Within two weeks before the first dose of Compound 1, serum 25(OH) vitamin D (also known as calcifediol, calcidiol, 25-hydroxycholecalciferol, 25-hydroxyvitamin D3) should be confirmed to be within the normal range.

At the time of initiation of treatment with Compound 1, serum calcium should be confirmed to be ≥7.8 mg/dL.

If the patient is taking active vitamin D at the time treatment with Compound 1 is initiated and:
if serum calcium is ≥8.3 mg/dL, active vitamin D (calcitriol) is discontinued on the same day as the first dose of Compound 1 is administered and the same dose of calcium supplements is maintained;
if serum calcium is <8.3 mg/dL, the dose of active vitamin D is reduced by ≥50% on the same day as the first dose of Compound 1 is administered and the same dose of calcium supplements is maintained.

If the patient is not taking active vitamin D at the time treatment with Compound 1 is initiated, calcium supplements are decreased by at least 1500 mg on the same day as the first dose of Compound 1 is administered. If calcium supplement doses of ≤1500 mg calcium/day are taken, calcium supplement doses are discontinued entirely.

If calcium supplements are indicated to meet dietary requirements, a continuation of dietary calcium supplements at doses of ≤600 mg/day may be considered instead of discontinuing entirely.

The recommended starting dose is 18 μg PTH(1-34) administered in the form of Compound 1 with dose adjustments in 3 μg PTH(1-34) increments thereafter. The dosage range of Compound 1 is 6 to 60 μg PTH(1-34)/day, which doses are provided in prefilled pens of 168 μg PTH(1-34)/0.56 mL (delivering doses of 6, 9 or 12 μg PTH(1-34)); 294 μg PTH(1-34)/0.98 mL (delivering doses of 15, 18, or 21 μg PTH(1-34)); and 420 μg PTH(1-34)/1.4 ml (delivering doses of 24, 27, or 30 μg PTH(1-34)). Each ml of Compound 1 formulation contains 3456 μg of Compound 1, corresponding to 300 μg of PTH(1-34) as active ingredient, and the following inactive ingredients: 1.18 mg succinic acid, 41.7 mg mannitol, 2.5 mg metacresol, 0.13 mg sodium hydroxide, and water for injection, with a pH of 3.7 to 4.3.

Compound 1 is inspected visually for particulate matter and discoloration prior to administration.

Compound 1 is administered subcutaneously daily to the abdomen or front of the thigh and the injection site is rotated daily. For doses of >30 μg PTH(1-34)/day two sequential injections are required, using different sites for each injection.

Serum calcium is measured within 7 to 14 days of the first dose of Compound 1 and appropriate adjustments in dosing of Compound 1, active vitamin D and calcium supplement are as follows:
if serum calcium is low (<8.3 mg/dL):
   if ≥7 days have passed since treatment with Compound 1 was started or the dose of Compound 1 was changed, the same calcium supplement and active vitamin D doses are continued, and the dose of Compound 1 by 3 μg is increased;
   if fewer than 7 days have passed since treatment with Compound 1 was started or the dose of Compound 1 was changed, calcium supplements and/or active vitamin D are increased toward prior doses based on physician's clinical judgement and the same dose of Compound 1 is continued;
if serum calcium is normal (8.3 to 10.6 mg/dL):
   if ≥7 days have passed since treatment with Compound 1 was started or since the dose of Compound 1 was changed and the patient is still taking active vitamin D, active vitamin D is discontinued, and the dose of Compound 1 is increased by 3 μg;
   if ≥7 days have passed since treatment with Compound 1 was started or since the dose of Compound 1 was changed and the patient is no longer taking active vitamin D, but is taking calcium supplements, wherein the calcium supplement is ≥1500 mg/day, the calcium supplement is decreased by ≥1500 mg and the dose of Compound 1 is increased by 3 μg;
   if ≥7 days have passed since treatment with Compound 1 was started or since the dose of Compound 1 was changed and the patient is no longer taking active vitamin D, but is taking calcium supplements, wherein the calcium supplement is less than 1500 mg/day, calcium supplements are discontinued, and the dose of Compound 1 is increased by 3 μg;
   if ≥7 days have passed since treatment with Compound 1 was started or since the dose of Compound 1 was changed and the patient is no longer taking active vitamin D and is no longer taking calcium supplements, the same dose of Compound 1 is continued;
   if fewer than 7 days have passed since treatment with Compound 1 was started or since the dose of Compound 1 was changed, the same dose of Compound 1, of the calcium supplement and active vitamin D is continued;
if serum calcium is high (10.7 to 11.9 mg/dL):
   if the patient is still taking active vitamin D, active vitamin D is discontinued and the same doses of Compound 1 and calcium supplement are continued;
   if the patient is not taking active vitamin D but takes calcium supplements at a dose of ≥1500 mg/day, calcium supplements are decreased by ≥1500 mg and the same dose of Compound 1 is continued;
   if the patient is not taking active vitamin D but takes calcium supplements, which calcium supplements are less than 1500 mg per day, calcium supplements are discontinued, and the same dose of Compound 1 is continued;
   if the patient is not taking active vitamin D and does not take calcium supplements, the dose of Compound 1 is decreased by 3 μg;
and if serum calcium is very high (≥12 mg/dL), withhold Compound 1 for 2 to 3 days, recheck serum calcium and:
   if subsequent serum calcium is <12 mg/dL, resume titration of Compound 1, active vitamin D and calcium supplements as described above using the most recent serum calcium value obtained; and
   if serum calcium remains ≥12 mg/dL, withhold Compound 1 for an additional 2 to 3 days, recheck serum calcium and proceed as described above.

Doses of >30 μg/day are administered as two single doses injected sequentially at different injection sites using two administrations as shown in Table 7.

TABLE 7
Scheme for doses of >30 μg/day

| Dose | Dosing Scheme |
|---|---|
| 33 μg PTH(1-34)/day | 15 μg PTH(1-34)/day + 18 μg PTH(1-34)/day |
| 36 μg PTH(1-34)/day | 18 μg PTH(1-34)/day + 18 μg PTH(1-34)/day |
| 39 μg PTH(1-34)/day | 18 μg PTH(1-34)/day + 21 μg PTH(1-34)/day |
| 42 μg PTH(1-34)/day | 21 μg PTH(1-34)/day + 21 μg PTH(1-34)/day |
| 45 μg PTH(1-34)/day | 21 μg PTH(1-34)/day + 24 μg PTH(1-34)/day |
| 48 μg PTH(1-34)/day | 24 μg PTH(1-34)/day + 24 μg PTH(1-34)/day |
| 51 μg PTH(1-34)/day | 24 μg PTH(1-34)/day + 27 μg PTH(1-34)/day |
| 54 μg PTH(1-34)/day | 27 μg PTH(1-34)/day + 27 μg PTH(1-34)/day |
| 57 μg PTH(1-34)/day | 27 μg PTH(1-34)/day + 30 μg PTH(1-34)/day |
| 60 μg PTH(1-34)/day | 30 μg PTH(1-34)/day + 30 μg PTH(1-34)/day |

Dose adjustments of Compound 1, active vitamin D and calcium supplements should be made on the same day. After any dose change in Compound 1, active vitamin D or calcium supplements, measure serum calcium within 7 to 14 days and monitor for clinical symptoms of hypocalcemia or hypercalcemia and adjust doses of Compound 1, active vitamin D and/or calcium supplements as described above.

The dose of Compound 1 may be increased as described above in increments of 3 μg if at least 7 days have elapsed since a prior dose change of Compound 1. Do not increase the dose of Compound 1 more often than every 7 days. The dose of Compound 1 may be reduced no more often than every 3 days in 3 μg increments in response to hypercalcemia as described above.

The maintenance dose should be the dose of Compound 1 that achieves serum calcium within the normal range, without the need for active vitamin D or therapeutic doses of calcium. Optionally, calcium supplementation sufficient to meet dietary requirements may be continued. Serum calcium may be measured per standard of care once a maintenance dose is achieved.

If a dose is missed by less than 12 hours, it may be taken as soon as possible. If a dose is missed by more than 12 hours, it may be skipped, and the next dose is then taken as scheduled.

Interruption of daily administration should be avoided to minimize serum PTH fluctuations. Interruption or discontinuation of Compound 1 may result in hypocalcemia. In patients interrupting or discontinuing Compound 1 for 3 or more consecutive doses, it is recommended to monitor for signs and symptoms of hypocalcemia and to consider measuring serum calcium. If indicated, treatment with calcium supplements and active vitamin D is resumed. It is recommended that administration of Compound 1 is resumed at the prescribed dose as soon as possible after an interruption. When resuming Compound 1 after an interruption, serum calcium should be measured and doses of Compound 1, active vitamin D and calcium supplements adjusted as described above.

1-36. (canceled)

37. A method of treating a patient having an increased bone mineral density (BMD), wherein the method comprises administering to said patient a pharmaceutically effective amount of a long-acting PTH compound.

38. The method of claim 37, wherein the reduction in BMD is a reduction in the Z-score of at least 0.1.

39. The method of claim 37, wherein the reduction in BMD is a reduction of BMD in trabecular bones.

40. The method of claim 37, wherein the reduction in BMD is measured in at least one region selected from the group consisting of lumbar spine L1 to L4, femoral neck and total hip.

41. The method of claim 37, wherein no reduction in BMD is observed in the distal 1/3 radius.

42. The method of claim 37, wherein the reduction in BMD does not result in a Z-score below 0.

43. The method of claim 37, wherein the reduction in BMD is associated with an initial increase in bone turnover markers that trends towards age- and sex-appropriate norms with prolonged use of the long-acting PTH compound.

44. The method of claim 37, wherein the patient having increased BMD is a patient having a disease selected from the group consisting of hypoparathyroidism; SAPHO syndrome; chronic infective osteomyelitis; osseous tuberous sclerosis; fluorosis; renal osteodystrophy; acromegaly; hepatitis C-associated osteosclerosis; myelofibrosis; mastocytosis; congenital conditions of reduced bone resorption such as osteopetrosis, pycnodysostosis, osteopoikilosis and melorheostosis; congenital conditions of increased bone formation such as sclerosteosis, van Buchem's disease, LRP5 HBM, LRP4 HBM, craniometaphyseal dysplasia; and conditions of disturbed formation and resorption such as Camurati Engelmann disease and Ghosal syndrome.

45. The method of claim 37, wherein the patient having increased BMD is a patient having hypoparathyroidism.

46. The method of claim 45, wherein the patient has had hypoparathyroidism for less than 5 years.

47. The method of claim 45, wherein the patient has had hypoparathyroidism for between 5 and 10 years.

48. The method of claim 45, wherein the patient has had hypoparathyroidism for more than 10 years.

49. The method of claim 37, wherein the patient having increased BMD is a patient having osteopetrosis.

50. The method of claim 37, wherein the long-acting PTH compound is administered to the patient in the form of a pharmaceutical composition comprising one or more long-acting PTH compound and at least one excipient.

51. The method of claim 37, wherein the long-acting PTH compound is administered to the patient once daily.

52. A method of treating hypoparathyroidism, wherein the method comprises the step of administering a long-acting PTH compound in a pharmaceutically effective amount in a dosage regimen, in which the dose of the long-acting PTH compound is increased in the course of the treatment and wherein such dosage regimen comprises the steps of
(i) titrating the dose of the long-acting PTH compound administered to the patient to result in normal serum calcium levels in the patient and maintaining the patient on such dose for a first time period;
(ii) increasing the dose of the long-acting PTH compound for a second time period directly following the first time period by a factor of at least 1.1; and
(iii) optionally increasing the dose of the long-acting PTH compound for a third or further subsequent time period by a factor of at least 1.1.

53. The method of claim 52, wherein the first time period is at least 5 months.

54. The method of claim 52, wherein the second time period is at least 1 month.

55. The method of claim 37, wherein the long-acting PTH compound is of formula (Ia) or (Ib) or a pharmaceutically acceptable salt thereof 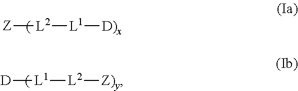
wherein
each -D is individually a PTH moiety;
each -L¹- is individually a linker moiety covalently and reversibly connected to -D;
each -L²- is individually a single chemical bond or a spacer moiety;
each —Z is individually a carrier moiety, such as a fatty acid derivative or a polymer;
x is an integer selected from the group consisting of 1, 2, 3, 4, 5, 6, 7, 8, 9, 10, 11, 12, 13, 14, 15, 16, 17, 18, 19, 20, 21, 22, 23, 24 and 25; and
y is an integer selected from the group consisting of 2, 3, 4 and 5.

56. The method of claim 55, wherein the long-acting PTH compound is a compound of formula (Ia).

57. The method of claim 55, wherein the long-acting PTH compound is a compound of formula (Ia) with x=1.

58. The method of claim 37, wherein the long-acting PTH compound is of formula (IIf-i): 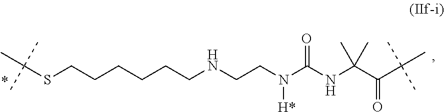
wherein
the unmarked dashed line indicates the attachment to a nitrogen of -D through an amide bond; and
the dashed line marked with the asterisk indicates attachment to a moiety 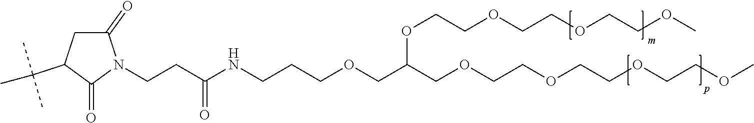
wherein
m and p are independently an integer ranging from and including 400 to 500.

59. The method of claim 58, wherein the unmarked dashed line indicates attachment to the N-terminal amine of a PTH moiety of SEQ ID NO:51.

60. The method of claim 37, wherein the long-acting PTH compound is administered to the patient in the form of a pharmaceutical composition comprising one or more long-acting PTH compound and at least one excipient.

61. The method of claim 37, wherein the long-acting PTH compound is administered to the patient once daily.

62. The method of claim 37, wherein the long-acting PTH compound is administered to the patient once a week.

63. A pharmaceutical composition comprising a compound of formula (IIf-ii) 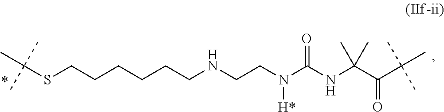
wherein
the unmarked dashed line indicates the attachment to the N-terminal amine of a PTH moiety of SEQ ID NO:51; and
the dashed line marked with the asterisk indicates attachment to a moiety 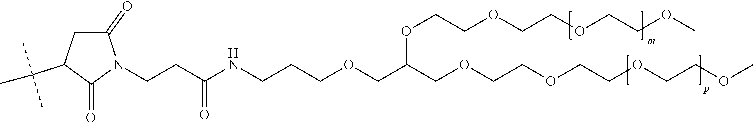
wherein
m and p are independently an integer ranging from and including 400 to 500,
wherein 1 ml of the pharmaceutical composition comprises 3456 μg of the long-acting PTH compound of formula (IIf-ii), 1.18 mg succinic acid, 41.7 mg mannitol, 2.5 mg metacresol, 0.13 mg sodium hydroxide and water for injection.

64. The pharmaceutical composition of claim 63, wherein the pharmaceutical composition has a pH of 3.7 to 4.3.

65. A method of treating a patient having hypoparathyroidism, wherein the method comprises administering to said patient a pharmaceutically effective amount of a compound of formula (IIf-ii) 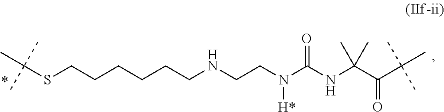
wherein
the unmarked dashed line indicates the attachment to the N-terminal amine of a PTH moiety of SEQ ID NO:51; and
the dashed line marked with the asterisk indicates attachment to a moiety 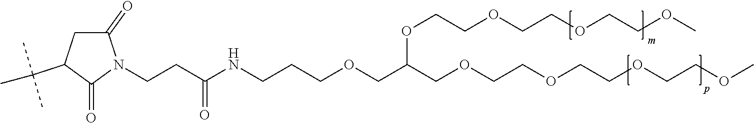
wherein
m and p are independently an integer ranging from and including 400 to 500,
or a pharmaceutical composition comprising the compound of formula (IIf-ii), wherein the treatment is initiated by
a) confirming that the serum 25(OH) vitamin D of the patient is within the normal range within two weeks before the first dose of the compound of formula (IIf-ii) is administered to the patient and serum calcium is ≥7.8 mg/dL at the initiation of treatment;
b) if the patient is taking active vitamin D at the time treatment with the compound of formula (IIf-ii) is initiated:
b-i) maintaining the same dose of calcium supplements and discontinue active vitamin D on the same day as the first dose of the compound of formula (IIf-ii) is administered, if serum calcium is ≥8.3 mg/dL; or
b-ii) reducing the dose of active vitamin D by ≥50% on the same day as the first dose of the compound of formula (IIf-ii) is administered and maintaining the same dose of calcium supplements, if serum calcium is <8.3 mg/dL; or
if the patient is not taking active vitamin D at the time treatment with the compound of formula (IIf-ii) is initiated:
b-iii) decreasing calcium supplements by at least 1500 mg on the same day as the first dose of the compound of formula (IIf-ii) is administered; and
c) optionally continuing dietary calcium supplements at doses of ≤600 mg/day, if calcium supplements are indicated to meet dietary requirements.

66. The method of claim 65, wherein the treatment further comprises the steps of
d) administering a starting dose of 18 μg PTH(1-34)/day in the form of the compound of formula (IIf-ii), followed by daily administrations of the same dose;
e) measuring serum calcium within 7 to 14 days of the first administration of the compound of formula (IIf-ii); and
f) adjusting the dose of the compound of formula (IIf-ii), active vitamin D and/or calcium supplement.

67. The method of claim 66, wherein the dose adjusting of step f) are performed as follows:
f-i) if serum calcium is <8.3 mg/dL:
if ≥7 days have passed since treatment with the compound of formula (IIf-ii) was started or the dose of the compound of formula (IIf-ii) was changed, the same calcium supplement and active vitamin D doses are continued, and the dose of the compound of formula (IIf-ii) is increased by 3 μg; or
if fewer than 7 days have passed since treatment with the compound of formula (IIf-ii) has started or the dose of the compound of formula (IIf-ii) was changed, calcium supplements and/or active vitamin D are increased toward prior doses based on physician's clinical judgement and the same dose of the compound of formula (IIf-ii) is continued;
f-ii) if serum calcium ranges from 8.3 to 10.6 mg/dL:
if ≥7 days have passed since treatment with the compound of formula (IIf-ii) was started or since the dose of the compound of formula (IIf-ii) was changed and the patient is still taking active vitamin D, active vitamin D is discontinued, and the dose of the compound of formula (IIf-ii) is increased by 3 μg;
if ≥7 days have passed since treatment with the compound of formula (IIf-ii) was started or since the dose of the compound of formula (IIf-ii) was changed and the patient is no longer taking active vitamin D, but is taking calcium supplements, wherein the calcium supplement is ≥1500 mg/day, the calcium supplement is decreased by ≥1500 mg and the dose of the compound of formula (IIf-ii) is increased by 3 μg;
if ≥7 days have passed since treatment with the compound of formula (IIf-ii) was started or since the dose of the compound of formula (IIf-ii) was changed and the patient is no longer taking active vitamin D, but is taking calcium supplements, wherein the calcium supplement is less than 1500 mg/day, calcium supplements are discontinued, and the dose of the compound of formula (IIf-ii) is increased by 3 μg;
if ≥7 days have passed since treatment with the compound of formula (IIf-ii) was started or since the dose of the compound of formula (IIf-ii) was changed and the patient is no longer taking active vitamin D and is no longer taking calcium supplements, the same dose of the compound of formula (IIf-ii) is continued; or
if fewer than 7 days have passed since treatment with the compound of formula (IIf-ii) was started or since the dose of the compound of formula (IIf-ii) was changed, the same dose of the compound of formula (IIf-ii), of the calcium supplement and active vitamin D is continued;
f-iii) if serum calcium ranges from 10.7 to 11.9 mg/dL:
if the patient is still taking active vitamin D, active vitamin D is discontinued and the same doses of the compound of formula (IIf-ii) and calcium supplement are continued;
if the patient is not taking active vitamin D but takes calcium supplements at a dose of 1500 mg/day, calcium supplements are decreased by 1500 mg and the same dose of the compound of formula (IIf-ii) is continued;
if the patient is not taking active vitamin D but takes calcium supplements, which calcium supplements are less than 1500 mg per day, calcium supplements are discontinued, and the same dose of the compound of formula (IIf-ii) is continued; or
if the patient is not taking active vitamin D and does not take calcium supplements, the dose of the compound of formula (IIf-ii) is decreased by 3 μg; or
f-iv) if serum calcium is ≥12 mg/dL:
withhold the compound of formula (IIf-ii) for 2 to 3 days, recheck serum calcium and
if subsequent serum calcium is <12 mg/dL, resume titration of the compound of formula (IIf-ii), active vitamin D and calcium supplements as described in steps f-i) to f-iii) using the most recent serum calcium value obtained; and
if serum calcium remains ≥12 mg/dL, withhold the compound of formula (IIf-ii) for an additional 2 to 3 days, recheck serum calcium and proceed as described in the previous step.

68. The method of claim 66, wherein the treatment further comprises the steps of
g) administration of a daily maintenance dose; and
h) measuring serum calcium per standard of care.
